SEQUENCE LISTING (56 sequences)

SEQ ID NO: 1 - DNA, 5723 bp, Homo sapiens
cggcgaacagacgttctttctcctccatgcagttacacaaaaggagggctacggaaactaaaagtttcggggcctctggctcggtgtgtggagaaaagagaaaacctggagacgggatatgaagatcaatgatgcagactgatggttttgatgaagctgggcatttataactagattcattaaggaatacaaagaaaatacttaaagggatcaataatggtgtcttctggttgcagaatgcgaagtctgtggtttatcattgtaatcagcttcttaccaaatacagaaggtttcagcagagcagctttaccatttgggctggtgaggcgagaattatcctgtgaaggttattctatagatctgcgatgcccgggcagtgatgtcatcatgattgagagcgctaactatggtcggacggatgacaagatttgtgatgctgacccatttcagatggagaatacagactgctacctccccgatgccttcaaaattatgactcaaaggtgcaacaatcgaacacagtgtatagtagttactgggtcagatgtgtttcctgatccatgtcctggaacatacaaataccttgaagtccaatatgaatgtgtcccttacatttttgtgtgtcctgggaccttgaaagcaattgtggactcaccatgtatatatgaagctgaacaaaaggcgggtgcttggtgcaaggaccctcttcaggctgcagataaaatttatttcatgccctggactccctatcgtaccgatactttaatagaatatgcttctttagaagatttccaaaatagtcgccaaacaacaacatataaacttccaaatcgagtagatggtactggatttgtggtgtatgatggtgctgtcttctttaacaaagaaagaacgaggaatattgtgaaatttgacttgaggactagaattaagagtggcgaggccataattaactatgccaactaccatgatacctcaccatacagatggggaggaaagactgatatcgacctagcagttgatgaaaatggtttatgggtcatttacgccactgaacagaacaatggaatgatagttattagccagctgaatccatacactcttcgatttgaagcaacgtgggagactgtatacgacaaacgtgccgcatcaaatgcttttatgatatgcggagtcctctatgtggttaggtcagtttatcaagacaatgaaagtgaaacaggcaagaactcaattgattacatttataatacccgattaaaccgaggagaatatgtagatgttcccttccccaaccagtatcagtatattgctgcagtggattacaatccaagagataaccaactttacgtgtggaacaataacttcattttacgatattctctggagtttggtccacctgatcctgcccaagtgcctaccacagctgtgacaataacttcttcagctgagctgttcaaaaccataatatcaaccacaagcactacttcacagaaaggccccatgagcacaactgtagctggatcacaggaaggaagcaaagggacaaaaccacctccagcagtttctacaaccaaaattccacctataacaaatatttttcccctgccagagagattctgtgaagcattagactccaaggggataaagtggcctcagacacaaaggggaatgatggttgaacgaccatgccctaagggaacaagaggaactgcctcatatctctgcatgatttccactggaacatggaaccctaagggccccgatcttagcaactgtacctcacactgggtgaatcagctggctcagaagatcagaagcggagaaaatgctgctagtcttgccaatgaactggctaaacataccaaagggccagtgtttgctggggatgtaagttcttcagtgagattgatggagcagttggtggacatccttgatgcacagctgcaggaactgaaacctagtgaaaaagattcagctggacggagttataacaaggcaattgttgacacagtggacaaccttctgagacctgaagctttggaatcatggaaacatatgaattcttctgaacaagcacatactgcaacaatgttactcgatacattggaagaaggagcttttgtcctagctgacaatcttttagaaccaacaagggtctcaatgcccacagaaaatattgtcctggaagttgccgtactcagtacagaaggacagatccaagactttaaatttcctctgggcatcaaaggagcaggcagctcaatccaactgtccgcaaataccgtcaaacagaacagcaggaatgggcttgcaaagttggtgttcatcatttaccggagcctgggacagttccttagtacagaaaatgcaaccattaaactgggtgctgattttattggtcgtaatagcaccattgcagtgaactctcacgtcatttcagtttcaatcaataaagagtccagccgagtatacctgactgatcctgtgctttttaccctgccacacattgatcctgacaattatttcaatgcaaactgctccttctggaactactcagagagaactatgatgggatattggtctacccagggctgcaagctggttgacactaataaaactcgaacaacgtgtgcatgcagccacctaaccaattttgcaattctcatggcccacagggaaattgcatataaagatggcgttcatgaattacttcttacagtcatcacctgggtgggaattgtcatttcccttgtttgcctggctatctgcatcttcaccttctgctttttccgtggcctacagagtgaccgaaatactattcacaagaacctttgtatcaaccttttcattgctgaatttattttcctaataggcattgataagacaaaatatgcgattgcatgcccaatatttgcaggacttctacactttttctttttggcagcttttgcttggatgtgcctagaaggtgtgcagctctacctaatgttagttgaagtttttgaaagtgaatattcaaggaaaaaatattactatgttgctggttacttgtttcctgccacagtggttggagtttcagctgctattgactataagagctatggaacagaaaaagcttgctggcttcatgttgataactactttatatggagcttcattggacctgttaccttcattattctgctaaatattatcttcttggtgatcacattgtgcaaaatggtgaagcattcaaacactttgaaaccagattctagcaggttggaaaacattaagtcttgggtgcttggcgctttcgctcttctgtgtcttcttggcctcacctggtcctttgggttgctttttattaatgaggagactattgtgatggcatatctcttcactatatttaatgctttccagggagtgttcattttcatctttcactgtgctctccaaaagaaagtacgaaaagaatatggcaagtgcttcagacactcatactgctgtggaggcctcccaactgagagtccccacagttcagtgaaggcatcaaccaccagaaccagtgctcgctattcctctggcacacagagtcgtataagaagaatgtggaatgatactgtgagaaaacaatcagaatcttcttttatctcaggtgacatcaatagcacttcaacacttaatcaaggacattcactgaacaatgccagggatacaagtgccatggatactctaccgctaaatggtaattttaacaacagctactcgctgcacaagggtgactataatgacagcgtgcaagttgtggactgtggactaagtctgaatgatactgcttttgagaaaatgatcatttcagaattagtgcacaacaacttacggggcagcagcaagactcacaacctcgagctcacgctaccagtcaaacctgtgattggaggtagcagcagtgaagatgatgctattgtggcagatgcttcatctttaatgcacagcgacaacccagggctggagctccatcacaaagaactcgaggcaccacttattcctcagcggactcactcccttctgtaccaaccccagaagaaagtgaagtccgagggaactgacagctatgtctcccaactgacagcagaggctgaagatcacctacagtcccccaacagagactctctttatacaagcatgcccaatcttagagactctccctatccggagagcagccctgacatggaagaagacctctctccctccaggaggagtgagaatgaggacatttactataaaagcatgccaaatcttggagctggccatcagcttcagatgtgctaccagatcagcaggggcaatagtgatggttatataatccccattaacaaagaagggtgtattccagaaggagatgttagagaaggacaaatgcagctggttacaagtctttaatcatacagctaaggaattccaagggccacatgcgagtattaataaataaagacaccattggcctgacgcagctccctcaaactctgcttgaagagatgactcttgacctgtggttctctggtgtaaaaaagatgactgaaccttgcagttctgtgaatttttataaaacatacaaaaactttgtatatacacagagtatactaaagtgaattatttgttacaaagaaaagagatgccagccaggtattttaagattctgctgctgtttagagaaattgtgaaacaagcaaaacaaaactttccagccattttactgcagcagtctgtgaactaaatttgtaaatatggctgcaccatttttgtaggcctgcattgtattatatacaagacgtaggctttaaaatcctgtgggacaaatttactgtaccttactattcctgacaagacttggaaaagcaggagagatattctgcatcagtttgcagttcactgcaaatcttttacattaaggcaaagattgaaaacatgcttaaccactagcaatcaagccacaggccttatttcatatgtttcctcaactgtacaatgaactattctcatgaaaaatggctaaagaaattatattttgttctattgctagggtaaaataaatacatttgtgtccaactgaaatataattgtcattaaaataattttaaagagtgaagaaaatattgtgaaaagctcttggttgcacatgttatgaaatgttttttcttacactttgtcatggtaagttctactcattttcacttcttttccactgtatacagtgttctgctttgacaaagttagtctttattacttacatttaaatttcttattgccaaaagaacgtgttttatggggagaaacaaactctttgaagccagttatgtcatgccttgcacaaaagtgatgaaatctagaaaagattgtgtgtcacccctgtttattcttgaacagagggcaaagagggcactgggcacttctcacaaactttctagtgaacaaaaggtgcctattcttttttaaaaaaataaaataaaacataaatattactcttccatattccttctgcctatatttagtaattaatttattttatgataaagttctaatgaaatgtaaattgtttcagcaaaattctgcttttttttcatccctttgtgtaaacctgttaataatgagcccatcactaatatccagtgtaaagtttaacacggtttgacagtaaataaatgtgaattttttcaagtaaaaaaaaaaaaaaaaaa

SEQ ID NO: 2 - DNA, 26 bp, Artificial Sequence
  Description of Artificial Sequence Synthetic oligonucleotide
tctgcatttcctgtaattttgcatgc

SEQ ID NO: 3 - DNA, 1345 bp, Homo sapiens
ggaacaggatgcccctgtcccgctggttgagatctgtgggggtcttcctgctgccagccccctactgggcaccccgggagaggtggctgggttccctacggcggccctccctggtgcacgggtacccagtcctggcctggcacagtgcccgctgctggtgccaagcgtggacagaggaacctcgagccctttgctcctccctcagaatgaacggagaccagaattcagatgtttatgcccaagaaaagcaggatttcgttcagcacttctcccagatcgttagggtgctgactgaggatgagatggggcacccagagataggagatgctattgcccggctcaaggaggtcctggagtacaatgccattggaggcaagtataaccggggtttgacggtggtagtagcattccgggagctggtggagccaaggaaacaggatgctgatagtctccagcgggcctggactgtgggctggtgtgtggaactgctgcaagctttcttcctggtggcagatgacatcatggattcatcccttacccgccggggacagatctgctggtatcagaagccgggcgtgggtttggatgccatcaatgatgctaacctcctggaagcatgtatctaccgcctgctgaagctctattgccgggagcagccctattacctgaacctgatcgagctcttcctgcagagttcctatcagactgagattgggcagaccctggacctcctcacagccccccagggcaatgtggatcttgtcagattcactgaaaagaggtacaaatctattgtcaagtacaagacagctttctactccttctaccttcctatagctgcagccatgtacatggcaggaattgatggcgagaaggagcacgccaatgccaagaagatcctgctggagatgggggagttctttcagattcaggatgattaccttgacctctttggggaccccagtgtgaccggcaaaattggcactgacatccaggacaacaaatgcagctggctggtggttcagtgtctgcaacgggccactccagaacagtaccagatcctgaaggaaaattacgggcagaaggaggctgagaaagtggcccgggtgaaggcgctatatgaggagctggatctgccagcagtgttcttgcaatatgaggaagacagttacagccacattatggctctcattgaacagtacgcagcacccctgcccccagccgtctttctggggcttgcgcgcaaaatctacaagcggagaaagtgacctagagattgcaagggcggggagaggaggctctcaataaataatcgtgtaaccttaaaaaaaaaaaaaaaaaaaaa

SEQ ID NO: 4 - DNA, 4178 bp, Homo sapiens
agcttttagccgcccgtctttgtaaggagaccactgagacgagcgggagcgcggagcagcagcctctgctgccctgactttttaagaaatctcaatgaactatttgtagagaatcactgatccggcctgcaagcattttgcacggcaaaaatatcgatcagtgttaagtgaagatcacattttatatgcgatcttgacttttttgtcttacattatatttttatagattttgttataaacatggtgctgggaaaggtgaagagtttgacaataagctttgactgtcttaatgacagcaatgtccctgtgtattctagtggggataccgtctcaggaagggtaaatttagaagttactggggaaatcagagtaaaatctcttaaaattcatgcaagaggacatgcgaaagtacgctggactgaatctagaaacgccggctccaatactgcctatacacagaattacactgaagaagtagagtatttcaaccataaagacatcttaattgggcacgaaagagatgatgataattccgaagaaggcttccacactattcattcaggaaggcatgaatatgcattcagcttcgagcttccacagacaccactcgctacctcattcgaaggccgacatggcagtgtgcgctattgggtgaaagccgaattgcacaggccttggctactaccagtaaaattaaagaaggaatttacagtctttgagcatatagatatcaacactccttcattactgtcaccccaagcaggcacaaaagaaaagacactctgttgctggttctgtacctcaggcccaatatccttaagtgccaaaattgaaaggaagggctataccccaggtgaatcaattcagatatttgctgagattgagaactgctcttcccgaatggtggtgccaaaggcagccatttaccaaacacaggccttctatgccaaagggaaaatgaaggaagtaaaacagcttgtggctaacttgcgtggggaatccttatcatctggaaagacagagacgtggaatggcaagttgctgaaaattccaccagtttctccctctatcctcgactgtagtataatccgcgtggaatattcactaatggtatatgtggatattcctggagctatggatttatttcttaatttgccacttgtcatcggtaccattcctctacatccatttggtagcagaacctcaagtgtaagcagtcagtgtagcatgaatatgaactggctcagtttatcacttcctgaaagacctgaagcaccacccagctatgcagaagtggtaacagaggaacaaaggcggaacaatcttgcaccagtgagtgcttgtgatgactttgagagagcccttcaaggaccactgtttgcatatatccaggagtttcgattcttgcctccacctctttattcagagattgatccaaatcctgatcagtcagcagatgatagaccatcctgcccctctcgttgaaggaacacttggttgaatcaagttgatgtgggttccgaactgtatctcttccggctgaggacagagaagtatcttggagacacgtttcagaggaagtggaattacttttgcccagaaaaatggcgaatacatgaaacaaccagtgatcatgctttagaagcctacagcaacattctgagactgctccaacatgcttgaagatctaagcttttctcttttaaaactggcacatactcagagcagtcttcttagcctatggtcgtacgtgtcaagacatcacgttgtaaagagggatgatttccttcttttgatttgaaaatttgcacatgctcaatgcttacattgtgcggttcgacgtcactacagcttctttttttttttttttttttttctatttttgccagactcttgatactcttaaaacttgtttgtggtcagcacaacaaggaacaaaacaaagctttgaaaaaactttaacatgaaaaaacgcactgacatttttttttatttaatatagcctggactttacctgcgtatgcacatgctcagaattgtctactaggctgactatgtatcacctcttcagcttggatccaattgtggatttatttacaaacatcaaatgccttcaagccaatcctttttgctgtatgttttgcagcctactgtagtagatacgcaacagataatgtgggaaaaaaagagataagaggaggaagctaataagagactgtcaagattgtataccttcttggtttcttttaagaatttgttgcctttctactattacagcaaagcagcattttgttactgactgcctaaaatcacttaatctcaggtgaacgcatcacttgccaaactgttggaatgctatttgtgttttgttgcactgtttttttcgtttgtttgtttgtttatttggttggctttttggagagggaaatttggaaacgggacatacacaaaagttacacacccacattccctttttatcatgacatacaagaagaaactagcagagctaagaatggagtgaagaaaggcagtatggcaggcaccagcaaagagttgagggctgttgctcttaaaaattattttttttattattattttgaaagtatggaagttttccattcactggggaaaggagggaaaagtgcatttatttttatacagagttacttaattacctccaaaacacatatgttggaaatcgcttttgctggtgcaaagtatattaatgagcaggaatacatacattgaggttatgaatagagagctcaatttgtacctttgctgtcttgctcaagcttggtatggcatgaaaactcgactttattccaaaagtaacttcaaaatttaaaatactagaacgtttgctgcgataaatcttttggatttttgtgtttttctaatgagaatactgtttttcattacctaaagaacaatttgctaaacatgagaaatcactcactttgattatgtatagattacataggaagaacaatcacatcagtaagttatagtttatattaaaggtaattttctgttggctcataacaaatataccagcattcatgatagcatttcagcattttccaaggtaccaagtgtacttattttgttgttgttgttgttgttgtattttagaaggaattcagctctgatgtttttaaagaaaaccagcatctctgatgttgcaacatacgtgtaaaatgggtgttacatctatcctgccatttaaccccacagttaataaagtggctgaaaataatagtagctctggcttggtgcttgacctggttaaatactgtcttaaagctcatacaaaacaaataggcttttccataagtggcctttaagaaaacatggaagacaattcatgtttgacaaatgctgacagggtgaagaaagcccagtgtaaaaatgaatcgcgttttaagtgattcggttaaagagtttgggctcccgtagcaaactaatactagataataaggaaatgggggtgaaatatttttttattgttgaatcattttgtgaatgtccccctcaaaaaaagctaatggaatatttggcataaagggcatttggtggttttatttttgtttgagggggattgtcagaaaatcccttttctctcttacgtctaactgactagggaacaattgttgatatgcatagcattggaatacttgtcattatatactcttacaaataacacatgaagcaagaatgaccaatattctgataattggcactggatcacaaaatgtgataaaactttaaatgtataaaactttatcaaataaagttttattttcccctttaaaatgtatttctttagaggcattacttttttaaaaatattggtcaattcctgacataagatgtgaggttcacagttgtattccagtattcaagatagattcctgatttttcaattaggaaaagtaaaatccaaaatgttagcaaaacaaagtgcaatattaaatgtttgctttatagattatattctatggctgtttgtaatttctctttttttccttttttatttggtgctgaatatgtccttgtaggctctgttttaagaaaacaatatgtgggaaatgatttaatttttcctattgctcttccttgtggaaaataaagtgttttgtttttttctgttttgtataaaaaaaaaaaaaaaaaaa

SEQ ID NO: 5 - DNA, 343 bp, Homo sapiens
atcaaaagcttgagcacaaacagatacctcagcccggagtttctgttcctgttcttgaagagacagactggtgagcatgcaaaattacaggaaatgcagagaacaaaatgggcagagcaaccaaaactgtggtgtttgcatcaaatacaggtcaagagtaaacttattttcctatgaaattcaagaacatgttgaaactggaaagagcgggacaggctggtagcaccttttaaagaccaagagaggccgcctcattaaatattaagaacttggaggaaagaggtggatttacactgataaaaggttcatttaaaattccatgaggtcaataaattaccactta

SEQ ID NO: 6 - DNA, 29 bp, Artificial Sequence
  Description of Artificial Sequence Synthetic oligonucleotide
ctgctgcaggatccaaatcctgcagcagt

SEQ ID NO: 7 - DNA, 6 bp, Artificial Sequence
  Description of Artificial Sequence Synthetic oligonucleotide
ctgcag

SEQ ID NO: 8 - DNA, 6 bp, Artificial Sequence
  Description of Artificial Sequence Synthetic oligonucleotide
cagcag

SEQ ID NO: 9 - DNA, 18 bp, Artificial Sequence
  Description of Artificial Sequence Synthetic oligonucleotide
ggatcgataagtcaaaaa

SEQ ID NO: 10 - DNA, 40 bp, Artificial Sequence
  Description of Artificial Sequence Synthetic oligonucleotide
nntttttgactcatcgatgggaccggcagcaaaatgttcg

SEQ ID NO: 11 - DNA, 6 bp, Artificial Sequence
  Description of Artificial Sequence Synthetic oligonucleotide
atcgat

SEQ ID NO: 12 - DNA, 5 bp, Artificial Sequence
  Description of Artificial Sequence Synthetic oligonucleotide
gagtc

SEQ ID NO: 13 - DNA, 5 bp, Artificial Sequence
  Description of Artificial Sequence Synthetic oligonucleotide
ttttt

SEQ ID NO: 14 - DNA, 18 bp, Artificial Sequence
  Description of Artificial Sequence Synthetic oligonucleotide
gatccccgggcacacaat

SEQ ID NO: 15 - DNA, 16 bp, Artificial Sequence
  Description of Artificial Sequence Synthetic oligonucleotide
cgattgtgtgcccggg

SEQ ID NO: 16 - DNA, 22 bp, Artificial Sequence
  Description of Artificial Sequence Synthetic primer
caggaagatggctgtgagggac

SEQ ID NO: 17 - DNA, 20 bp, Artificial Sequence
  Description of Artificial Sequence Synthetic primer
cggatctcgacggtatcggt

SEQ ID NO: 18 - DNA, 20 bp, Artificial Sequence
  Description of Artificial Sequence Synthetic primer
ctttggatttgggaatctta

SEQ ID NO: 19 - DNA, 22 bp, Artificial Sequence
  Description of Artificial Sequence Synthetic primer
gcaacagacatacaaactaaag

SEQ ID NO: 20 - DNA, 25 bp, Artificial Sequence
  Description of Artificial Sequence Synthetic oligonucleotide
agcggagaaagtgacctagagattg

SEQ ID NO: 21 - DNA, 25 bp, Artificial Sequence
  Description of Artificial Sequence Synthetic oligonucleotide
caatctctaggtcactttctccgct

SEQ ID NO: 22 - DNA, 21 bp, Artificial Sequence
  Description of Artificial Sequence Synthetic oligonucleotide
ccauguacauggcaggaautt

SEQ ID NO: 23 - DNA, 21 bp, Artificial Sequence
  Description of Artificial Sequence Synthetic oligonucleotide
auuccugccauguacauggtt

SEQ ID NO: 24 - DNA, 26 bp, Artificial Sequence
  Description of Artificial Sequence Synthetic oligonucleotide
ccacagacaccactcgctacctcatt

SEQ ID NO: 25 - RNA, 19 nt, Artificial Sequence
  Description of Artificial Sequence Synthetic oligonucleotide
gaacugcucuucccgaaug

SEQ ID NO: 26 - DNA, 26 bp, Artificial Sequence
  Description of Artificial Sequence Synthetic oligonucleotide
tctgcatttcctgtaattttgcatgc

SEQ ID NO: 27 - RNA, 21 nt, Artificial Sequence
  Description of Artificial Sequence Synthetic oligonucleotide
uaauauuuaaugaggcggccu

SEQ ID NO: 28 - DNA, 18 bp, Artificial Sequence
  Description of Artificial Sequence Synthetic primer
aaggccaaccgcgagaag

SEQ ID NO: 29 - DNA, 18 bp, Artificial Sequence
  Description of Artificial Sequence Synthetic primer
ggcgtacagggatagcac

SEQ ID NO: 30 - DNA, 4923 bp, Homo sapiens
gcgcgcccgccgcccgggcggaggagaggagcgcgcggccgcgcagagcaagctgagccgagccgagccgagctgggggcgcagagcgcgggaggcggcggcggcgcggagcccaggtggctccgctgccggatgggagtgccccagtgtgctggatgaagctggcgcatgcaccatgtcatcatgtgtctctagccagcccagcagcaaccgggccgccccccaggatgagctggggggcaggggcagcagcagcagcgaaagccagaagccctgtgaggccctgcggggcctctcatccttgagcatccacctgggcatggagtccttcattgtggtcaccgagtgtgagccgggctgtgctgtggacctcggcttggcgcgggaccggcccctggaggccgatggccaagaggtcccccttgacacctccgggtcccaggcccggccccacctctccggtcgcaagctgtctctgcaagagcggtcccagggtgggctggcagccggtggcagcctggacatgaacggacgctgcatctgcccgtccctgccctactcacccgtcagctccccgcagtcctcgcctcggctgccccggcggccgacagtggagtctcaccacgtctccatcacgggtatgcaggactgtgtgcagctgaatcagtataccctgaaggatgaaattggaaagggctcctatggtgtcgtcaagttggcctacaatgaaaatgacaatacctactatgcaatgaaggtgctgtccaaaaagaagctgatccggcaggccggctttccacgtcgccctccaccccgaggcacccggccagctcctggaggctgcatccagcccaggggccccattgagcaggtgtaccaggaaattgccatcctcaagaagctggaccaccccaatgtggtgaagctggtggaggtcctggatgaccccaatgaggaccatctgtacatggtgttcgaactggtcaaccaagggcccgtgatggaagtgcccaccctcaaaccactctctgaagaccaggcccgtttctacttccaggatctgatcaaaggcatcgagtacttacactaccagaagatcatccaccgtgacatcaaaccttccaacctcctggtcggagaagatgggcacatcaagatcgctgactttggtgtgagcaatgaattcaagggcagtgacgcgctcctctccaacaccgtgggcacgcccgccttcatggcacccgagtcgctctctgagacccgcaagatcttctctgggaaggccttggatgtttgggccatgggtgtgacactatactgctttgtctttggccagtgcccattcatggacgagcggatcatgtgtttacacagtaagatcaagagtcaggccctggaatttccagaccagcccgacatagctgaggacttgaaggacctgatcacccgtatgctggacaagaaccccgagtcgaggatcgtggtgccggaaatcaagctgcacccctgggtcacgaggcatggggcggagccgttgccgtcggaggatgagaactgcacgctggtcgaagtgactgaagaggaggtcgagaactcagtcaaacacattcccagcttggcaaccgtgatcctggtgaagaccatgatacgtaaacgctcctttgggaacccattcgagggcagccggcgggaggaacgctcactgtcagcgcctggaaacttgctcacgaagcaaggcagcgaagacaacctccagggcaccgacccgccccccgtgggggaggaggaagtgctcttgtgagaggcagtccctgcgtggaaagttgctgggcccccgcccccggctcccccgcacgcatgcatccactgcggccggaggaggccatggagcccgagtagctgcctggatcgctcgacctcgcatgcgcgccgcgtcgcctctggggggctgctgcaccgcgtttccatagcagcatgtcctacggaaacccagcacgtgtgtagagcctcgatcgtcatctctggttatttgttttttcctttgttgttttaaaggggacaaaaaaaaaaaaaggacttgactccatgacgtcgaccgtggccgctggctggctggacaggcgggtgtgaggagttgcagacccaaacccacgtgcattttgggacaattgctttttaaaacgtttttatgccaaaaatccttcattgtgattttcagaaccacgtcagatataccaagtgactgtgtgtggggtttgacaactgtggaaaggcgagcagaaaactccggcggtctgaggccatggaggtggttgctgcatttgagagggagtagggggctagatgtggctcctagtgcaaaccggaaaccatggcaccttccagagccgtggtctcaaggagtcagagcagggctggccctcagtagctgcagggagctttgatgcaacttatttgtaagaaggatttttaaattttttatgggtagaattgtagtcaggaaaacagaaagggcttgaaatttaataagtgctgctggaaggggattttccaagcctggaagggtattcagcagctgtggtggggaaacatttctcctgaaagactgaacgtgtttcttcatgacagctgctcaaagcaggtttctgagatagctgaccgagctctggtaaatctctttgtcaaattacgaaaacttcagggtgaaatcctatgcttccatgtacattacatggcttaagattaaacaaaaacatttttcaagtctctaactagagtgaactctagagcacagtagttcagaaactatttagagcttccaggatatatttcacagcttcaggcatgtgatcagttagagccgatgaaacctatgcccgcctgtatatatattagcagcttagctagttcataacctgtatattctaaagactgctaaggttttgttttcattttaaatcctagctgattgttgtggtcaatgaaatacccagtttctggagggccaggtgggaaatgctttcactggaccaacacacaaatgatcatcctgaggatctgagcttccctagactccacacaataaccttggggcacccttttagagaagactgttgaaacccacagcactcgttggggtatgaggaaaccagggcttggcacaggaagttcccctttgtagctaaaagtccagaaagaaagggttcatctttttgacttccaactgatattgggaagtttggttgaggttcaagtgtgactccttccagagccacaggtaggggagtgtgaagttgagggggaggaaagctggaaggactctgccttgggagattcccagctctgctttccagcgcttggtggaatctgggctggggaaagacggcaccgggaaactctgcttccccattgtttccatctgatcagctgtggtgtgaggacttctcagacaaaggcaaggcctcgtgcccctgcccagcccattcatggagccctgggccttcttggcttccatagatcctaagctcttgactgtagtttagccagacttgttttgctatcttataagcagttcagaattagggaatgctggttttgaagagcaaaggacaggtagtctagagagggtcgtctggcctgcttgctgggtctttgtaacccagcacttcctcttgccctcctggctttatgtttatggggagaggactcaatagctccaccccttctggcaccagatggggcttggttagtttgcaataagcaccttgcagaggttaaagccagcgggtccctagtcttaggcccagcctgcttgtgtgggctctggcctggcctggtggctggcccagggggcagcagtgcttagagcttctgcagggcttctcttgtttacacagctgcatcagacaatgccatttctccccaccacggaaccttccatctaagatttcttccagggaatgccagcaatcaggcagcacccagctgtgggggcagtggggtgggggagacccacattgatgactttttttttttcttttaatgaagaaacaccaaagaaagctgtggaaaggacctgccccacatgaaaaggataagccaagatggctgtaaacacagagcatttgagctgccactcttggagcacattgatttttcaaaagccagctctgtcaggaaaggaggtgctgttatgagcagctcttccagtgggcaaagaggacgcccataatttcttccattgctagctcatctgtgggaccaatttggtgtaagcaacctgtggcctgcacttgtggcctcgaaggaagcacaaaccctccatccacttcccatttcctctgcccttttccacctcccccttccatcccaccagctgccagtggctcccagaaagccttattgagccccttgttgacacttggggctgcggaggcctctccctactggtctggcctttcctgagaggcaggtcttccgtcctcagagcctttctggaacaaggagaatgcctgtgcaggtggacacacaggcctggcctgtcgctctcacttgtcttccagcggggagcttcacgttgccgagtggaagaaccatgacctccacttgcttccaaggtgctagggaagtttcagggtacgctggttcccctctccagctggaggccgagtttctggggactgcagatttttctactctgtgatcgattcaatgcccgatgcttctgtttcattcccgaccctttctactatgcattttccttttatcaggtgtataaagttaaatactgtgtatttatcactaaaaagtacatgaacttaagagacaactaagcctttcgtgtttttccacaggtgtttaagcttctctgtacagttgaaataaacagacagcaaaatggtgccaaaaaaaaaaaaaaaaaaa

SEQ ID NO: 31 - protein, 5 aa, Artificial Sequence
  Description of Artificial Sequence Synthetic peptide
GGGGC

SEQ ID NO: 32 - DNA, 21 bp, Artificial Sequence
  Description of Artificial Sequence Synthetic oligonucleotide
gcuccuauggugucgucaatt

SEQ ID NO: 33 - DNA, 21 bp, Artificial Sequence
  Description of Artificial Sequence Synthetic oligonucleotide
uugacgacaccauaggagctt

SEQ ID NO: 34 - DNA, 23 bp, Artificial Sequence
  Description of Artificial Sequence Synthetic oligonucleotide
uaauauuuaaugaggcggccutt

SEQ ID NO: 35 - DNA, 23 bp, Artificial Sequence
  Description of Artificial Sequence Synthetic oligonucleotide
aggccgccucauuaaauauuatt

SEQ ID NO: 36 - DNA, 2173 bp, Homo sapiens
gatgaataactccacagctcctcctggaccctgcgcgggagcaggcggctcctgtgctgtaaagaaaattgattcgcttgcggctcacctcagtcgaggaagccctggaatgttcagcagaacaccaccactgtgacatggggctgtggcagtgggagacaccgcatgtggcgtgggtgtgtgtggcggcgtggctcgcactcagtcctgtgtaggagaggaaagggaatcaaaagcttgagcacaaacagatacctcagcccggtaagctgcagctctgctcaactgcgtcttctctcagccctccacacacgctcacccccactcccacacacacacacacacacacacacacacaccatctcccgagggctgacctcctctggcctcagcctgcagggtgtgggcagagaagggcatctgggacgtggtgccagtgaggagcccagttggctggcactgggcccatttgaaggtgtctcagacatttggccagtatgtctttctcaggggtttggtcacaaaggatggactcttcccacccagaggatgcagggaaagcacactgtgtctttccggtcattggatcctctccctttccccaggcagctcgcctggccacaccgttggagtgaaccctcactgccctcaaggacaacagcagggtgtcacccagagccggatgagggatgccagcaggtgcccccacgagtggggccttggcccaagcagagcttcccctgaaggtgccatcagccagggcagctctgtcccctcctgctctccatcttatatgtattctaaccaggaaaaatgtgatagcacatgggtagcctaggcagtgaataaatacctcagatgtcctcctgcaaaaaaaaaaaaaaaaaaaaaaataggaggctgaaacctagaactgagaaaaatctgagtttttattaaaaaaaagcacgtttttactttctgatatccacctcagcttttgttctttaaaatgggatcaatgtcattacacaattttcattaaaatcatgtaaaaagcaccacgctgtgcaaaagatgggcccaaatactctgcaaagatcattgcacgtaaatcagatcctttccctctacctgtaggagtttctgttcctgttcttgaagagacagactggtgagcatgcaaaattacaggaaatgcagagaacaaaatgggcagagcaaccaaaactgtggtgtttgcatcaaatacaggtcaagagtaaacttattttcctatgaaattcaagaacatgttgaaactggaaagagcgggacaggctggtagcaccttttaaagaccaagagaggccgcctcattaaatattaagaacttggaggaaagaggtggatttacactgataaaaggttcatttaaaattccatgaggtcaataaattaccacttaagatgccatttcccaaaatgtgtcctgaagaatgcttgttttaaatgagggtggaggggtagagggaaaaaatcctgtggtccaattgatttatgcactgtatctcagatggagtttgacaagaaatgttggccaggcgcggtgtatcaaacctgtaatcccagcactttgggaggctgaggtgggaaggatcacttgagaccaggagtttgagaccagcctggatgacataaggagaccccatctctacaaataattaaaaaattagccaggtgtgtcagtgcacacctgtggtctcagctactcaggaggctgaggcaagaggatcacctaaggccaggaggttgaggctgcagtgagatgtgatggcaccactgcactccagcctgggtgactgagtgagacccagtctcaaaagaacaaaacaaaacaaaacaaaacaaaacaaaacaaaacaaaaaaccagacaataaattgttgagttaccaaaggccctgttaacgcctgcaacaaaaaagattgctgaatcttgcttagtccagtcttttccaaacttgtttgaccttcaacccctttcctcttcctctgcacattcatctacataacaaatgtacagagggcctcctccaggccagtcactgttctaggtcctggggattcagcagtgaagaaaacaaaactctcccttggcagactgtgtaattaggaggagacagaaaataaaccaatagataaatcaaaccagaa

SEQ ID NO: 37 - protein, 4 aa, Artificial Sequence
  Description of Artificial Sequence Synthetic peptide
AASA

SEQ ID NO: 38 - protein, 4 aa, Artificial Sequence
  Description of Artificial Sequence Synthetic peptide
AALA

SEQ ID NO: 39 - DNA, 33 bp, Artificial Sequence
  Description of Artificial Sequence Synthetic oligonucleotide
aannnnnnnnnnnnnnnnnnnnnnnnnnnnntt

SEQ ID NO: 40 - DNA, 17 bp, Artificial Sequence
  Description of Artificial Sequence Synthetic oligonucleotide
acattttgctgccggtc

SEQ ID NO: 41 - DNA, 5 bp, Artificial Sequence
  Description of Artificial Sequence Synthetic oligonucleotide
gactc

SEQ ID NO: 42 - DNA, 25 bp, Artificial Sequence
  Description of Artificial Sequence Synthetic primer
tgaccggcaaaattggcactgacat

SEQ ID NO: 43 - DNA, 23 bp, Artificial Sequence
  Description of Artificial Sequence Synthetic primer
ccttcacccgggccactttctca

SEQ ID NO: 44 - DNA, 85 bp, Artificial Sequence
  Description of Artificial Sequence Synthetic oligonucleotide
aagtcaaaaatacagttagaggacagatagtagtaactgctgcagcagttactactatctgtcctctaactgtaggggatccgtg

SEQ ID NO: 45 - DNA, 86 bp, Artificial Sequence
  Description of Artificial Sequence Synthetic oligonucleotide
aagtcaaaaannnnnnnnnnnnnnnnnnnnnnnnnnactgctgcagcagtnnnnnnnnnnnnnnnnnnnnnnnnnggggatccgtg

SEQ ID NO: 46 - DNA, 84 bp, Artificial Sequence
  Description of Artificial Sequence Synthetic oligonucleotide
aagtcaaaaatacagttagagacagatagtagtaactgctgcagcagttactactatctgtcctctaactgtaggggatccgtg

SEQ ID NO: 47 - DNA, 85 bp, Artificial Sequence; identical to SEQ ID NO: 44
  Description of Artificial Sequence Synthetic oligonucleotide

SEQ ID NO: 48 - protein, 4 aa, Unknown
  Description of Unknown ARRDC3 peptide motif
PPSY

SEQ ID NO: 49 - protein, 4 aa, Unknown
  Description of Unknown ARRDC3 peptide motif
PPLY

SEQ ID NO: 50 - protein, 54 aa, Homo sapiens
EAPPSYAEVVTEEQRRNNLAPVSACDDFERALQGPLFAYIQEFRFLPPPLYSEI

SEQ ID NO: 51 - protein, 54 aa, Bos sp.
EAPPSYAEVVTEEQRRNNLAPQSACDDFERALQGPLFAYIQEFRFLPPPLYSEI

SEQ ID NO: 52 - protein, 54 aa, Rattus sp.; identical to SEQ ID NO: 50

SEQ ID NO: 53 - protein, 54 aa, Mus sp.
EAPPSYAEVVTEEQRRNNLAPVQACDDFERALQGPLFAYIQEFRFLPPPLYSEI

SEQ ID NO: 54 - protein, 53 aa, Unknown
  Description of Unknown Frog polypeptide
EAPPSYAEVVTEEQRQNLMSSNACDNFELALQGPLFAYIQEFRFLPPPLYSEV

SEQ ID NO: 55 - protein, 50 aa, Danio rerio
EAPPAYAEVVTEEQRQNCLEVSPGRENYDGPLFAYIQEFRFRPPPPYSEI

SEQ ID NO: 56 - DNA, 10 bp, Artificial Sequence
  Description of Artificial Sequence Synthetic primer
aaaaaaaaaa

(19) United States
(12) Patent Application Publication
(10) Pub. No.: US 2013/0090371 A1
(43) Pub. Date: Apr. 11, 2013

(54) METHODS AND COMPOSITIONS FOR INHIBITION OF BETA2-ADRENERGIC RECEPTOR DEGRADATION

(71) Applicant: Quan Lu, Newton, MA (US)

(72) Inventor: Quan Lu, Newton, MA (US)

(73) Assignee: PRESIDENT AND FELLOWS OF HARVARD COLLEGE, Cambridge, MA (US)

(21) Appl. No.: 13/642,144

(22) PCT Filed: Apr. 20, 2011
(86) PCT No.: PCT/US11/33275

§ 371 Date: Dec. 20, 2012

Related U.S. Application Data
(60) Provisional application No. 61/326,089, filed on Apr. 20, 2010.

(51) Int. Cl. A61K 31/663; A61K 45/06; A61K 31/675; A61K 31/7088; A61K 31/724

(52) U.S. Cl. CPC A61K 31/663; A61K 31/7088; A61K 31/724; A61K 31/675; A61K 45/06
USPC 514/44 A; 514/102; 514/103; 514/108; 514/58; 514/80; 514/89; 514/94; 514/107

(57) ABSTRACT

The present invention generally relates to compositions and kits comprising a β2-AR agonist and a modulator of a β2-AR regulator gene, where the modulator of the β2-AR regulator gene inhibits the internalization and/or degradation of the β2-ad-renergic receptor (β2-AR). More specifically, the present invention relates to the use of an agonist of β2-adrenergic receptor (β2-AR) and an agent which inhibits agonist induced β2-adrenergic receptor (β2-AR) internalization and/or degradation in method for the treatment of a respiratory disorder in a subject.

### CROSS REFERENCE TO RELATED APPLICATIONS

This application claims benefit under 35 U.S.C. 119(e) of U.S. Provisional Patent Application Ser. No. 61/326,089 filed on Apr. 20, 2010, the contents of each are incorporated herein in their entity by reference.

### FIELD OF THE INVENTION

The present invention generally relates to the use of a combination of a β2-adrenergic receptor (β2-AR) agonist and an agent which inhibits the internalization and/or degradation of the β2-adrenergic receptor (β2-AR). More specifically, the present invention relates to the use of an agonist of β2-adrenergic receptor (β2-AR) and an agent which inhibits agonist induced β2-adrenergic receptor (β2-AR) internalization and/or degradation for the treatment of respiratory disorders in a subject.

### BACKGROUND OF THE INVENTION

Asthma is an increasingly prevalent chronic lung disease that affects about 300 million people worldwide, many of whom are children¹. There is currently no cure for asthma but a variety of therapies have been developed and used to alleviate the symptoms of the disease². A group of drugs, known as β-agonists, function as bronchodilators to reduce airway smooth muscle constriction and airway narrowing, hallmark symptoms of asthma^{3,4}. β-agonists exert their effects by targeting the β2-adrenergic receptor (β2-AR) in the lung^{5,6}. Binding of the agonists to the receptor activates the receptor-associated stimulatory G protein, leading to a signaling cascade that ultimately results in airway smooth muscle relaxation and bronchodilation⁷.

Adrenergic receptors (AR) are members of the G-protein coupled receptors that are composed of a family of three receptor sub-types: α1 (_{A, B, D}), α2 (_{A, B, C}), and β(_{1, 2, 3}). These receptors are expressed in tissues of various systems and organs of mammals and the proportions of the α and the β receptors are tissue dependant. For example, tissues of bronchial smooth muscle express largely β2-AR while those of cutaneous blood vessels contain exclusively α-AR subtypes.

β2-AR agonists have become a mainstay of asthma therapy, but suffer from two important limitations. First, long-term use of β-agonists significantly reduces the efficacy of the therapy. Second, there is significant variability in response to this therapy among patients. Both limitations can be attributed largely to a process known as receptor down-regulation, in which continuous stimulation of the receptor by the agonist leads to reduced receptor level at the cell surface. Such reduction in functional β2-AR level slows the responsiveness of target lung tissue cells to agonists, thus severely limiting the efficacy of agonists in asthma therapy⁸⁻¹⁰. As a result, repetitive or continuous use of β2-AR agonists causes loss of bronchodilation and/or protection against bronchoconstriction⁸. Many adverse effects, such as loss of asthma control and longer durations of asthma exacerbation may also be attributed to receptor down-regulation caused by prolonged agonist use¹¹. The clinical importance of receptor down-regulation is also supported by studies on β2-AR polymorphisms¹²⁻¹⁴. β2-AR alleles that display accelerated degradation in vitro are associated with altered sensitivity to β2-adrenergic bronchodilators in asthmatic patients¹⁵⁻¹⁷.

Some β-agonists with long lasting bronchodilation effect (long acting β-agonists) are traditionally used for maintenance therapy of moderate and severe asthma and COPD. While improving the patients' respiratory functions efficiently, use of β-agonists is limited because the often cause both pulmonary and extrapulmonary adverse effects. Drug tolerance is also major respiratory adverse effect in regular use of β-agonists, which leads to reduced bronchodilator response and poorer disease control.

Both receptor desensitization and receptor down regulation play important roles in β-agonists tolerance by reducing the amount of cell surface receptor. Receptor desensitization happens immediately upon exposure to β-agonists. Such rapid desensitization processes include β-arrestin mediated uncoupling of G-protein complexes from receptors and internalization of receptors through endocytosis. Internalized β2AR may recycle back to cell surface or enter lysosomes for degradation. Receptor down regulation happens after prolonged exposure to β-agonists, a condition in which most receptors undergo degradation and very few remain on cell surfaces. Although these receptor regulations are important for maintaining tissue homeostasis, they contribute to the loss of drug potency or effectiveness. In many cases, asthma and COPD treatment requires regular or long-term use of β-agonists, and that results in loss of bronchodilation and/or protection against bronchoconstriction.

Despite its critical role in determining the long-term efficacy of and patient response to β-agonist therapy, the β-agonist-induced down-regulation remains incompletely defined. It has been established that the β2-AR sub-type is involved in respiratory diseases such as such as asthma, chronic bronchitis, nervous system injury, and premature labor. Currently, a number of drugs e.g., albuterol, formoterol, isoprenolol, or salmeterol having β-AR agonist activities are being used to treat asthma. However, these drugs have limited utility as they are either non-selective thereby causing adverse side effects such as muscle tremor, tachycardia, palpitations, and restlesness, or have short duration of action and/or slow onset time of action. Thus such use of β2-AR agonists that target the beta-2 adrenergic receptors in the lung have significant practical limitations, such as (i) long-term use of beta-agonists leads to receptor downregulation that significantly reduces the efficacy of the therapy, and (ii) there are significant disparities in individual patient responses to the therapy due to differences in beta-2 receptor downregulation. While some studies have increased the understanding of β2AR receptor desensitization and down regulation, there is yet no efficient therapeutic target identified within the receptor regulation process or to prevent receptor degradation and thus prevent reduced β-agonist efficacy. Accordingly, there is a need for methods to improve the duration of β2-AR agonists that have increased potency and/or longer duration of action.

### SUMMARY OF THE INVENTION

The present invention generally relates to methods and compositions to prevent beta 2-adrenergic receptor (β2-AR) degradation. In particular, the present invention provides compositions for inhibition of regulators of beta 2-adrenergic receptor degradation, for example, modulators of β2-AR regulator genes which include FDPS (farnesyl diphosphate synthase), ARRDC3 (arrestin domain containing 3) latrophilin 2, CaMKK2, AAEST and KIAA0786 genes. In some embodiments, modulators of the β2-AR regulator genes can be any agent, e.g., RNAi inhibitors, and in some embodiments, can be an inhibitor of any one or a combination of FDPS, ARRDC3, CaMKK2 or AAEST β2-AR regulator gene, or an activator of latrophilin 2. In some embodiments, an inhibitor of FDPS is a small molecule inhibitor, such as a bisphosphate compound (e.g. alendronate) and cholesterol depleting agent, methyl-beta-cyclodextrin (MbCD).

Another invention relates to methods for the treatment of a respiratory disease or disorder in a subject, comprising administering a modulator of a β2-AR regulator gene, e.g. at least one or more of, or any combination of: an inhibitor of FDPS (farnesyl diphosphate synthase), an inhibitor of ARRDC3 (arrestin domain containing 3), an inhibitor of CaMKK2, or an inhibitor of AAEST, and/or an activator of latrophilin 2, and a β2-AR agonist.

In particular in some embodiments, a β2-AR agonist activates the β2-AR. In some embodiments, the administration of a modulator of a β2-AR regulator gene, e.g. an inhibitor of FDPS, or inhibitor of ARRDC3, an inhibitor of CaMKK2 or an inhibitor of AAEST, or an activator of latrophilin 2, and a β-agonist to a subject is substantially simultaneously, sequentially or separately. Thus, the combination of β2-AR agonists with a modulator of a β2-AR regulator gene is useful in the treatment and prevention of respiratory diseases, such as asthma, chronic obstructive pulmonary disease (COPD), and chronic bronchitis. In some embodiments, the combination of a β2-AR agonist with a modulator of a β2-AR regulator gene is useful in the treatment and prevention of the nervous system injury and premature labor.

Another aspect of the present invention provides a method for increasing β2-AR agonist function, e.g. β2 adrenergic receptor agonists function in a subject, comprising administering simultaneously, sequentially or separately to a subject a composition comprising a β2-AR agonist and a composition comprising an agent which modulates a β2 adrenergic receptor regulator gene, e.g. an inhibitor (e.g. antagonist) of FDPS and/or ARRDC3 and/or CaMKK2, and/or AAEST and/or an activator (i.e. agonist) of latrophilin 2.

Another aspect of the present invention relates to a composition comprising, in admixture, at least one β2-AR agonist and at least one agent which modulates a β2-AR regulator gene, e.g. an inhibitor (e.g. antagonist) of FDPS and/or ARRDC3 and/or CaMKK2 and/or AAEST, and/or an activator (i.e. agonist) of latrophilin 2.

In some embodiments, the admixture as described herein is administered to a subject, for example for the treatment of a respiratory disorder in a subject. In another embodiment, the admixture composition is in a form suitable for administration by inhalation.

Another aspect of the present invention relates to a kit comprising a preparation of a β2-AR agonist, e.g. a β2-AR agonist, at least one agent which modulates a β2 AR gene, e.g. an inhibitor (e.g. antagonist) of FDPS and/or ARRDC3 and/or CaMKK2 and/or AAEST, and/or an activator (i.e. agonist) of latrophilin 2 and optionally, instructions for the simultaneous, sequential or separate administration of the preparation to a subject in need thereof.

One aspect of the present invention relates to a method for treating a respiratory disease in a subject, comprising administering simultaneously, sequentially or separately, a composition comprising a β2 adrenergic receptor agonist and a composition comprising a modulator of at least one β2 adrenergic receptor regulator gene.

Another aspect of the present invention relates to a method for increasing β2 adrenergic receptor agonist function in a subject, comprising administering simultaneously, sequentially or separately, a composition comprising a β2 adrenergic receptor agonist and a composition a subject a modulator of a β2 adrenergic receptor regulator gene. In such embodiments, the modulator of a β2 adrenergic receptor regulator gene is any one or a combination of an inhibitor (e.g. antagonist) of FDPS and/or ARRDC3 and/or CaMKK2, and/or AAEST and/or an activator (i.e. agonist) of latrophilin 2.

Another aspect of the present invention provides a composition comprising, in admixture, a β2 adrenergic receptor agonist and a modulator of a β2 adrenergic receptor regulator gene. In some embodiments, the composition further comprises a pharmaceutical acceptable carrier. In such embodiments, the modulator of a β2 adrenergic receptor regulator gene is any one or a combination of an inhibitor (e.g. antagonist) of FDPS and/or ARRDC3 and/or CaMKK2 and/or AAEST, and/or an activator (i.e. agonist) of latrophilin 2.

In some embodiments, a modulator is an inhibitor and the β2 adrenergic receptor regulator gene is farnesyl diphosphate synthase (FDPS). In some embodiments, the modulator is an inhibitor and the β2 adrenergic receptor regulator gene is arrestin domain containing 3 (ARRDC3). In some embodiments, the modulator is an inhibitor and the β2 adrenergic receptor regulator gene is CaMKK2. In some embodiments, the modulator is an inhibitor and the β2 adrenergic receptor regulator gene is AAEST. In some embodiments, the modulator is a nucleic acid modulator, such as a RNAi agent, such as a gene silencing RNAi agent.

In some embodiments, a modulator of a β2 adrenergic receptor regulator gene which is an inhibitor of the farnesyl diphosphate synthase (FDPS) β2-AR regulator gene is a gene silencing RNAi having a sequence of SEQ ID NO: 20, or a small molecule inhibitor, such as a bisphosphate compound, e.g. aldendonate. In some embodiments, the small molecule inhibitor is methyl-beta-cyclodextrin (MbCD). In some embodiments, a modulator which is a gene silencing RNAi agent which downregulates or decreases FDPS mRNA levels is a 25-nt hairpin sequence (5′-AGC GGA GAA AGT GAC CTA GAG ATT G-3′ (SEQ ID NO: 20) as disclosed herein in the Examples. In some embodiments, a modulator of FDPS β2-AR regulator gene is a gene silencing RNAi, such as, for example, a shRNA sequence which is: CAATCTCTAGGTCACTTTCTCCGCT (SEQ ID NO: 21). In some embodiments, a modulator which is a gene silencing RNAi of the FDPS β2-AR regulator gene is 5′-CCA UGU ACA UGG CAG GAA U(dT)(dT)-3′ (sense strand) (SEQ ID NO: 22), and 5′-AUU CCU GCC AUG UAC AUG G(dT)(dT)-3′ (antisense strand) (SEQ ID NO: 23) which is commercially available, for example from Sigma-Aldrich or from Dharmacon.

In some embodiments, a modulator of a β2 adrenergic receptor regulator gene which is an inhibitor of the ARRDC3 β2-AR regulator gene is a gene silencing RNAi, such as, for example, a shRNA sequence which is CCACAGACACCACTCGCTACCTCATT (SEQ ID NO: 24). In some embodiments, a modulator for use in the methods, compositions and kits disclosed herein is a gene silencing RNAi of the ARRDC3 β2-AR regulator gene which is a siRNA having the sequence: GAACUGCUCUUCCCGAAUG (SEQ ID NO: 25).

In some embodiments, a modulator of a β2 adrenergic receptor regulator gene which is an inhibitor of the CaMKK2 β-AR regulator gene is a gene silencing RNAi, such as, for example, a shRNA sequence which is a CaMKK2-specific siRNA 5′ GCUCCUAUGGUGUCGUCAAdTdT (CaMKK2 siRNA #1) (SEQ ID NO: 32) and 5′ UUGACGACACCAUAGGAGCdTdT (CaMKK2 siRNA #2) (SEQ ID NO: 33). In some embodiments, a modulator for use in the methods, compositions and kits disclosed herein is a gene silencing RNAi of the CaMKK2 β2-AR regulator gene which is a siRNA having the sequence: 5′ GCUCCUAUGGUGUCGUCAAdTdT (SEQ ID NO: 32) and 5′ UUGACGACACCAUAGGAGCdTdT (SEQ ID NO: 33). Other siRNA targeting CaMKK2 inhibition are commercially available and encompassed for use in the compositions and methods as disclosed herein.

In some embodiments, a modulator of a β2 adrenergic receptor regulator gene which is an inhibitor of the AAEST β-AR regulator gene is a gene silencing RNAi, such as, for example, a siRNA sequence which is a AAEST-specific siRNA 5′ UAAUAUUUAAUGAGGCGGCCUdTdT (SEQ ID NO: 34) and/or 5′ AGGCCGCCUCAUUAAAUAUUAdTdT (SEQ ID NO: 35). In some embodiments, a modulator for use in the methods, compositions and kits disclosed herein is a gene silencing RNAi of the AAEST β2-AR regulator gene which is a siRNA having the sequence: 5′ UAAUAUUUAAUGAGGCGGCCUdTdT (SEQ ID NO: 34) and/or 5′ AGGCCGCCUCAUUAAAUAUUAdTdT (SEQ ID NO: 35). Other siRNA targeting AAEST inhibtion can be readily produced by one of ordinary skill in the art and are encompassed for use in the compositions and methods as disclosed herein.

In some embodiments, a modulator of a β2 adrenergic receptor regulator gene is an activator, for example, where the β2 adrenergic receptor regulator gene to be activated is latrophilin 2. In some embodiments, the modulator is a nucleic acid modulator, such as a RNAi agent, such as a gene activating RNAi agent. In some embodiments, a modulator which is an activator of the latrophilin 2 β2-AR regulator gene is SEQ ID NO: 2 or targets a nucleic acid sequence or region in the EST AA496068 (SEQ ID NO: 5). In some embodiments, a modulator of latrophilin 2 β2-AR regulator gene is a gene activating RNAi, such as, for example, the shRNA sequence for kiaa0786 (latrophilin 2, AA496068), which is TCTGCATTTCCTGTAATTTTGCATGC (SEQ ID NO: 26). In some embodiments, a modulator which is a gene activating RNAi of AA496068 (e.g. a 5′ upstream region of latrophilin 2) for activation of the latrophilin 2 β2-AR regulator gene is a siRNA having the sequence: UAAUAUUUAAUGAGGCGGCCU (SEQ ID NO: 27).

In some embodiments, a respiratory disease is chronic obstructive pulmonary disease (COPD), or asthma. In some embodiments, subject is a human. In some embodiments, a subject is identified to have a respiratory disease prior to administration of the composition comprising a β2 adrenergic receptor agonist and a composition comprising a modulator of at least one β2 adrenergic receptor regulator gene. In some embodiments, the method further comprises an initial step of selecting a subject with a respiratory disease prior to the simultaneous, sequential or separate administration of a composition comprising a β2 adrenergic receptor agonist and a composition comprising a modulator of at least one β2 adrenergic receptor regulator gene. In some embodiments, the method further comprises a first step of selecting a subject in need of β2-AR agonist therapy prior to the simultaneous, sequential or separate administration of a composition comprising a β2 adrenergic receptor agonist and a composition comprising a modulator of at least one β2 adrenergic receptor regulator gene.

In some embodiments, the composition as disclosed herein is useful for the treatment of a respiratory disorder in a subject, for example a human subject. In some embodiments, the composition as disclosed herein is in a form suitable for administration by inhalation. In some embodiments, the composition as disclosed herein is useful for the treatment of a respiratory disorder in a subject, where the subject has been selected to be in need of β2-AR agonist therapy, and/or has a respiratory disorder, for example by a physician or clinician of ordinary skill in the art.

Another aspect of the invention relates to a kit comprising a preparation of a beta-2 adrenergic receptor agonist, a modulator of a β2 adrenergic receptor regulator gene and optionally, instructions for the simultaneous, sequential or separate administration of the preparation to a subject in need thereof. In some embodiments, the kit comprises at least one modulators for the β2 adrenergic receptor regulator gene is selected from the group of farnesyl diphosphate synthase (FDPS); arrestin domain containing 3 (ARRDC3); CaMKK2, AAEST or latrophilin 2.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIGS. 1A-1F show the Enzymatic conversion of cDNA into shRNA expressing library. FIG. 1A is a schematic outline of the enzymatic procedure. FIG. 1B shows gel photos of the intermediate products (indicated by arrowheads). M1, 100-bp ladder; M2, 10-bp ladder; Lane 1, cDNA fragment; Lane 2, DNse I digestion; Lane 3, U adaptor ligation and EcoP15I digestion; Lane 4, Priming adaptor ligation and primer extension; Lane 5, MlyI+ClaI digestion. FIG. 1C shows a schematic of cloning into a vector, e.g., a lentiviral vector. FIG. 1D shows a schematic of the sequencing of the hairpin sequence. FIG. 1E shows the pooling of ESTs collections containing ˜28,000 genes and amplification by PCR and converted to a shRNA library. FIG. 1F shows the products of the EST library after DNase I digest, where the DNase I digested EST fragments are used for adding U-adapters comprising a EcoP15I restriction recognition site.

FIGS. 2A-2B show the knockdown of target gene expression by shRNA expressing library prepare from cDNA encoding SMURF2 or ARRDC1. FIG. 2A show an immunoblot analysis in 293T cells. In upper panel, cells were co-transfected with 800 ng DNA of individual shRNA expressing clones for SMURF2, or empty vector pLentiSuper2 (first lane), together with 200 ng DNA of target gene Flag-SMURF2 and 50 ng DNA of non-target control Flag-UEV3. Expression of both target and control genes were detected by using Flag-specific antibody. In lower panel, cells were co-transfected with 800 ng DNA of individual shRNA expressing clones for ARRDC1, or empty vector pLentiSuper2 (first lane), together with 200 ng DNA of target gene ARRDC1-eGFP and 50 ng DNA of non-target control eGFP. Expression of both target and control genes were detected by using GFP-specific antibody. FIG. 2B shows a histogram of the inhibition efficiency of 60 shRNA clones for ARRDC1. The expression level of ARRDC1-eGFP was normalized to that of eGFP. Inhibition efficiency was showed as percentage related to the normalized expression level of the empty vector.

FIGS. 3A-3C show FACS enrichment of FITC-positive population from the shRNA expressing library transduced 293β2AR* cells. FIG. 3A shows a flow diagram of the assay using the shRNA library to screen for targets genes which modulate agonist induced β2-AR internalization. FIG. 3B shows a schematic of β2-AR agonist induced degradation of β2-AR in the presence of shRNA from the shRNA library used in the screen. Cells were treated with 10 μM isoproterenol (+Iso) for 16 h or untreated (−Iso), and stained with FITC-conjugated Flag-specific antibody (+Ab) or unstained (−Ab). FITC-positive population was collected from the isoproterenol treated cells and expanded for repeated FACS enrichments. Total of 4 enrichments were performed. 293β2AR* cells which have been transduced with a shRNA which targets a β2-AR regulator gene will have less degradation defects and a more normal β2-AR distribution on the cell surface. FIG. 3C shows flow cytometry results from FACs sorting from a first, second, third and forth enrichment. In the fourth enrichment, FACs positive cells are 29% of the cells, whereas only 1% of the cells were FACs positive from the first enrichment step.

FIGS. 4A-4D show FDPS regulates the amount of Cell Surface β2AR in 293βAR* cells. FIG. 4A shows inhibition of β2AR degradation by FDPS-specific shRNA. Cells were transduced with Lentivirus expressing FDPS-specific shRNA or empty vector, and treated with 10 μM isoproterenol for 3 h. The expression of Flag-β2AR or β-Actin was detected by immunoblot using Flag-specific or β-Actin-specific antibodies respectively. FIG. 4B shows the knockdown efficiency of FDPS-specific shRNA and siRNA. Cells were transduced with Lentivirus expressing FDPS-specific shRNA or empty vector, or transfected with FDPS-specific siRNA or non-targeting Scrambled (SCR) siRNA. The RNA levels were determined by real time RT-PCR and normalized to that of β-Actin. Error bars represented the standard deviations of replicates. Statistic analysis was using student's t-test. FIG. 4C shows inhibition of β2AR degradation by FDPS-specific siRNA. Cells were transfected with FDPS-specific siRNA or SCR siRNA, and treated with 10 μM isoproterenol for 1 or 3 h. FIG. 4D shows the inhibition of β2AR degradation by FDPS inhibitor alendronate. Cells were treated with 0, 50, or 500 μM alendronate for 1 d, and then treated with 10 μM isoproterenol for 3 h. In both FIGS. 4C and 4D, cell surface β2AR was detected by flowcytometry using FITC-conjugated Flag antibody. MFI: Mean fluorescence intensity.

FIGS. 5A-5B show FDPS regulates the amount of Cell Surface β2AR in 293βAR^{WT} cells. FIG. 5A shows cells which were treated with 0 or 50 μM alendronate for 1 d, and then treated with 10 μM isoproterenol for 1 h. FIG. 5B shows cells which were treated with 0 or 100 μg/mL pravastatin for 1 d, and then treated with 10 μM isoproterenol for 1 h. Cell surface β2AR was detected by flowcytometry using FITC-conjugated Flag antibody. Error bars represented the standard deviations of replicates. Statistic analysis was using student's t-test. * p<0.05, MFI: Mean fluorescence intensity.

FIGS. 6A-6B show FDPS regulates the amount of Cell Surface β2AR in 293βAR^{WT} cells. FIG. 6A shows cells which were treated with control siRNA or FDPS siRNA in the presence or absence of 10 μM isoproterenol for 1 h, and in the presence or absence of cholesterol. FIG. 6B shows cells which were treated with methyl-beta cyclodextrin (MbCB), and then treated with 10 μM isoproterenol for 1 h. Cell surface β2AR was detected by flowcytometry using FITC-conjugated Flag antibody. Error bars represented the standard deviations of replicates. Statistic analysis was using student's t-test. * p<0.05, MFI: Mean fluorescence intensity.

FIGS. 7A-7F show that ARRDC3 is a β2-AR regulator gene which required for β2AR degradation and ubiquitination. FIG. 7A shows a schematic diagram of the RNAi -based screen to identify regulators of β2AR. FIG. 7B shows ARRDC3 knockdown enhances β2ARt membrane levels. FLAG-β2ARt cells transduced with lentiviral particles carrying a non-targeting (NT) shRNA or an ARRDC3-specific shRNA and treated as indicated before immunostaining and FACS analysis. Mean fluorescence intensities (MFI) of non-targeting (NT) or ARRDC3 shRNA-expressing cells are indicated. FIG. 7C shows the effects of siRNA-mediated ARRDC3 knockdown on β2ARt and FIG. 7D shows the effects of siRNA-mediated ARRDC3 knockdown on β2AR degradation. Normalized pixel densitometry values, shown as a bar graph, and standard errors (SE) are averages of three independent experiments; ** P<0.01 and *** P<0.001. FIG. 7E shows ubiquitination of β2ARt and FIG. 7F shows ubiquitination of β2AR in ARRDC1 and ARRDC3 knockdown cells. Results are representative of duplicate independent experiments of each (7E) and (7F).

FIGS. 8A-8E show ARRDC3 interacts with and recruits NEDD4 E3 ligase to the activated β2AR to mediate receptor ubiquitination. FIG. 8A shows the alignment of the PPXY-containing domains of ARRDC3 orthologs. PPXY motifs are highlighted in red in the alignment and were mutated as indicated to generate individual PPXY motif mutants ARRDC3-AASA and ARRDC3-AALA, or the double PPXY motif mutant ARRDC3ΔΔPPXY. FIG. 8B shows ARRDC3 interaction with NEDD4 requires the PPXY motifs. FIG. 8C shows colocalization of ARRDC3 with NEDD4. Transfected β2AR-expressing cells were treated as indicated and fixed with paraformaldehyde before visualization. White arrowheads indicate NEDD4 and ARRDC3 colocalization. FIG. 8D shows the effects of ARRDC3 or ARRDC3ΔΔPPXY expression on β2AR degradation. * indicates non-specific (NS) bands. FIG. 8E shows the effect of ARRDC3 or ARRDC3 PPXY mutant overexpression on β2AR ubiquitination. All results are representative of duplicate independent experiments.

FIGS. 9A-9B show ARRDC3 interacts more efficiently with activated β2AR. FIG. 9A shows the association of ARRDC3 with activated β2AR. Cells stably expressing FLAG-β2ARt were transfected with the indicated plasmids then subjected to a treatment with vehicle or ISO. Anti-FLAG immunoprecipitates from the corresponding lysates and whole cell extracts (WCE) were analyzed with the indicated antibodies. FIG. 9B shows ARRDC3 colocalizes with β2AR and EEA1 after agonist stimulation. β2AR-expressing cells transfected with GFP (control) or ARRDC3-GFP were incubated with a Rabbit raised anti-FLAG antibody and subjected to treatment with ISO (or vehicle) as indicated. Cells were fixed and permeabilized as detailed in the “Material Methods” section of the Examples, then incubated with a Mouse raised anti-EEA1 antibody. Cells were later stained with an anti-Rabbit IgG FITC-conjugated antibody and an anti-Mouse IgG Alexa 647-conjugated antibody before mounting on slides and visualization. Colocalization of β2AR (red) at the membrane and in early endosomal vesicles (EEA1; blue) with ARRDC3 (green) after stimulation with ISO. White arrowheads indicate regions of colocalization. All results are representative of duplicate independent experiments.

FIGS. 10A-10B show ARRDC3 is an essential adaptor for β2AR ubiquitination. FIG. 10A shows ARRDC3 is required for the NEDD4/β2AR association. Cells stably expressing FLAG-β2ARt were transfected with siRNAs targeting nothing (scrambled), ARRDC3, or β-arrestin-2 and then subjected to a treatment with vehicle or ISO. Anti-FLAG immunoprecipitates from the corresponding lysates and whole cell extracts (WCE) were analyzed by immunoblotting with the indicated antibodies. FIG. 10B is a schematic model of ARRDC3 functioning as an adaptor for β2AR ubiquitination.

FIG. 11A-11C show inhibition of an EST RNA (AA496068) increases latrophilin 2 expression and decreases β2-AR degradation. FIG. 11A show the genomic locus that is targeted by AA496068 EST shRNA (SEQ ID NO: 2). The shRNA corresponds to sequences in an EST AA496068, which is about 150 kb upstream of the latrophilin 2 gene, and binding of this shRNA TCTGCATTTCCTGTAATTTTGCATGC (SEQ ID NO: 2) activates latrophilin 2 and decreases β2-AR degradation. Thus SEQ ID NO: 2 serves as an activator or agonist of lactrophinin 2. The whole region covering the EST and latrophilin 2 is also annotated as KIAA0786. FIG. 11B shows use of shRNA of SEQ ID NO:2 to knockdown the nucleic acid of AA496068 EST to increase the amount of cell surface beta 2 adrenergic receptor. β2ARt-expressing 293 cells transfected with non-targeting (NT) or shRNA targeting the AA496068 EST were treated with vehicle or agonist (ISO) for 16 h. Cells were then labeled with anti-FLAG-FITC and analyzed by flow cytometry. FIG. 11B are histograms showing the distribution of cells treated with vehicle (upper panel) or ISO (lower panel) are shown above. An increase in the mean fluorescence intensity values in the shRNA-treated cells is shown. FIG. 11C shows the knockdown of AA496068 increases the expression of latrophilin 2. Total RNA was isolated from cells transfected with NT or AA496068 EST siRNAs. Latrophilin 2 and AA496068 EST expression was determined by reverse transcription using random hexamers followed by quantitative real-time PCR.

FIGS. 12A-12C show FDPS regulates β2AR expression and the elasticity of HSAM cells. FIG. 12A shows upregulation of β2AR by FDPS-specific shRNA. HSAM cells were transduced with microRNA—based lentiviral non targeting (NT) control shRNA or FDPS-specific shRNA. Cells were treated with 20 μM isoproterenol for 2 d and the total amount of β2AR was analyzed by immunoblots and densitometric measurement. Error bars represented the standard deviations; Student t-test * p<0.05, n=2. FIG. 12B shows subcellular localization of β2AR in FDPS knockdown HSAM cells. Indirect Immunofluorescence staining of β2AR and phase contract images are shown. FIG. 12C shows elastic modulus of control-transfected human airway smooth muscle cells (♦), and sample cells (O) measured versus time. The baseline elastic modulus of the FDPS knockdown cells (t=0) was approximately two times lower than the control cells. At t=60 s, cells were incubated in 1 mM isoproterenol and the elastic modulus of the cells was measured over 15 min as described in “Material and Methods” section in the Examples.

FIGS. 13A-13F show FDPS regulates the amount of Cell Surface β2AR through cholesterol independent mechanism. FIG. 13A shows acute cholesterol depletion inhibited β2AR down regulation. 293.β2AR* cells were treated with 1 or 2 mM MbCD for 1 h and followed by 10 μM isoproterenol (+Iso) for 1 h. Surface β2AR was determined by flowcytometry as above and relative mean fluorescence intensity (MFI) was shown (n=2). FIG. 13B shows the effect of alendronate (ALN) and pravastatin (PRAV) on cellular cholesterol content in serum containing medium. 293.β2AR* cells were cultured in medium containing 10% FBS until fully confluent and treated with 50 μM ALN or 100 μg/mL PRAN for 1 d, 2 mM MbCD or 2 mM cholesterol saturated MbCD (Chol-MbCD) for 1 h. Relative cholesterol contents were shown. Student t-test, ** p<0.005, *** p<0.001, n=4. FIG. 13C shows that Pravastatin treatment lowered cellular cholesterol content in serum free medium. 293.β2AR* cells were cultured in serum containing medium until fully confluent and treated with 50 μM ALN or 1, 10, 100 μg/mL PRVA for 1 d in serum free medium. Relative cholesterol contents were shown. Student t-test, ** p <0.005, *** p<0.001, n=4 or 8. FIGS. 13D and 13E show that Pravastatin treatment had no significant effect on Cell Surface β2AR. 293.β2AR* (see FIG. 13D) and 293.β2AR′ (FIG. 13E) cells were treated with 100 μg/mL PRVA for 1 d. Surface β2AR was determined by flowcytometry and relative MFI was shown (n=2). FIG. 13F shows that Pravastatin reversed the effect of alendronate on β2AR. 293.β2AR^{WT} cells were treated with 50 μM ALN alone or together with 1, 10, 100 μg/mL PRVA for 1 d. Surface β2AR was determined by flowcytometry and relative MFI was shown (n=2). Student t-test, ** p <0.005, *** p<0.001, n=2. Error bars represented the standard deviations of replicates.

FIGS. 14A-14C show primary human airway smooth muscle cells were transfected with non-targeting control siRNA or CaMKK2-specific siRNAs (#1, #2 or combination of 1 and 2; all are MISSION siRNAs from Sigma; at 50 nM). FIG. 14A shows β2AR and actin immunoblots three days after transfection of the cell lysates. FIG. 14B shows qRT-PCR to measure mRNA levels of CaMKK2, and FIG. 14C shows qRT-PCR to measure mRNA levels of β2AR.

FIG. 15A-15B show inhibition of CaMKK2 by small molecule inhibitor STO-609 increases β2AR protein level. FIG. 15A shows HASM cells, and FIG. 15B shows 293β2AR cells treated with the CaMKK inhibitor STO-609 at the indicated concentrations. Three days after the inhibitor treatment cell lysates were collected and analyzed by immunoblotting. Three repeats were done with similar results.

FIG. 16A-16B shows the effect of AAEST inhibition by siRNA on β2-adrenergic receptor expression. FIG. 16A shows shows surface β2-AR amount measured by FACS (α-Flag FITC) in HEK293 cells treated with different groups of siRNA targeting AAEST. FIG. 16B shows the total β2-AR protein measured by western blot in Human Airway Smooth Muscle (HASM) Cells treated with different groups of siRNA targeting AAEST.

FIG. 17 shows Cloning the full length Trasnscipt of AAEST. Confirmation of AAEST expression using RT-PCR with multiple primers targeting both predicted exon and intronic sequences. (negative controls with the absence of trasncripates are labeled −RT). The arrows on the schematic gene sequence below indicates the location of the primers in predicted exon and intron sequence of the AAEST region.

FIG. 18 is a schematic of the full length transcript of AAEST, illustrating the location of siRNA targets, and that the gene is 1.85 kb.

FIG. 19A-19B show the results from cloning of the full length transcript using 5′ and 3′ RACE reactions. FIG. 19A is the 5′ Race schematic (left panel) and results (right panel). FIG. 19B is the 3′ Race schematic (left panel) and results (right panel). (negative controls with the absence of transcriptase are labeled −RT). The arrows indicates sequence corresponding to AAEST region.

FIG. 20 shows β2-AR gene expression in the presence and absence of AAEST siRNA. Shown is a histogram of quantitative PCR results showing that siRNA inhibition does not decrease β2-AR receptor expression.

FIG. 21 is a schematic drawing showing a proposed mechanism that AAEST functions as a micro RNA (miRNA) which inhibits β2-AR expression at the protein level.

### DETAILED DESCRIPTION OF THE INVENTION

Using a unique genome-wide cell-based functional RNAi screen, the inventors have discovered over a dozen human genes that are critically required for agonist-induced beta-2 adrenergic receptor (β2-AR) downregulation. In particular, the inventors herein have discovered the role of these β2-AR regulator genes and their ability to prevent β2-AR downregulation in response to β2-AR agonist stimulation in human populations. One aspect of the invention relates to modulators of β2-AR regulator gene for use in the methods and compositions as disclosed herein for inhibiting β-AR downregulation in response to β2-AR agonist stimulation.

Accordingly, in some embodiments, modulators of β2-AR regulator gene are listed in Table 1, and includes for example, any one or more, or a combination of any selected from the following genes; FDPS (farnesyl diphosphate synthase), and/or ARRDC3 (arrestin domain containing 3) and/or CaMKK2, and/or AAEST and/or latrophilin 2.

In particular, one aspect of the present invention relates to inhibition of a β2-AR regulator gene to prevent β2-AR agonist-induced downregulation. Examples of such β2-AR regulator genes where inhibition is desired include FDPS (farnesyl diphosphate synthase), and/or ARRDC3 (arrestin domain containing 3) and/or CaMKK2 and/or AAEST.

Another aspect of the present invention relates to activation of a β2-AR regulator gene to prevent β2-AR agonist-induced downregulation. Examples of such β2-AR regulator genes where activation is desired include the latrophilin 2 gene.

Accordingly, in some embodiments the present invention also relates to methods to modulate (e.g. increase or decrease) the expression or function of a β2-AR regulator gene using RNAi or small molecules. Identification and characterization of these β2-AR regulator genes is not only an important contribution to the basic receptor biology, but also have the potential to contribute to the translational studies of β-agonist-based asthma therapy, as these regulators have been discovered to mediate the bronchodilator response in patients.

Other aspects of the present invention relate to methods for the treatment of a respiratory disorder, e.g. asthma, chronic obstructive pulmonary disease (COPD) and chronic bronchitis by co-administration of a β2-AR agonist and at least one modulator of a β2-AR regulator gene. In some embodiments, the β2-AR agonist can be administered to a subject with a respiratory disorder at the same time (e.g. simultaneously) or sequentially or separately with at least one modulator of a β2-AR regulator gene.

Other aspects of the present invention relate to admixtures comprising a β2-AR agonist and at least one modulator of a β2-AR regulator gene. In some embodiments, such an admixture can be used in methods for the treatment of a respiratory disorder in a subject, e.g. asthma and other respiratory disorders.

The inventors demonstrate herein the use of a novel EST-derived genome-wide RNAi library to identify genes regulating the agonist-induced internalization of β2AR, and the identification of several β2-AR regulator genes, as disclosed in Table 1. In one aspect of the invention, the inventors have demonstrated gene silencing RNAi agents which target the FDPS gene inhibited the internalization of β2AR and thus elevated the β2-AR protein level on cell surface. The inventors also demonstrate that small molecule inhibition of FDPS using alendronate, also inhibited β2-AR receptor internalization.

In one aspect of the invention, the inventors have also demonstrated that using gene activating RNAi agents (e.g. RNA effector molecules which activate gene expression) targeted to specific β2-AR regulator genes, e.g. latrophilin 2 gene, activate latrophilin 2 and inhibited the internalization of β2-AR and thus elevated the β2-AR protein level on cell surface. Thus, the inventors have demonstrated the utility of the EST-derived RNAi library as a tool for genome-wide screen to identify β2-AR regulator genes, and also demonstrate inhibition of specific β2-AR regulator genes (e.g. FDPS) or activation of specific β2-AR regulator genes (e.g. latrophilin 2) inhibit β2-AR receptor internalization.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “β2-AR” and “β2 Adrenergic receptor” and “beta 2 adrenergic receptor” are used interchangeably herein, and refer to the β2 adenosine receptor, also commonly known as alias ADRB2; ADRB2R; ADRBR; B2AR; BAR; BETA2AR by persons of ordinary skill in the art. By way of reference example only, the human mRNA for β2-AR is GenBank number NM_{—}000024, which encodes the human protein (amino acid) sequence for β2-AR which is GenBank No NP_{—}000015.

The term “β2-AR regulator gene” refers to any gene which modulates (e.g. increases or decreases) the internalization of the β2-AR, e.g. agonist-induced β2-AR internalization. Exemplary β2-AR regulator genes are described in Table 1 herein, and include for example, VAMP-A like Protein, Tubulin Polymerization-Promoting Protein Family Member 3 (TPPP3), Arrestin Domain Containing 3 (ARRDC3), latrophilin 2 (LPHN2), Guanine Nucleotide Binding Protein-like 3 (nucleolar)-like Protein, Dolichyl-Phosphate Mannosyltransferase Polypeptide 2, Farnesyl Diphosphate Synthase (FDPS), and Low Density Lipoprotein Receptor-related Protein 5, NEDD4, CAMKK2, PELP1, SNAPC5, GGT7, BZW1L1, BZW1L1 (pseudogene), GGN, GNL3L, NPIP and FBXO44. In some embodiments, a β2-AR regulator gene functions to increase internalization of the β2-AR, and thus inhibition of a β2-AR regulator gene decreases internalization of the β2-AR. Such β2-AR regulator gene wherein it is desirable to inhibit the β2-AR regulator gene, e.g. using an inhibitor of β2-AR regulator gene include, for example but are no way limited to, are Farnesyl Diphosphate Synthase (FDPS), Arrestin Domain Containing 3 (ARRDC3), CaMKK2, KIAA0786. In alternative embodiments, a β2-AR regulator gene functions to decrease the internalization of the β2-AR, and thus activation of a β2-AR regulator gene decreases internalization of the β2-AR. Such β2-AR regulator gene wherein it is desirable to activate the β2-AR regulator gene, for example, using an agonist, e.g. a RNA effector molecule which activates the β2-AR regulator gene include, but are no way limited to, latrophilin 2.

The term “modulator” refers to an agent which modules, e.g. increases or decreases the function of a gene or protein. Stated another way, the term “modulator” refers to an agent which increases or decreases the biological function of the molecule to which it is a modulator to. A modulator of a β2-AR regulator gene for example, refers to any agent or entity capable of inhibiting or activating the expression or biological activity of the β2-AR regulator gene. Thus, a modulator can operate to increase the transcription, translation, post-transcriptional or post-translational processing of the β2-AR regulator gene or otherwise activate the activity of the β2-AR regulator protein, polypeptide or polynucleotide encoded by the β2-AR regulator gene in any way. For example, in some embodiments, a modulator gene is an agent which inhibits β2-AR regulator gene (e.g. antagonists such as small molecules or gene silencing RNAi molecules which target and inhibit FDPS and/or ARRDC3 and/or CaMKK2 and/or AAEST) and in alternative embodiments, a modulator gene is an agent which activates a β2-AR regulator gene (e.g. agonists such as proteins, peptides or gene activating RNAi molecules which target and activate latrophilin 2).

In some embodiments, the modulation of a β2-AR regulator gene includes altering (e.g. increasing or decreasing) the expression of the β2-AR regulator gene, or level of mRNA molecule encoding a β2-AR regulator protein by at least 5%, 10%, 20%, 30%, 40%, 50%, 60%, 70%, 80%, 90%, 100%, 2-fold, 3-fold, 4-fold, 5-fold or more different from that observed in the absence of the modulator. The % and/or fold difference can be calculated relative to the control or the non-control, for example, the % difference (% difference) can be calculated by: dividing the level of mRNA expression in the presence of a modulator (x)—level of mRNA expression in the absence of the modulator (y) by either: the level of mRNA expression in the absence of the modulator (y) or the level of mRNA expression in the presence of a modulator (x).

The term “agonist” is well known in the art and can be used interchangeably herein with “activator” and refers to an agent or entity which activates or increases the biological function of the molecule to which it is an agonist to. An agonist of β2-AR refers to any agent or entity capable of activating the expression or biological activity of the β2-AR. Thus, an agonist can operate to increase the transcription, translation, post-transcriptional or post-translational processing or otherwise activate the activity of the protein, polypeptide or polynucleotide in any way, such as functioning as a ligand to activate a receptor or via other forms of direct or indirect action. By way of example only, an agonist which activates the β2-AR can be any entity or agent which functions as a ligand for β2-AR, such as a ligand which binds to the active site of the β2-AR, or alternatively any agent which interacts with the β2-AR (at the active site or at a non-active site) to initiate downstream signalling of the β2-AR. An agonist can be, for example a nucleic acid, peptide, or any other suitable chemical compound or molecule or any combination of these. In some embodiments, an agonist is an RNA such as a shRNA as disclosed herein, for example, a shRNA of SEQ ID NO: 2 which serves as an activator of the latrophilin 2 β2-AR regulator gene. Additionally, it will be understood that in indirectly activating the activity of a protein, polypeptide of polynucleotide, an agonist may affect the activity of the cellular molecules which may in turn act as regulators or the protein, polypeptide or polynucleotide itself. Similarly, an agonist may affect the activity of molecules which are themselves subject to the regulation or modulation by the protein, polypeptide of polynucleotide. An agonist can also be referred to herein as an “activating agent” or ‘activator”.

The term “antagonist” is well known in the art and can be used interchangeably herein with the term “inhibitor” and generally refers to an agent which inhibits or decreases the expression of a gene (e.g. a β2-AR regulator gene such as FDPS, ARRDC3, CaMKK2 or AAEST) or the biological function of the protein expressed by the gene by a statistically significant amount relative to in the absence of an inhibitor. The term “inhibition” or “inhibit” or “reduce” when referring to the activity of an agent which inhibits a β2-AR regulator gene as disclosed herein refers to prevention of β2-AR internalization and decreased β2-AR downregulation on β2-AR agonist stimulation. However, for avoidance of doubt, “inhibit” means statistically significant decrease in β2-AR downregulation on β2-AR agonist stimulation in the presence of an inhibitor of a β2-AR regulator gene by at least about 10% as compared to in the absence of the inhibitor of a β2-AR regulator gene, for example a decrease by at least about 20%, at least about 30%, at least about 40%, at least about 50%, or least about 60%, or least about 70%, or least about 80%, at least about 90% or more, up to and including a 100% of the downregulation of a β2-AR on β2-AR agonist stimulation, or any decrease in the downregulation or internalization of the β2-AR on β2-AR agonist stimulation between 10-100% as compared to in the absence an inhibitor of β2-AR regulator gene.

The terms “activate” or “increased” or “increase” as used in the context of biological activity of a protein herein generally means an increase in the biological function of the protein by a statically significant amount relative to in a control condition. For the avoidance of doubt, an “increase”, or “activation” of an β2-AR regulator gene (e.g. where the β2-AR regulator gene is latrophilin 2) means a statistically significant increase of at least about 10% of the activity of the β2-AR regulator gene (e.g. latrophilin 2) as compared to in the absence an activator agent, including an increase of at least about 20%, at least about 30%, at least about 40%, at least about 50%, at least about 60%, at least about 70%, at least about 80%, at least about 90%, at least about 100% or more, including, for example at least 2-fold, at least 3-fold, at least 4-fold, at least 5-fold, at least 10-fold increase or greater of activity or expression of the β2-AR regulator gene (e.g. latrophilin 2) as compared to the absence an activator agent.

The terms “increased”, “increase” or “enhance” or “higher” are all used herein to generally mean an increase by a statically significant amount; for the avoidance of any doubt, the terms “increased”, “increase” or “enhance” or “higher” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “lower”, “reduced”, “reduction” or “decrease” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The term “β2-AR agonist” or “β2 adrenergic receptor agonist” refers to any agent which increases the biological function of the β2-AR as compared to in the absence of the agonist. An agonist of β2-AR also refers to any agent or entity capable of activating the expression or biological activity of the β2-AR. Exemplary examples of β2-AR agonists include, Isoprenaline>epinephrine>>norepinephrine, where isoprenaline is more potent than epinephrine, which is more potent than noroepinephrine. Other examples of β2-AR agonists include, but are not limited to, Salbutamol (referred to also as Albuterol in USA), Bitolterol mesylate, Formoterol, Isoprenaline, Levalbuterol, Metaproterenol, Salmeterol, Terbutaline, Ritodrine.

The term “agent” refers to any entity which is normally not present or not present at the levels being administered in the cell. Agent can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc. A protein and/or peptide or fragment thereof can be any protein of interest, for example, but are not limited to: mutated proteins; therapeutic proteins and truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a β2-AR regulator gene within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, “gene silencing” or “gene silenced” in reference to an activity of an RNAi molecule, for example a siRNA or miRNA refers to a decrease in the mRNA level in a cell for a target gene (e.g. β2-AR regulator gene) by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, “gene activating” in reference to an activity of a RNAi molecule, for example a siRNA, shRNA or miRNA refers to an increase in the mRNA level in a cell for a target gene (e.g. β2-AR regulator gene) by at least about 5%, about 10%, about 20%, about 30%, about 40%, about 50%, about 60%, about 70%, about 80%, about 90%, about 95%, about 99%, about 100% of the mRNA level found in the cell without the presence of the shRNA, miRNA or RNAi molecule. In one preferred embodiment, the mRNA levels are increases by at least about 20% or at least about 30%, or at least about 40%, or at least about 50%, or at least about 60%, or at least about 70%, about 80%, about 90%, about 95%, about 99%, or about 100% or more than 100%, for example about 2-fold, or about 3-fold, or about 4-fold, or about 5-fold, or about 10-fold or about 20-fold, or about 50-fold, or about 100-fold or more than 100-fold, or any integer between about 20% to 100-fold increase in the mRNA level. Stated another way, a “gene activating RNAi” refers to a RNAi agent which upregulates or increases the expression or level or mRNA expression encoding a protein in the absence of a gene activating RNAi agent. The gene activating RNAi upregulates gene expression when the level of mRNA encoding the protein is increased by at least about 20% or at least about 30%, or at least about 40%, or at least about 50%, or at least about 60%, or at least about 70%, about 80%, about 90%, about 95%, about 99%, or about 100% or more than 100%, for example about 2-fold, or about 3-fold, or about 4-fold, or about 5-fold, or about 10-fold or about 20-fold, or about 50-fold, or about 100-fold or more than 100-fold, or any integer between about 20% to 100-fold increase in the mRNA level.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene such as a β2-AR regulator gene. By way of an example only, in some embodiments RNAi agents which serve to inhibit or gene silence are useful in the methods, kits and compositions disclosed herein to inhibit a β2-AR regulator gene, e.g. where the β2-AR regulator gene is, for example, FDPS and/or ARRDC3, and in alternative embodiments, RNAi agents which serve to activate the expression of genes are useful in the methods, kits and compositions disclosed herein to activate a β2-AR regulator gene, e.g. where the β2-AR regulator genes is, for example, latrophilin 2.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

The term “gene” used herein can be a genomic gene comprising transcriptional and/or translational regulatory sequences and/or a coding region and/or non-translated sequences (e.g., introns, 5′- and 3′-untranslated sequences and regulatory sequences). The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′- or 3′ untranslated sequences linked thereto. A gene can also be an amplified nucleic acid molecule produced in vitro comprising all or a part of the coding region and/or 5′- or 3′-untranslated sequences linked thereto.

The term “gene product(s)” as used herein refers to include RNA transcribed from a gene, or a polypeptide encoded by a gene or translated from RNA.

The term “nucleic acid” used herein refers to polynucleotides such as deoxyribonucleic acid (DNA), and, where appropriate, ribonucleic acid (RNA), polymers thereof in either single- or double-stranded form. Unless specifically limited, the term encompasses nucleic acids containing known analogs of natural nucleotides, which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19:5081 (1991); Ohtsuka, et al., J. Biol. Chem. 260:2605-2608 (1985), and Rossolini, et al., Mol. Cell. Probes 8:91-98 (1994)). The term nucleic acid is used interchangeably with gene, cDNA, and mRNA encoded by a gene. The term “nucleic acid” should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyedenosine, deoxycytidine, deoxyguanosine, and deoxythymidine. For purposes of clarity, when referring herein to a nucleotide of a nucleic acid, which can be DNA or RNA, the terms “adenosine”, “cytosine”, “guanosine”, and thymidine” are used. It is understood that if the nucleic acid is RNA, a nucleotide having a uracil base is uridine. The term “nucleotide” or nucleic acid as used herein is intended to refer to ribonucleotides, deoxyribonucleotides, acylic derivatives of nucleotides, and functional equivalents thereof, of any phosphorylation state. Functional equivalents of nucleotides are those that act as substrates for a polymerase as, for example, in an amplification method and artificial types of nucleic acids such as peptide nucleic acid (PNA) and locked nucleic acid (LNA) can be used. Functional equivalents of nucleotides are also those that can be formed into a polynucleotide that retains the ability to hybridize in a sequence specific manner to a target polynucleotide. As used herein, the term “polynucleotide” includes nucleotides of any number. A polynucleotide includes a nucleic acid molecule of any number of nucleotides including single-stranded RNA, DNA or complements thereof, double-stranded DNA or RNA, and the like.

The term “isolated” as used herein refers to the state of being substantially free of other material which is not the intended material. Stated another way, if the intended isolated product is a nucleic acid, the isolated nucleic acid is substantially free of other materials and/or contaminants such as proteins, lipids, carbohydrates, or other materials such as cellular debris or growth media. Typically, the term “isolated” is not intended to refer to a complete absence of these materials. Neither is the term “isolated” intended to refer the material is free from water, buffers, or salts, unless they are present in amounts that substantially interfere with the methods of the present invention. The term “isolated” as used herein when used with respect to nucleic acids, such as DNA or RNA, or proteins refers nucleic acids or peptides that are substantially free of cellular material, viral material, culture or suspension medium or chemical precursors or other chemical when isolated by the methods as disclosed herein. Moreover, an “isolated nucleic acid” is meant to include nucleic acid fragments which are not necessarily naturally occurring as fragments and can not typically be found in the natural state. Accordingly, an isolated nucleic acid encompass both an isolated heterologous and/or isolated recombinant nucleic acids. The term “isolated” as used herein can also refer to polypeptides which are isolated from other cellular materials and/or other proteins and is meant to encompass both purified and recombinant polypeptides.

The term “vector” used herein refers to a nucleic acid sequence containing an origin of replication. A vector can be a plasmid, bacteriophage, bacterial artificial chromosome or yeast artificial chromosome. A vector can be a DNA or RNA vector. A vector can be either a self replicating extrachromosomal vector or a vector which integrate into a host genome. The term “vectors” is used interchangeably with “plasmid” to refer to a nucleic acid molecule capable of transporting another nucleic acid to which it has been linked Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors can integrate into the host's genome or replicate autonomously in the particular cell. Other forms of expression vectors known by those skilled in the art which serve the equivalent functions can also be used. Expression vectors comprise expression vectors for stable or transient expression encoding the DNA.

The terms “polypeptide” and “protein” are be used interchangeably herein. A “peptide” is a relatively short polypeptide, typically between 2 and 60 amino acids in length, e.g., between 5 and 50 amino acids in length. Polypeptides (typically over 60 amino acids in length) and peptides described herein may be composed of standard amino acids (i.e., the 20 L-alpha-amino acids that are specified by the genetic code, optionally further including selenocysteine and/or pyrrolysine). Polypeptides and peptides may comprise one or more non-standard amino acids. Non-standard amino acids can be amino acids that are found in naturally occurring polypeptides, e.g., as a result of post-translational modification, and/or amino acids that are not found in naturally occurring polypeptides. Polypeptides and peptides may comprise one or more amino acid analogs known in the art can be used. Beta-amino acids or D-amino acids may be used. One or more of the amino acids in a polypeptide or peptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated may still be referred to as a “polypeptide”. Polypeptides may be purified from natural sources, produced in vitro or in vivo in suitable expression systems using recombinant DNA technology, synthesized through chemical means such as conventional solid phase peptide synthesis and/or using methods involving chemical ligation of synthesized peptides. The term “polypeptide sequence” or “peptide sequence” or “amino acid sequence” as used herein can refer to the polypeptide material itself or the peptide material itself and/or to the sequence information (i.e. the succession of letters or three letter codes used as abbreviations for amino acid names) that biochemically characterizes a polypeptide. Polypeptide sequences herein are presented in an N-terminal to C-terminal direction unless otherwise indicated.

The term “variant” as used herein refers to any polypeptide or peptide differing from a naturally occurring polypeptide by amino acid insertion(s), deletion(s), and/or substitution(s), created using, e.g., recombinant DNA techniques. In some embodiments, amino acid “substitutions” are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, tyrosine, asparagine, and glutamine. The positively charged (basic) amino acids include arginine, lysine and histidine. The negatively charged (acidic) amino acids include aspartic acid and glutamic acid. In some embodiments, cysteine is considered a non-polar amino acid. In some embodiments, insertions or deletions may range in size from about 1 to 20 amino acids, e.g., 1 to 10 amino acids. In some instances, larger domains may be removed without substantially affecting function. In certain embodiments, the sequence of a variant can be obtained by making no more than a total of 1, 2, 3, 5, 10, 15, or 20 amino acid additions, deletions, or substitutions to the sequence of a naturally occurring polypeptide. In some embodiments, not more than 1%, 5%, 10%, or 20% of the amino acids in a peptide, polypeptide or fragment thereof are insertions, deletions, or substitutions relative to the original polypeptide. In some embodiments, guidance in determining which amino acid residues may be replaced, added, or deleted without eliminating or substantially reducing activities of interest, may be obtained by comparing the sequence of the particular polypeptide with that of orthologous polypeptides from other organisms and avoiding sequence changes in regions of high conservation or by replacing amino acids with those found in orthologous sequences since amino acid residues that are conserved among various species may more likely be important for activity than amino acids that are not conserved.

The term “derivative” as used herein refers to peptides which have been chemically modified by techniques such as adding additional side chains, ubiquitination, labeling, pegylation (derivatization with polyethylene glycol), and insertion, deletion or substitution of amino acids, including insertion, deletion and substitution of amino acids and other molecules (such as amino acid mimetics or unnatural amino acids) that do not normally occur in the peptide sequence that is basis of the derivative, for example but not limited to insertion of ornithine which do not normally occur in human proteins. The term “derivative” is also intended to encompass all modified variants of the protein or peptides, variants, functional derivatives, analogues and fragments thereof, as well as peptides with substantial identity as compared to the reference peptide to which they refer to. The term derivative is also intended to encompass aptamers, peptidomimetics and retro-inverso peptides of the reference peptide to which they refer to. Amino acid substitutions include alterations in which an amino acid is replaced with a different naturally-occurring or a non-conventional amino acid residue. Such substitutions may be classified as “conservative”, in which case an amino acid residue contained in a polypeptide is replaced with another naturally occurring amino acid of similar character either in relation to polarity, side chain functionality or size.

Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments, amino acid substitutions are conservative.

As used herein, the terms “homologous” or “homologues” are used interchangeably, and when used to describe a polynucleotide or polypeptide, indicates that two polynucleotides or polypeptides, or designated sequences thereof, when optimally aligned and compared, for example using BLAST, version 2.2.14 with default parameters for an alignment (see herein) are identical, with appropriate nucleotide insertions or deletions or amino-acid insertions or deletions, in at least 70% of the nucleotides or amino acid residues, usually from about 75% to 99%, and more preferably at least about 98 to 99% of the nucleotides or amino acid residues. The term “homolog” or “homologous” as used herein also refers to homology with respect to structure and/or function. With respect to sequence homology, sequences are homologs if they are at least 50%, at least 60 at least 70%, at least 80%, at least 90%, at least 95% identical, at least 97% identical, or at least 99% identical. Determination of homologs of the genes or peptides of the present invention can be easily ascertained by the skilled artisan. Homologous sequences can be the same functional gene in different species.

The term “substantial identity” as used herein refers to two peptide sequences, when optimally aligned, such as by the programs GAP or BESTFIT using default gap weights, share at least about 65%, at least about . . . 70%, at least about . . . 80%, at least about . . . 90% sequence identity, at least about . . . 95% sequence identity or more (e.g., 99% sequence identity or higher). In some embodiments, residue positions which are not identical differ by conservative amino acid substitutions.

A “glycoprotein” as use herein is protein to which at least one carbohydrate chain (oligopolysaccharide) is covalently attached. A “proteoglycan” as used herein is a glycoprotein where at least one of the carbohydrate chains is a glycosaminoglycan, which is a long linear polymer of repeating disaccharides in which one member of the pair usually is a sugar acid (uronic acid) and the other is an amino sugar.

Substitutions encompassed by the present invention may also be “non conservative”, in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments, amino acid substitutions are conservative.

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e. without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Biological samples also include tissue biopsies, cell culture. A biological sample or tissue sample can refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the sample is from a resection, bronchoscopic biopsy, or core needle biopsy of a primary or metastatic tumor, or a cellblock from pleural fluid. In addition, fine needle aspirate samples are used. Samples may be either paraffin-embedded or frozen tissue. The sample can be obtained by removing a sample of cells from a subject, but can also be accomplished by using previously isolated cells (e.g. isolated by another person), or by performing the methods of the invention in vivo. Biological sample also refers to a sample of tissue or fluid isolated from an individual, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including but not limited to blood cells), tumors, organs, and also samples of in vitro cell culture constituent. In some embodiments, the biological samples can be prepared, for example biological samples may be fresh, fixed, frozen, or embedded in paraffin.

The term “tissue” is intended to include intact cells, blood, blood preparations such as plasma and serum, bones, joints, muscles, smooth muscles, and organs.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, interdisposition, affection. A disease and disorder, includes but is not limited to any condition manifested as one or more physical and/or psychological symptoms for which treatment is desirable, and includes previously and newly identified diseases and other disorders.

The term “basal cell” is a general term applied to any stratified or pseudostratified epithelium. It refers to cells which are juxtaposed to the basement membrane and under one or more additional epithelial layers. Many tissue can have both a two cell layer epithelium (basal and luminal cells) or a single layered epithelium. In the two cell layer, the cells adjacent to the basement membrane are termed “basal cells.”

The term “epitope” as used herein refers to that portion of an antigen that makes contact with a particular antibody.

The terms “respiratory disorder” and “respiratory disease” are used interchangeably herein and refer to any condition and/or disorder relating to respiration and/or the respiratory system. The respiratory disorder can be allergic or non-allergic. In some embodiments, the respiratory disorder is selected from the group consisting of asthma, atopic asthma, non-atopic asthma, emphysema, bronchitis, chronic obstructive pulmonary disease (COPD), sinusitis, allergic rhinitis. In some embodiments, the respiratory disorder is characterized by increased responsiveness of the tracheas and bronchi to various stimuli, i.e., allergens, resulting in a widespread narrowing of the airways.

The term “COPD” is generally applied to chronic respiratory disease processes characterized by the persistent obstruction of bronchial air flow. Typical COPD patients are those suffering from conditions such as bronchitis, cystic fibrosis, asthma or emphysema.

As used herein, the term “basal cell epitope” refers to an epitope that is present on the surface of the basal cell and whose expression is characteristic of basal cells.

The term “asthma” as used herein is defined as a disease of the airways that is characterized by increased responsiveness of the tracheobronchial tree to a multiplicity of stimuli.

The term “allergic respiratory disorder” or “hypersensitivity disease” refers to allergic diseases and/or disorders of the lungs or respiratory system. Allergic disorders are characterized by hypersensitivity to an allergen.

The term “atopic” as used herein refers to a state of atopy or allergy to an allergen or a state of hypersensitivity to an allergen. Typically, atopic refers to Type I hypersensitivity which results from release of mediators (e.g., histamine and/or leukotrines) from IgE-sensitized basophils and mast cells after contact with an antigen (allergen). An example of atopic is atopic asthma, which is allergic asthma and is characterized by an IgE response.

The term “allergen” as used herein refers to an innocuous antigen that induces an allergic or hypersensitive reaction.

The term “allergic rhinitis” as used herein is characterized by any of the following symptoms: obstruction of the nasal passages, conjuctival, nasal and pharyngeal itching, lacrimation, sneezing, or rhinorrhea. These symptoms usually occur in relationship to allergen exposure.

The term “non-allergic” as used herein refers to a respiratory disorder that is not a result from or caused by an allergen. Thus, the non-allergic respiratory disorder is 55 caused by other mechanisms not relating to hypersensitivity to air innocuous agent or allergen.

The term “treatment” refers to any treatment of a pathologic condition in a subject, particularly a human subject, and includes one or more of the following: (a) preventing a pathological condition from occurring in a subject which may be predisposition to the condition but has not yet been diagnosed with the condition and, accordingly, the treatment constitutes prophylactic treatment for the disease or condition; (b) inhibiting the pathological condition, i.e. arresting its development, (c) relieving the pathological condition, i.e. causing a regression of the pathological condition; or (d) relieving the conditions mediated by the pathological condition.

The pathological condition which is modulated by the treatment as disclosed herein, e.g. by a combination of β2-AR agonist and a modulator of a β2-AR regulator gene covers all disease states which are generally acknowledged in the art to be usefully treated with a β2-AR agonist in general. Such diseases states include, by way of example only, and are in no way limited to, asthma, chronic bronchitis, chronic pulmonary obstructive disease and the like.

The term “cells,” “host cells” or “recombinant host cells” are terms used interchangeably herein. It is understood that such terms refer not only to a particular cell type, but to the progeny or potential progeny of such a cell. Because certain modifications can occur in succeeding generations due to either mutation or environmental influences, such progeny can not, in fact, be identical to the parent cell, but are still included within the scope of the term as used herein.

The term “potency” refers to the minimum concentration at which an agent, e.g. β2-AR agonist is able to achieve a desirable biological or therapeutic effect. The potency of an agent, e.g. β2-AR agonist is typically proportional to its affinity for its ligand binding site. In some cases, the potency may be non-linearly correlated with its affinity. In comparing the potency of a drug under different situations, (e.g., the potency of a β2-AR agonist in the presence or absence of an inhibitor of a β2-AR regulator gene), the dose-response curve of each is determined under identical test conditions (e.g., in an in vitro or in vivo assay, in an appropriate animal model such a human patient). In some embodiments, an inhibitor of a β2-AR regulator gene can increase the potency of a β2-AR agonist. Stated another way, where a β2-AR agonist produces an equivalent biological or therapeutic effect at a lower concentration than in the absence of the inhibitor of a β2-AR regulator gene, it is indicative that the inhibitor of a β2-AR regulator gene has enhanced the potency of the β2-AR agonist.

The term “subject” refers to any living organism from which a biological sample can be obtained. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult and newborn subjects, as well as fetuses, whether male or female, are intended to be covered. The term “subject” is also intended to include living organisms susceptible to conditions or diseases caused or contributed bacteria, pathogens, disease states or conditions as generally disclosed, but not limited to, throughout this specification. Examples of subjects include humans, dogs, cats, cows, goats, and mice. The term subject is further intended to include transgenic species. In another embodiment, the subject is an experimental animal or animal substitute as a disease model.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Harper, N.J. (1962). Drug Latentiation in Jucker, ed. Progress in Drug Research, 4:221-294; Morozowich et al. (1977). Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977). Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997). Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996). Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metah. Pharmacokinet, 15(2): 143-53; Balimane and Sinko (1999). Involvement of multiple transporters in the oral absorption of nucleoside analogues, Adv. Drug Delivery Rev., 39(1-3):183-209; Browne (1997). Fosphenyloin (Cerebyx), Clin. Neuropharmacol. 20(1): 1-12; Bundgaard (1979). Bioreversible derivatization of drugs-principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996). Improved oral drug delivery: solubility limitations overcome by the use of prodrugs, Adv. Drug Delivery Rev. 19(2): 115-130; Fleisher et al. (1985). Design of prodrugs for improved gastrointestinal absorption by intestinal enzyme targeting, Methods Enzymol. 112: 360-81; Farquhar D, et al. (1983). Biologically Reversible Phosphate-Protective Groups, J. Pharm. Sci., 72(3): 324-325; Han, H. K. et al. (2000). Targeted prodrug design to optimize drug delivery, AAPS PharmSci., 2(1): E6; Sadzuka Y. (2000). Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl 2:S15-27; Wang, W. et al. (1999) Prodrug approaches to the improved delivery of peptide drugs. Curr. Pharm. Des., 5(4):265-87.

As used herein, a “pharmaceutical carrier” is a pharmaceutically acceptable solvent, suspending agent or vehicle for delivering an agent (e.g. agonist and/or antagonist) to the surface of a target cell, e.g. smooth muscle cell or basal cell or respiratory cell or to a subject. The carrier can be liquid or solid and is selected with the planned manner of administration in mind. Each carrier must be pharmaceutically “acceptable” in the sense of being compatible with other ingredients of the composition and non injurious to the subject.

The term “substantially pure”, with respect to nucleic acid refers to a sample comprising nucleic acids that is at least about 75%, preferably at least about 85%, more preferably at least about 90%, and most preferably at least about 95% pure, with respect to nucleic acids in the sample. Recast, the terms “substantially pure” or “essentially purified”, with regard to a preparation of a nucleic acid sample refer to a nucleic acid preparation that contains fewer than about 20%, more preferably fewer than about 15%, 10%, 8%, 7%, most preferably fewer than about 5%, 4%, 3%, 2%, 1%, or less than 1%, of non-nucleic acid molecules, such a proteins and other biomolecules.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “optional” or “optionally” means that the subsequent described event, circumstance or substituent may or may not occur, and that the description includes instances where the event or circumstance occurs and instances where it does not.

The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment of the invention.

Other than in the operating examples, or where otherwise indicated, all numbers expressing quantities of ingredients or reaction conditions used herein should be understood as modified in all instances by the term “about.” The term “about” when used in connection with percentages can mean±1%.

This invention is further illustrated by the examples which should not be construed as limiting. The contents of all references cited throughout this application, as well as the figures and tables are incorporated herein by reference.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### β2-Adrenergic Receptors

Adrenergic receptors (AR) are members of the G-protein coupled receptors that are composed of a family of three receptor sub-types: α1 (_{A, B, D}), α2 (_{A, B, C}), and β (_{1, 2, 3}). These receptors are expressed in tissues of various systems and organs of mammals and the proportions of the α and the β receptors are tissue dependant. For example, tissues of bronchial smooth muscle express largely β2-AR while those of cutaneous blood vessels contain exclusively α-AR subtypes.

It has been established that the β2-AR sub-type is involved in respiratory diseases such as such as asthma, chronic bronchitis, nervous system injury, and premature labor. Currently, a number of β2-AR agonists e.g., albuterol, formoterol, isoprenolol, or salmeterol having β2-AR agonist activities are being used to treat asthma. However, these drugs have limited utility as they are either non-selective thereby causing adverse side effects such as muscle tremor, tachycardia, palpitations, and restlesness, or have short duration of action and/or slow onset time of action. Accordingly, there is a need for methods to improve the duration of β2-AR agonists that have increased potency and/or longer duration of action.

The β2-adrenergic receptor, also referred to herein as β2AR or β2-AR is a prototypical G protein-coupled receptor (GPCR). Prolonged activation of the β2-AR receptor by β-agonists leads to β2-AR receptor degradation. While crucial for the receptor function, molecular mechanisms governing β2AR internalization and degradation remain incompletely defined. Disclosed herein describes the development of a novel EST-derived genome-wide RNAi (RNA interference) lentiviral library; its application in screening for genes regulating the agonist-induced internalization of β2AR; and identification of several novel β2AR regulator genes, including a key enzyme, farnesyl diphosphate synthase (FDPS) in the mevalonate pathway. Gene silencing using a gene silencing RNAi targeting the FDPS gene inhibited the internalization of β2AR and thus elevated the β2-AR protein level on cell surface. The inventors also demonstrate that small molecule inhibition of FDPS using alendronate, also inhibited β2-AR receptor internalization. The inventors have also demonstrated that using gene activating RNAi agents (e.g. RNA effector molecules which activate gene expression) targeted to specific β2-AR regulator genes, e.g. latrophilin 2 gene, activate latrophilin 2 and inhibited the internalization of β2-AR and thus elevated the β2-AR protein level on cell surface. Thus, the inventors have demonstrated the utility of the EST-derived RNAi library as a tool for genome-wide screen to identify β2-AR regulator genes, and also demonstrate inhibition of specific β2-AR regulator genes (e.g. FDPS) or activation of specific β2-AR regulator genes (e.g. latrophilin 2) inhibit β2-AR receptor internalization.

β2-AR is a subtype of β-adrenergic receptors, the classic seven transmembrane GPCRs and the important targets of the neurotransmitter catecholamines. Signaling from the receptor upon binding of an agonist triggers the sympathetic response widely in muscular, circulatory digestive and many other systems, for examples, relaxation of smooth muscles, contraction of heart muscles and glycogenolysis in liver. In cellular level, the immediate β2AR signaling is the activation of the receptor-associated adenylate cyclase and formation of cAMP. Further downstream signaling cascade includes activation of protein kinase A (PKA), opening of membrane ion channels and activation of many other biological pathways. Exposure to β-agonists leads to receptor internalization through endocytosis, which is a process known as receptor desensitization and an important mechanism for maintaining tissue homeostasis (Hanyaloglu, 2008; Moore, 2007). Regulation in trafficking (or sorting) of endosomes determines the fate of the internalized β2AR they have carried. Normally, most of these β2AR carrying endosomes recycle back to cell surface. However, significant amount of these endosomes would fuse with lysosomes and the receptors would undergo degradation if the exposure to agonists were longer than normal conditions. These could happen for example in long lasting β-agonist treatments. Although it is of physiological importance, such receptor regulation may contribute to the loss of drug potency or effectiveness (Tsao, 2000; Tsao, 2001). Recent studies had increased our understandings on regulation of receptor internalization and degradation (Marchese, 2008; Hanyaloglu, 2008; Moore, 2007).

### β2-AR Regulator Genes

The inventors herein have identified β2-AR regulator genes using a novel EST-derived shRNA library to perform a genome-wide screen for genes regulating β2-AR agonist-induced internalization of β2-AR. The inventors discovered at least 15 genes which function as β2-AR regulator genes which inhibit β2-AR agonist-induced β2AR internalization. The list of genes discovered are shown in Table 1. In particular, the inventors have discovered that farnesyl diphosphate synthase (FDPS), a key enzyme in the mevalonate pathway, and the pharmaceutical target for diseases of bone resorption, as a novel regulator in agonist-induced β2AR internalization.

TABLE 1
List of β-AR regulator genes.

| Gene | | siRNA | Gene | |
|---|---|---|---|---|
| Name | Description | Validation | Accession ID | Protein ID |
| ARRDC3 | Arrestin domain containing 3 | Yes | NM_020801 | NP_065852 |
| CaMKK2 | Calcium/calmodulin-dependent | Yes | NM_172226 | NP_757380 |
| | kinase kinase 2 | | | |
| FDPS | Farnesyl diphosphate synthase | Yes | NM_002004 | NP_001995 |
| KIAA0786 | Latrophilin 2 alternative splicing | Yes | NM_012302 | NP_036434 |
| | form (LEC1) | | | |
| LPHN2 | Latrophilin 2 | Yes | NM_012302 | NP_036434 |
| | | (activation) | | |
| TPPP3 | Tubulin polymerization- | n.d | NM_015964 | NP_057048 |
| | promoting protein 3 | | | |
| PELP1 | Proline, glutamate and leucine- | n.d | NM_014389 | NP_055204 |
| | rich protein 1 | | | |
| AI075761 | Transcribed locus Hs 52264 | n.d | | |
| SNAPCS | Small nuclear RNA activating | n.d. | NM_006049 | NP_006040.1 |
| | complex, polypeptide 5 | | | |
| FBXO44 | F-box protein 44 | n.d. | NM_183412 | NP_904319 |
| BZW1L1 | Basic leucine zipper and W2 | n.d. | NG_008827 | |
| | domain 1 like 1 (BZW1P1) | | | |
| GGN | gametogenetin | n.d. | NM_152657 | NP_689870 |
| GGT7 | Gamma glutamyltransferease 7 | n.d. | NM_178026 | NP_821158 |
| NPIP | Nuclear pore complex interacting | n.d. | NM_006985 | NP_008916 |
| | protein | | | |
| AI015265 | cDNA IMAGE clone 1641211 | n.d. | | |
| AAEST | | Yes | | |
| GNL3L | Guanine nucleotide binding | n.d. | NM_019067 | NP_061940 |
| | protein 3-like | | | |

(n.d refers to not determined)

### Farnesyl Diphosphate Synthase (FDPS)

The inventors have discovered Farnesyl diphosphate synthase (FDPS) (EC 2.5.1.10) is a β2-AR regulator gene, and demonstrate that inhibitors of FDPS decrease β2-AR internalization and increase β2-AR at the surface of the cell such as a basal cell. Thus, in some embodiments, the present invention is directed to a modulator of a β2-AR regulator gene which is an inhibitor (e.g. antagonist) of the FDPS β2-AR regulator gene. In some embodiments, a modulator of a β2-AR regulator gene which is any agent which inhibits FDPS, e.g. a small molecule inhibitor, gene silencing RNAi etc., are useful in the methods, compositions and kits as disclosed herein.

FDPS catalyzes the formation of both geranyl diphosphate and isopentenyl diphosphate from diphosphate and trans, trans-farnesyl diphosphate in the isoprene biosynthetic pathway. The human FDPS protein sequence has the database entry NP_{—}001995.1 (GI: 4503685). The nucleic acid sequence encoding human FDPS has the database entry NM_{—}002004.2 (GI: 41281370). The human FDPS gene is located at Iq22 and has the gene reference Gene ID: 2224 and the locus tags: HGNC: 3631 and MIM 134629. The sequence of the human FDPS gene is set out between bases 5769105-5780811 of the contig sequence GI: 51458934, NT_{—}004487.17 and between bases 5385993-5397702 of the contig sequence GI: 51460383, NT_{—}086596.1 (positions 152092649 and 152103528 on chromosome 1).

FDPS is a key branch point enzyme of the mevalonate pathway is farnesyl diphosphate synthase (FDPS, FPPS, EC 2.5.1.10), a Mg²⁺-dependent homodimeric enzyme, localized in peroxisomes. FDPS catalyzes the formation of both geranyl and farnesylpyrophosphate from isopentenyl pyrophosphate and dimethylallyl pyrophosphate. Post-translational modification of C-terminal CAAX sequences by covalent attachment of these isoprenyl chains is crucial for intracellular localization and proper function of small GTPases such as Ras, Rac, Rho, and CDC42.

Small Molecule Inhibitors of FDPS

Nitrogen-containing bisphosphonates (N-BPs) are known inhibitors of farnesyl diphosphate synthase and are currently used to treat osteoporosis, Paget's disease of the bone, and malignant bone tumors. Bisphosphonate therapy, which inhibits bone resorption, reduces the risk of fracture by 50% within one year. NE58025 is a research compound with 2 isomers. The 1R6S isomer projects the ring nitrogen towards the Lys200/Thr201 motif of the wildtype FPPS and is a potent inhibitor of FPPS, whereas its sister compound, the 1S6R isomer, projects the ring nitrogen away from the Lys200/Thr201 and is a poor inhibitor. An understanding of the mode of binding of NE58025 1R6S to an FDPS mutant that (Thr201Ala) adds to our understanding of the mechanism of inhibition of this important drug target.

In some embodiments, an inhibitor of FDPS is a bisphosphate. Bisphosphates (also referred to in the art as bis-phosphates) include, but are not limited to, adendronate, risedronate, eetidronate, zoledronate, clodronate, ibandronate, incadronate, medronate, neridronate, oxidronate, pamidronate or tiludronate. In some embodiment, a bisphosphate used as an inhibitor of a β2-AR regulator gene (e.g. inhibitor of FDPS) is adendronate.

The combined used of alendronate with calcitriol has been previously reported in International Application WO2001/028564 and European Patent Application No. 88-4628, discloses a pharmaceutical agent for oral administration which uses bisphosphonate as a substrate and contains an adequate amount of sodium lauryl sulfate, which are both incorporated herein by reference. However, alendronate has not been reported to be used in combination with a β2-AR agonist. In some embodiments, a subject is indentified to have a respiratory disorder as disclosed herein, or in need of β2-AR agonist therapy by a clinician before being administered a composition comprising alendronate and a β2-AR agonist, or being administered alendronate simultaneously, sequentially or separately with a β2-AR agonist.

Alendronate sodium is chemically known as (4-amino-1-hydroxybutylidene)bisphosphonic acid monosodium salt. Alendronate as disclosed in U.S. Pat. No. 4,621,077, which is incorporated herein by reference, is typically used as a specific inhibitor of osteoclast-mediated bone resorption and is indicated for the treatment of urolithiasis and inhibiting bone reabsorption.

Alendronate is commercially available as alendronate sodium trihydrate in the form of tablets and oral solution under the trade name FOSAMAX™ (Merck). Tablets have certain disadvantage for patients who are unable to swallow tablets readily. To overcome the problem of difficulty in swallowing, alendronate in solution formulation have been developed with an increased patient compliance. Commercially available alendronate oral solution contain 91.35 mg of alendronate monosodium salt trihydrate, which is molar equivalent to 70 mg of free acid alendronate as active ingredient and excipients such as sodium citrate dihydrate and citric acid anhydrous as buffering agents, sodium propylparaben 0.0225% and sodium butylparaben 0.0075% as preservatives, sodium saccharin, artificial raspberry flavor and purified water.

Given below are the patents/patent publications, which disclose aqueous oral formulations of alendronate. U.S. Pat. No. 4,814,326 which is incorporated herein by reference, discloses aqueous solution of diphosphonic acid with amino carboxylic acid as stabilizer with pH 4.5-5.65 which is incorporated herein by reference, U.S. Pat. No. 5,462,932 which is incorporated herein by reference, discloses a composition comprising a pharmaceutically effective amount of alendronate, in a pharmaceutically acceptable carrier and a sufficient amount of a buffer to maintain a pH of the composition in the range of 2 to 8 and complexing agent to prevent the precipitation of alendronate sodium in aqueous solution. U.S. Pat. No. 5,994,329, which is incorporated herein by reference, discloses liquid composition comprising alendronate monosodium trihydrate, sodium propylparaben, sodium butylparaben, sodium citrate dihydrate, citric acid anhydrous, sodium saccharin, sodium hydroxide and water. US 2003/0139378, which is incorporated herein by reference, discloses an oral liquid composition comprising: a) a therapeutically effective amount of at least one bisphosphonate or a pharmaceutically acceptable salt thereof, b) a pharmaceutically acceptable carrier, and c) a pharmaceutically acceptable buffer, wherein a dose of said oral liquid pharmaceutical composition has a buffering capacity sufficient to buffer at least 50 mL of 0.1 N HCl to a pH of greater than or equal to 3.5. US 2004/087550, which is incorporated herein by reference, discloses a composition for prevention of metabolic diseases of bones comprising: at least one bisphosphonate including alendronate sodium, viscosity agents comprising carboxymethylcellulose and xanthan gum; at least one flavoring agent and purified water. WO 98/14196, which is incorporated herein by reference, discloses an aqueous liquid formulation comprising: alendronic acid; a sufficient amount of a buffer such that the pH of the formulation is between approximately 3.5 and approximately 7.5 and 15 ml of the formulation is able to raise the pH of 50 ml 0.1N HCl to a pH of at least 3 and optionally, one or more additional agents selected from the group consisting of preservatives, flavoring agents, colorants, and sweeteners. WO 08/028,547, which is incorporated herein by reference, discloses liquid composition for prevention of bone metabolic diseases, comprising alendronic acid or its acceptable pharmaceutical salts, or mixtures thereof, a viscosity agent selected from the group consisting of alginate, propylglycolalginate, arabic gum (acacia), xanthan gum, guar gum, locust bean, carrageenan gum, karaya gum, tragacanth gum, chitosan, sodium carboxymethyl cellulose and carbomer or mixtures thereof, at least one flavoring agent and purified water. 2009/0029946, which is incorporated herein by reference, discloses an aqueous oral solution of bisphosphonic acid. U.S. Pat. No. 125,372, incorporated herein by reference also discloses enteric coated tablets that contain bisphosphonates.

In some embodiments, other non-limiting examples of small molecule inhibitors of FDPS include antibodies, ACETONEL™, isedronate, eetidronate, zoledronate (ZOMETA, ACLASTA), clodronate, ibandronate (BONIVA), incadronate, medronate, neridronate, oxidronate, pamidronate (APD, AREDIA), Risedronate (Actonel) or tiludronate.

Other Inhibitors of FDPS

In some embodiments, inhibitors of FDPS include neutralizing antibodies, including anti-FDPS antibodies which are commercially available. Some examples of commercially available anti-FDPS antibodies which can be used as a modulator to inhibit the FDPS β2-AR regulator gene according to the methods as disclosed herein include for example, but not limited to, Cell Signalling antibodies; Santa Cruz antibodies, and other commercial sources such as Cell Signalling, Invitrogen, Sigma, AdD Serotec and the like.

RNAi inhibitors of FPDS Inhibition of the FDPS β2-AR regulator gene can be by gene silencing RNAi molecules according to methods commonly known by a skilled artisan. For example, a gene silencing siRNA oligonucleotide duplexes targeted specifically to human FDPS (GenBank No: NM_{—}002004.2 (GI: 41281370) have been previously used to knockdown FDPS expression. FDPS mRNA has been successfully targeted using siRNAs; and other siRNA molecules may be readily prepared by those of skill in the art based on the known sequence of the target mRNA. To avoid doubt, the sequence of a human FDPS is provided at, for example, GenBank Accession Nos. NM_{—}002004.2 (GI: 41281370 RNAi agents are also commercially available, such as, for example, from Santa-Cruz Biotechnology) and also from other companies, such as Invitrogen.

### ARRDC3

The inventors have discovered ARRDC3, also known as arrestin domain containing 3 is a β2-AR regulator gene. The inventors have discovered that inhibitors of ARRDC3 decrease β2-AR internalization and increase β2-AR at the surface of the cell, such as e.g., a basal cell. Thus, in some embodiments, the present invention is directed to a modulator of a β2-AR regulator gene which is an inhibitor (e.g. antagonist) of the ARRDC3 β2-AR regulator gene. In some embodiments, a modulator of a β2-AR regulator gene which is any agent which inhibits ARRDC3, e.g. a small molecule inhibitor, gene silencing RNAi etc., are useful in the methods, compositions and kits as disclosed herein.

ARRDC3 is a member of the arrestins family of a conserved two-step mechanism for regulating the activity of G protein-coupled receptors (GPCRs). In response to a stimulus, GPCRs activate heterotrimeric G proteins. In order to turn off this response, or adapt to a persistent stimulus, activated receptors need to be silenced. The first step is phosphorylation by a class of serine/threonine kinases called G protein coupled receptor kinases (GRKs). GRK phosphorylation specifically prepares the activated receptor for arrestin binding. Arrestin binding to the receptor blocks further G protein-mediated signaling, targets receptor for internalization, and redirects signaling to alternative G protein-independent pathways. In addition to GPCRs, arrestins bind to other classes of cell surface receptors and a variety of other signaling proteins.

As used herein, the term “ARRDC3” refers to the nucleic acid encoding arrestin domain containing 3 gene and homologues thereof, including conservative substitutions, additions, deletions therein not adversely affecting the structure of function. ARRDC3 is referred in the art under synonyms Arrestin domain-containing protein 3, KIAA1376, TBP-2-like inducible membrane protein, TLIMP. Human ARRDC3 is encoded by nucleic acid corresponding to GenBank Accession No: NM_{—}020801.2 (GI: 195539387) and the human ADDRDC3 corresponds to protein sequence corresponding to GenBank ID No: NP_{—}065852.1, (Gene ID: 32698736).

A modulator which is an inhibitor of ARRDC3 can be any agent which inhibits the function of ARRDC3, such as antibodies, gene silencing RNAi molecules and the like. Commercial neutralizing antibodies of ARRDC3 are known to one of ordinary skill in the art, and include rabbit Anti-ARRDC3 Polyclonal Antibody, Unconjugated, from Abcam, Rabbit Anti-ARRDC3 Polyclonal Antibody, Clone C-14, from Santa Cruz Biotechnology, Inc; Rabbit Anti-ARRDC3 Polyclonal Antibody, Clone E-14, from Santa Cruz Biotechnology, Inc.

CaMKK2

The inventors have discovered CaMKK2 is a β2-AR regulator gene, and demonstrate that inhibitors of CaMKK2 decrease β2-AR internalization and increase β2-AR at the surface of the cell such as a basal cell. Thus, in some embodiments, the present invention is directed to a modulator of a β2-AR regulator gene which is an inhibitor (e.g. antagonist) of the CaMKK2 β2-AR regulator gene. In some embodiments, a modulator of a β2-AR regulator gene which is any agent which inhibits CaMKK2, e.g. a small molecule inhibitor, gene silencing RNAi etc., are useful in the methods, compositions and kits as disclosed herein.

As used herein, the term “CaMKK2” refers to the nucleic acid encoding calcium/calmodulin-dependent protein kinase kinase 2, beta (CAMKK2) gene and homologues thereof, including conservative substitutions, additions, deletions therein not adversely affecting the structure of function. CaMKK2 is referred in the art under synonyms CAMKK, KIAA0787, CAMKKB, MGC15254, calcium/calmodulin-dependent protein kinase kinase 2, beta, CaM-kinase kinase beta, CAMKKB, CaM-KK beta, CaM-kinase kinase, CAMKK, calcium/calmodulin-dependent protein kinase kinase, calcium/calmodulin-dependent protein kinase beta2, CaMKK, CAMKK beta protein, Calcium/calmodulin-dependent protein kinase kinase beta, CaMKK beta, CaM-KK. Human CaMKK2 is encoded by nucleic acid corresponding to GenBank Accession No: NM_{—}172226.2 (GI: 259490264) and the human CaMKK2 corresponds to protein sequence corresponding to GenBank ID No: NP_{—}757380.1, (Gene ID: 10645).

A modulator which is an inhibitor of CaMKK2 can be any agent which inhibits the function of CaMKK2, such as antibodies, gene silencing RNAi molecules and the like. Commercial neutralizing antibodies of CaMKK2 are known to one of ordinary skill in the art, and are encompassed for use in the methods and compositions as disclosed herein. Additionally, small molecules agonists of CaMKK2 are known by one of ordinary skill in the art and are encompassed for use in the methods and compositions as disclosed herein as an inhibitor of the CaMKK2 β2-AR regulator gene, for example, such small molecule inhibitor as disclosed in U.S. Patent Application US2010/0285033 which is incorporated herein in its entirety by reference. Other inhibitors of CaMKK2 are for example, but not limited to STO-609, a selective CaMKK2 inhibitor, CaMKII inhibitors such as KN62, KN93, and peptides derived from the auto-inhibitory region of CaMKII, such as AIP or ACS-I, are useful tools for examining functions of the kinase.

AAEST

The inventors have discovered AAEST is a β2-AR regulator gene, and demonstrate that inhibitors of AAEST decrease β2-AR internalization and increase β2-AR at the surface of the cell such as a basal cell. Thus, in some embodiments, the present invention is directed to a modulator of a β2-AR regulator gene which is an inhibitor (e.g. antagonist) of the AAEST β2-AR regulator gene. In some embodiments, a modulator of a β2-AR regulator gene which is any agent which inhibits AAEST, e.g. a small molecule inhibitor, gene silencing RNAi etc., are useful in the methods, compositions and kits as disclosed herein.

As used herein, the term “AAEST” refers to the nucleic acid of SEQ ID NO: 36 as disclosed herein, and homologues thereof, including conservative substitutions, additions, deletions therein not adversely affecting the structure of function.

The nucleic acid sequence for human AAEST transcript (SEQ ID NO: 36) is as follows:

| | |
|---|---|
| gatgaata actccacagc tcctcctgga | 81970583 |
| ccctgcgcgg gagcaggcgg ctcctgtgct gtaaagaaaa ttgattcgct | 81970633 |
| tgcggctcac ctcagtcgag gaagccctgg aatgttcagc agaacaccac | 81970683 |
| cactgtgaca tggggctgtg gcagtgggag acaccgcatg tggcgtgggt | 81970733 |
| gtgtgtggcg gcgtggctcg cactcagtcc tgtgtaggag aggaaaggga | 81970783 |
| ATCAAAAGCT TGAGCACAAA CAGATACCTC AGCCCGgtaa gctgcagctc | 81970833 |
| tgctcaactg cgtcttctct cagccctcca cacacgctca cccccactcc | 81970883 |
| cacacacaca cacacacaca cacacacaca ccatctcccg agggctgacc | 81970933 |
| tcctctggcc tcagcctgca gggtgtgggc agagaagggc atctgggacg | 81970983 |
| tggtgccagt gaggagccca gttggctggc actgggccca tttgaaggtg | 81971033 |
| tctcagacat ttggccagta tgtctttctc aggggtttgg tcacaaagga | 81971083 |
| tggactcttc ccacccagag gatgcaggga aagcacactg tgtctttccg | 81971133 |
| gtcattggat cctctccctt tccccaggca gctcgcctgg ccacaccgtt | 81971183 |
| ggagtgaacc ctcactgccc tcaaggacaa cagcagggtg tcacccagag | 81971233 |
| ccggatgagg gatgccagca ggtgccccca cgagtggggc cttggcccaa | 81971283 |
| gcagagcttc ccctgaaggt gccatcagcc agggcagctc tgtcccctcc | 81971333 |
| tgctctccat cttatatgta ttctaaccag gaaaaatgtg atagcacatg | 81971383 |
| ggtagcctag gcagtgaata aatacctcag atgtcctcct gcaaaaaaaa | 81971433 |
| aaaaaaaaaa aaaaatagga ggctgaaacc tagaactgag aaaaatctga | 81971483 |
| gtttttatta aaaaaaagca cgtttttact ttctgatatc cacctcagct | 81971533 |
| tttgttcttt aaaatgggat caatgtcatt acacaatttt cattaaaatc | 81971583 |
| atgtaaaaag caccacgctg tgcaaaagat gggcccaaat actctgcaaa | 81971633 |
| gatcattgca cgtaaatcag atcctttccc tctacctgta gGAGTTTCTG | 81971683 |
| TTCCTGTTCT TGAAGAGACA GACTGGTGAG CATGCAAAAT TACAGGAAAT | 81971733 |
| GCAGAGAACA AAATGGGCAG AGCAACCAAA ACTGTGGTGT TTGCATCAAA | 81971783 |
| TACAGGTCAA GAGTAAACTT ATTTTCCTAT GAAATTCAAG AACATGTTGA | 81971833 |
| AACTGGAAAG AGCGGGACAG GCTGGTAGCA CCTTTTAAAG ACCAAGAGAG | 81971883 |
| GCCGCCTCAT TAAATATTAA GAACTTGGAG GAAAGAGGTG GATTTACACT | 81971933 |
| GATAAAAGGT TCATTTAAAA TTCCATGAGG TCAATAAATT ACCACTTAag | 81971983 |
| atgccatttc ccaaaatgtg tcctgaagaa tgcttgtttt aaatgagggt | 81972033 |
| GGAGGGGTAG AGGGAAAAAA TCCTGTGGTC CAATTGATTT ATGCACTGTA | 81972083 |
| TCTCAGATGG AGTTTGACAA GAAATGTTGG CCAGGCGCGG TGTATCAAAC | 81972133 |
| CTGTAATCCC AGCACTTTGG GAGGCTGAGG TGGGAAGGAT CACTTGAGAC | 81972183 |
| CAGGAGTTTG AGACCAGCCT GGATGACATA AGGAGACCCC ATCTCTACAA | 81972233 |
| ATAATTAAAA AATTAGCCAG GTGTGTCAGT GCACACCTGT GGTCTCAGCT | 81972283 |
| ACTCAGGAGG CTGAGGCAAG AGGATCACCT AAGGCCAGGA GGTTGAGGCT | 81972333 |
| GCAGTGAGAT GTGATGGCAC CACTGCACTC CAGCCTGGGT GACTGAGTGA | 81972383 |
| GACCCAGTCT CAAAAGAACA AAACAAAACA AAACAAAACA AAACAAAACA | 81972433 |
| AAACAAAAAA CCAGACAATA AATTGTTGAG TTACCAAAGG CCCTGTTAAC | 81972483 |
| GCCTGCAACA AAAAAGATTG CTGAATCTTG CTTAGTCCAG TCTTTTCCAA | 81972533 |
| ACTTGTTTGA CCTTCAACCC CTTTCCTCTT CCTCTGCACA TTCATCTACA | 81972583 |
| TAACAAATGT ACAGAGGGCC TCCTCCAGGC CAGTCACTGT TCTAGGTCCT | 81972633 |
| ggggattcag cagtgaagaa aacaaaactc tcccttggca gactgtgtaa | 81972683 |
| ttaggaggag acagaaaata aaccaataga taaatcaaac cagaa | |

A modulator which is an inhibitor of AAEST can be any agent which inhibits the function of AAEST, such as antibodies, gene silencing RNAi molecules and the like. Commercial neutralizing antibodies of AAEST are encompassed for use in the methods and compositions as disclosed herein. Additionally, small molecules agonists of AAEST are known by one of ordinary skill in the art and are encompassed for use in the methods and compositions as disclosed herein as an inhibitor of the AAEST β2-AR regulator gene.

### Latrophilin 2

The inventors have discovered Latrophilin-2 and KIAA0786 are β2-AR regulator genes. The inventors have demonstrated that activators of Latrophilin-2 or KIAA0786 decrease β2-AR internalization and increase β2-AR at the surface of the cell, e.g. a basal cell. Thus, in some embodiments, the present invention is directed to a modulator of a β2-AR regulator gene which is an activator (e.g. agonist) of the Latrophilin-2 β2-AR regulator gene or the KIAA0786 β2-AR regulator gene. In some embodiments, a modulator of a β2-AR regulator gene which is any agent which activates or upregulates latrophilin 2, e.g. a protein, protein fragment or peptide of latrophilin 2, a small molecule agonist, a gene activating RNAi molecule etc., are useful in the methods, compositions and kits as disclosed herein.

Latrophilin 2 is a protein that in humans is encoded by the LPHN2 gene. This gene encodes a member of the latrophilin subfamily of G-protein coupled receptors (GPCR). Latrophilins may function in both cell adhesion and signal transduction. In experiments with non-human species, endogenous proteolytic cleavage within a cysteine-rich GPS (G-protein-coupled-receptor proteolysis site) domain resulted in two subunits (a large extracellular N-terminal cell adhesion subunit and a subunit with substantial similarity to the secretin/calcitonin family of GPCRs) being non-covalently bound at the cell membrane. While several transcript variants have been described, the biological validity of only one has been determined.

As used herein, the term “Latrophilin 2” refers to the nucleic acid encoding arrestin domain containing 3 gene and homologues thereof, including conservative substitutions, additions, deletions therein not adversely affecting the structure of function. Latrophilin 2 is referred in the art under synonyms secretin-type GPCR (Latrophilin) polypeptide, LPHN2, latrophilin-2, LPHN2, CIRL2, CL2, LEC1, LPHH1, CIRL2, CL2, LEC1, LPHH1, OTTHUMP00000011441, OTTHUMP00000011444, calcium-independent alpha-latrotoxin receptor 2; latrophilin homolog 1, latrophilin homolog 2 (cow); lectomedin-1. Human Latrophilin 2 is encoded by nucleic acid corresponding to GenBank Accession No: NM_{—}012302 (GI: GI:57165356) and the human Latrophilin-2 corresponds to protein sequence corresponding to GenBank ID No: NP_{—}036434.1, (Gene ID: GI:6912464).

To avoid doubt, the sequence of a human latrophilin 2 cDNA is provided at, for example, GenBank Accession Nos. NM_{—}012302. The sequence of human latrophilin 2 is the following sequence: GenBank Accession No: NM_{—}012302 (GI: GI: 57165356).

| (SEQ ID NO: 1) | |
|---|---|
| 1 | cggcgaacag acgttctttc tcctccatgc agttacacaa aaggagggct acggaaacta |
| 61 | aaagtttcgg ggcctctggc tcggtgtgtg gagaaaagag aaaacctgga gacgggatat |
| 121 | gaagatcaat gatgcagact gatggttttg atgaagctgg gcatttataa ctagattcat |
| 181 | taaggaatac aaagaaaata cttaaaggga tcaataatgg tgtcttctgg ttgcagaatg |
| 241 | cgaagtctgt ggtttatcat tgtaatcagc ttcttaccaa atacagaagg tttcagcaga |
| 301 | gcagctttac catttgggct ggtgaggcga gaattatcct gtgaaggtta ttctatagat |
| 361 | ctgcgatgcc cgggcagtga tgtcatcatg attgagagcg ctaactatgg tcggacggat |
| 421 | gacaagattt gtgatgctga cccatttcag atggagaata cagactgcta cctccccgat |
| 481 | gccttcaaaa ttatgactca aaggtgcaac aatcgaacac agtgtatagt agttactggg |
| 541 | tcagatgtgt ttcctgatcc atgtcctgga acatacaaat accttgaagt ccaatatgaa |
| 601 | tgtgtccctt acatttttgt gtgtcctggg accttgaaag caattgtgga ctcaccatgt |
| 661 | atatatgaag ctgaacaaaa ggcgggtgct tggtgcaagg accctcttca ggctgcagat |
| 721 | aaaatttatt tcatgccctg gactccctat cgtaccgata ctttaataga atatgcttct |
| 781 | ttagaagatt tccaaaatag tcgccaaaca acaacatata aacttccaaa tcgagtagat |
| 841 | ggtactggat ttgtggtgta tgatggtgct gtcttcttta acaaagaaag aacgaggaat |
| 901 | attgtgaaat ttgacttgag gactagaatt aagagtggcg aggccataat taactatgcc |
| 961 | aactaccatg atacctcacc atacagatgg ggaggaaaga ctgatatcga cctagcagtt |
| 1021 | gatgaaaatg gtttatgggt catttacgcc actgaacaga acaatggaat gatagttatt |
| 1081 | agccagctga atccatacac tcttcgattt gaagcaacgt gggagactgt atacgacaaa |
| 1141 | cgtgccgcat caaatgcttt tatgatatgc ggagtcctct atgtggttag gtcagtttat |
| 1201 | caagacaatg aaagtgaaac aggcaagaac tcaattgatt acatttataa tacccgatta |
| 1261 | aaccgaggag aatatgtaga tgttcccttc cccaaccagt atcagtatat tgctgcagtg |
| 1321 | gattacaatc caagagataa ccaactttac gtgtggaaca ataacttcat tttacgatat |
| 1381 | tctctggagt ttggtccacc tgatcctgcc caagtgccta ccacagctgt gacaataact |
| 1441 | tcttcagctg agctgttcaa aaccataata tcaaccacaa gcactacttc acagaaaggc |
| 1501 | cccatgagca caactgtagc tggatcacag gaaggaagca aagggacaaa accacctcca |
| 1561 | gcagtttcta caaccaaaat tccacctata acaaatattt ttcccctgcc agagagattc |
| 1621 | tgtgaagcat tagactccaa ggggataaag tggcctcaga cacaaagggg aatgatggtt |
| 1681 | gaacgaccat gccctaaggg aacaagagga actgcctcat atctctgcat gatttccact |
| 1741 | ggaacatgga accctaaggg ccccgatctt agcaactgta cctcacactg ggtgaatcag |
| 1801 | ctggctcaga agatcagaag cggagaaaat gctgctagtc ttgccaatga actggctaaa |
| 1861 | cataccaaag ggccagtgtt tgctggggat gtaagttctt cagtgagatt gatggagcag |
| 1921 | ttggtggaca tccttgatgc acagctgcag gaactgaaac ctagtgaaaa agattcagct |
| 1981 | ggacggagtt ataacaaggc aattgttgac acagtggaca accttctgag acctgaagct |
| 2041 | ttggaatcat ggaaacatat gaattcttct gaacaagcac atactgcaac aatgttactc |
| 2101 | gatacattgg aagaaggagc ttttgtccta gctgacaatc ttttagaacc aacaagggtc |
| 2161 | tcaatgccca cagaaaatat tgtcctggaa gttgccgtac tcagtacaga aggacagatc |
| 2221 | caagacttta aatttcctct gggcatcaaa ggagcaggca gctcaatcca actgtccgca |
| 2281 | aataccgtca aacagaacag caggaatggg cttgcaaagt tggtgttcat catttaccgg |
| 2341 | agcctgggac agttccttag tacagaaaat gcaaccatta aactgggtgc tgattttatt |
| 2401 | ggtcgtaata gcaccattgc agtgaactct cacgtcattt cagtttcaat caataaagag |
| 2461 | tccagccgag tatacctgac tgatcctgtg ctttttaccc tgccacacat tgatcctgac |
| 2521 | aattatttca atgcaaactg ctccttctgg aactactcag agagaactat gatgggatat |
| 2581 | tggtctaccc agggctgcaa gctggttgac actaataaaa ctcgaacaac gtgtgcatgc |
| 2641 | agccacctaa ccaattttgc aattctcatg gcccacaggg aaattgcata taaagatggc |
| 2701 | gttcatgaat tacttcttac agtcatcacc tgggtgggaa ttgtcatttc ccttgtttgc |
| 2761 | ctggctatct gcatcttcac cttctgcttt ttccgtggcc tacagagtga ccgaaatact |
| 2821 | attcacaaga acctttgtat caaccttttc attgctgaat ttattttcct aataggcatt |
| 2881 | gataagacaa aatatgcgat tgcatgccca atatttgcag gacttctaca ctttttcttt |
| 2941 | ttggcagctt ttgcttggat gtgcctagaa ggtgtgcagc tctacctaat gttagttgaa |
| 3001 | gtttttgaaa gtgaatattc aaggaaaaaa tattactatg ttgctggtta cttgtttcct |
| 3061 | gccacagtgg ttggagtttc agctgctatt gactataaga gctatggaac agaaaaagct |
| 3121 | tgctggcttc atgttgataa ctactttata tggagcttca ttggacctgt taccttcatt |
| 3181 | attctgctaa atattatctt cttggtgatc acattgtgca aaatggtgaa gcattcaaac |
| 3241 | actttgaaac cagattctag caggttggaa aacattaagt cttgggtgct tggcgctttc |
| 3301 | gctcttctgt gtcttcttgg cctcacctgg tcctttgggt tgctttttat taatgaggag |
| 3361 | actattgtga tggcatatct cttcactata tttaatgctt tccagggagt gttcattttc |
| 3421 | atctttcact gtgctctcca aaagaaagta cgaaaagaat atggcaagtg cttcagacac |
| 3481 | tcatactgct gtggaggcct cccaactgag agtccccaca gttcagtgaa ggcatcaacc |
| 3541 | accagaacca gtgctcgcta ttcctctggc acacagagtc gtataagaag aatgtggaat |
| 3601 | gatactgtga gaaaacaatc agaatcttct tttatctcag gtgacatcaa tagcacttca |
| 3661 | acacttaatc aaggacattc actgaacaat gccagggata caagtgccat ggatactcta |
| 3721 | ccgctaaatg gtaattttaa caacagctac tcgctgcaca agggtgacta taatgacagc |
| 3781 | gtgcaagttg tggactgtgg actaagtctg aatgatactg cttttgagaa aatgatcatt |
| 3841 | tcagaattag tgcacaacaa cttacggggc agcagcaaga ctcacaacct cgagctcacg |
| 3901 | ctaccagtca aacctgtgat tggaggtagc agcagtgaag atgatgctat tgtggcagat |
| 3961 | gcttcatctt taatgcacag cgacaaccca gggctggagc tccatcacaa agaactcgag |
| 4021 | gcaccactta ttcctcagcg gactcactcc cttctgtacc aaccccagaa gaaagtgaag |
| 4081 | tccgagggaa ctgacagcta tgtctcccaa ctgacagcag aggctgaaga tcacctacag |
| 4141 | tcccccaaca gagactctct ttatacaagc atgcccaatc ttagagactc tccctatccg |
| 4201 | gagagcagcc ctgacatgga agaagacctc tctccctcca ggaggagtga gaatgaggac |
| 4261 | atttactata aaagcatgcc aaatcttgga gctggccatc agcttcagat gtgctaccag |
| 4321 | atcagcaggg gcaatagtga tggttatata atccccatta acaaagaagg gtgtattcca |
| 4381 | gaaggagatg ttagagaagg acaaatgcag ctggttacaa gtctttaatc atacagctaa |
| 4441 | ggaattccaa gggccacatg cgagtattaa taaataaaga caccattggc ctgacgcagc |
| 4501 | tccctcaaac tctgcttgaa gagatgactc ttgacctgtg gttctctggt gtaaaaaaga |
| 4561 | tgactgaacc ttgcagttct gtgaattttt ataaaacata caaaaacttt gtatatacac |
| 4621 | agagtatact aaagtgaatt atttgttaca aagaaaagag atgccagcca ggtattttaa |
| 4681 | gattctgctg ctgtttagag aaattgtgaa acaagcaaaa caaaactttc cagccatttt |
| 4741 | actgcagcag tctgtgaact aaatttgtaa atatggctgc accatttttg taggcctgca |
| 4801 | ttgtattata tacaagacgt aggctttaaa atcctgtggg acaaatttac tgtaccttac |
| 4861 | tattcctgac aagacttgga aaagcaggag agatattctg catcagtttg cagttcactg |
| 4921 | caaatctttt acattaaggc aaagattgaa aacatgctta accactagca atcaagccac |
| 4981 | aggccttatt tcatatgttt cctcaactgt acaatgaact attctcatga aaaatggcta |
| 5041 | aagaaattat attttgttct attgctaggg taaaataaat acatttgtgt ccaactgaaa |
| 5101 | tataattgtc attaaaataa ttttaaagag tgaagaaaat attgtgaaaa gctcttggtt |
| 5161 | gcacatgtta tgaaatgttt tttcttacac tttgtcatgg taagttctac tcattttcac |
| 5221 | ttcttttcca ctgtatacag tgttctgctt tgacaaagtt agtctttatt acttacattt |
| 5281 | aaatttctta ttgccaaaag aacgtgtttt atggggagaa acaaactctt tgaagccagt |
| 5341 | tatgtcatgc cttgcacaaa agtgatgaaa tctagaaaag attgtgtgtc acccctgttt |
| 5401 | attcttgaac agagggcaaa gagggcactg ggcacttctc acaaactttc tagtgaacaa |
| 5461 | aaggtgccta ttctttttta aaaaaataaa ataaaacata aatattactc ttccatattc |
| 5521 | cttctgccta tatttagtaa ttaatttatt ttatgataaa gttctaatga aatgtaaatt |
| 5581 | gtttcagcaa aattctgctt ttttttcatc cctttgtgta aacctgttaa taatgagccc |
| 5641 | atcactaata tccagtgtaa agtttaacac ggtttgacag taaataaatg tgaatttttt |
| 5701 | caagtaaaaa aaaaaaaaaa aaa |

In some embodiments a protein, or protein fragment or polypeptide of latrophilin 2 can be used as a modulator in the methods, compositions and kits as disclosed herein. In some embodiments, a protein or protein fragment may be a protein, peptide or protein fragment of at least 10 amino acid sequence of latrophilin 2 protein.

In some embodiments, a modulator of a β2-AR regulator gene which is any agent which activates or upregulates KIAA0786, e.g. a protein, protein fragment or peptide of the gene product of KIAA0786, a small molecule agonist, a gene activating RNAi molecule etc., are useful in the methods, compositions and kits as disclosed herein.

In alternative embodiments, a gene activating RNAi molecule of latrophilin 2 mRNA or KIAA0786 can be used as a modulator in the methods, compositions and kits as disclosed herein.

In one embodiment, an activating RNAi molecule is used herein to activate expression of a β2-AR regulator gene, e.g. latrophilin 2 can be designed to target the 5′ upstream region of a gene, e.g. a promoter region of the gene that expresses the polypeptide to be modified. Induction of polypeptide expression by targeting promoters induces a potent transcriptional activation of associated genes (see e.g., Li, L C et al., Proc. Natl. Acad. Sci. U.S.A. 103 (46): 17337-42 (2006); Janowski, B A et al., Nat. Chem. Biol. 3 (3): 166-73 (2007); Li, L C et al., Caister Academic Press (2008); Check, E. et al., Nature 448 (7156): 855-8 (2007); Huang V et al., PLoS One 5 (1): e8848 (2010)). RNA activation can be performed in human cells using synthetic dsRNAs termed activating RNAi molecules, also referred to as “small activating RNAs (saRNAs)” in the art or miRNAs. In some embodiments, an activating RNAi agent which is useful as a modulator of the latrophilin 2 β2-AR gene is SEQ ID NO: 2. In some embodiments an activating siRNA agent useful as a modulator of the latrophilin 2 β2-AR gene targets a portion of the nucleic acid sequence AA496068 (SEQ ID NO: 5), where AA496068 is an EST upstream (>200 kB) of LPHN2 having the following sequence:

| (SEQ ID NO: 5) | |
|---|---|
| ATCAAAAGCT TGAGCACAAA CAGATACCTC AGCCCGGAGT TTCTGTTCCT | 50 |
| GTTCTTGAAG AGACAGACTG GTGAGCATGC AAAATTACAG GAAATGCAGA | 100 |
| GAACAAAATG GGCAGAGCAA CCAAAACTGT GGTGTTTGCA TCAAATACAG | 150 |
| GTCAAGAGTA AACTTATTTT CCTATGAAAT TCAAGAACAT GTTGAAACTG | 200 |
| GAAAGAGCGG GACAGGCTGG TAGCACCTTT TAAAGACCAA GAGAGGCCGC | 250 |
| CTCATTAAAT ATTAAGAACT TGGAGGAAAG AGGTGGATTT ACACTGATAA | 300 |
| AAGGTTCATT TAAAATTCCA TGAGGTCAAT AAATTACCAC TTA | |

In some embodiments, a modulator of latrophilin 2 β2-AR regulator gene is a gene activating RNAi, such as, for example, the shRNA sequence for kiaa0786 (latrophilin 2, AA496068), which is TCTGCATTTCCTGTAATTTTGCATGC (SEQ ID NO: 26). In some embodiments, a modulator which is a gene activating RNAi of AA496068 (e.g. a 5′ upstream region of latrophilin 2) for activation of the latrophilin 2 β2-AR regulator gene is a siRNA having the sequence: UAAUAUUUAAUGAGGCGGCCU (SEQ ID NO: 27).

In one embodiment, a modulator of latrophilin β2-AR regulator gene is used to upregulate or increase the expression of the latrophilin polypeptide for at least 1 day, at least 2 days, at least 3 days, at least 4 days, at least 5-days, at least 6-days, at least 7 days, at least 8 days, at least 9 days, at least 10 days, at least 11 days, at least 12 days, at least 13 days, at least 14 days, at least 15 days, at least 20 days, at least 25 days, at least 30 days or more.

### Other Agent Inhibitors of β2-AR Regulator Genes in General

Agents in General which Function as Inhibitors of β2-AR Regulator Gene

In some embodiments, the present invention relates to modulators which inhibit a β2-AR regulator gene. In such embodiments, the β2-AR regulator genes where the modulators which are inhibitor are, for example, FDPS and/or ARRDC3 and/or CaMKK2. In some embodiments, inhibition is inhibition of nucleic acid transcripts encoding a β2-AR regulator gene, for example inhibition of messenger RNA (mRNA). In alternative embodiments, inhibition of β2-AR regulator gene is inhibition of the expression and/or inhibition of activity of the gene product of β2-AR regulator gene, for example the polypeptide or protein of β2-AR regulator gene, or isoforms thereof. As used herein, the term “gene product” refers to RNA transcribed from a gene, or a polypeptide encoded by a gene or translated from RNA.

In some embodiments, inhibition of β2-AR regulator gene is by an agent. One can use any agent, for example but are not limited to nucleic acids, nucleic acid analogues, peptides, phage, phagemids, polypeptides, peptidomimetics, ribosomes, aptamers, antibodies, small or large organic or inorganic molecules, or any combination thereof. In some embodiments, agents useful in methods of the present invention include agents that function as inhibitors of the expression of a β2-AR regulator gene, e.g. FDPS and/or ARRDC3 and/or CaMKK2, for example inhibitors of mRNA encoding a β2-AR regulator gene, e.g. FDPS and/or ARRDC3 and/or CaMKK2 and/or AAEST.

Agents useful in the methods as disclosed herein can also inhibit gene expression (i.e. suppress and/or repress the expression of the gene). Such agents are referred to in the art as “gene silencers” and are commonly known to those of ordinary skill in the art. Examples include, but are not limited to a nucleic acid sequence, for an RNA, DNA or nucleic acid analogue, and can be single or double stranded, and can be selected from a group comprising nucleic acid encoding a protein of interest, oligonucleotides, nucleic acids, nucleic acid analogues, for example but are not limited to peptide nucleic acid (PNA), pseudo-complementary PNA (pc-PNA), locked nucleic acids (LNA) and derivatives thereof etc. Nucleic acid agents also include, for example, but are not limited to nucleic acid sequences encoding proteins that act as transcriptional repressors, antisense molecules, ribozymes, small inhibitory nucleic acid sequences, for example but are not limited to RNAi, shRNAi, siRNA, micro RNAi (miRNA), antisense oligonucleotides, etc.

As used herein, agents useful in the method as inhibitors of β2-AR regulator gene expression and/or inhibition of β2-AR regulator gene protein function can be any type of entity, for example but are not limited to chemicals, nucleic acid sequences, nucleic acid analogues, proteins, peptides or fragments thereof. In some embodiments, the agent is any chemical, entity or moiety, including without limitation, synthetic and naturally-occurring non-proteinaceous entities. In certain embodiments the agent is a small molecule having a chemical moiety.

In alternative embodiments, agents useful in the methods as disclosed herein are proteins and/or peptides or fragment thereof, which inhibit the gene expression of β2-AR regulator gene or the function of the protein expressed from the β2-AR regulator gene. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof.

Alternatively, agents useful in the methods as disclosed herein as inhibitors of β2-AR regulator gene can be a chemicals, small molecule, large molecule or entity or moiety, including without limitation synthetic and naturally-occurring non-proteinaceous entities. In certain embodiments the agent is a small molecule having the chemical moieties as disclosed herein.

### Small Molecules

All of the applications set out in the above paragraphs are incorporated herein by reference. It is believed that any or all of the compounds disclosed in these documents are useful for treatment of respiratory diseases and disorders, including, for example, but are not limited to asthma. In some embodiments, one of ordinary skill in the art can use other agents as inhibitors of β2-AR regulator gene, for example antibodies, or RNAi are effective for the treatment or prevention of respiratory diseases or disorders as claimed herein. In some embodiments, agents inhibiting β2-AR regulator gene can be assessed in models to determine β2-AR internalization and/or β2-AR agonist induced degradation as disclosed herein. For example, one can use the in vitro assay as disclosed in the Examples herein, where β2-AR degradation is monitored in the presence and absence of inhibitors of β2-AR regulator genes by methods commonly known by persons in the art.

Nucleic Acid Inhibitors of β2-AR Regulator Gene.

In some embodiments, agents that inhibit a β2-AR regulator gene are nucleic acids. Nucleic acid inhibitors of β2-AR regulator gene include, for example, but not are limited to, RNA interference-inducing molecules, for example but are not limited to siRNA, dsRNA, stRNA, shRNA and modified versions thereof, where the RNA interference molecule silences the gene expression of a β2-AR regulator gene.

β2-AR regulator genes can also be inhibited by “gene silencing” methods commonly known by persons of ordinary skill in the art. In some embodiments, the nucleic acid inhibitor of β2-AR regulator gene is an anti-sense oligonucleic acid, or a nucleic acid analogue, for example but are not limited to DNA, RNA, peptide-nucleic acid (PNA), pseudo-complementary PNA (pc-PNA), or locked nucleic acid (LNA) and the like. In alternative embodiments, the nucleic acid is DNA or RNA, and nucleic acid analogues, for example PNA, pcPNA and LNA. A nucleic acid can be single or double stranded, and can be selected from a group comprising nucleic acid encoding a protein of interest, oligonucleotides, PNA, etc. Such nucleic acid sequences include, for example, but are not limited to, nucleic acid sequence encoding proteins that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

In some embodiments single-stranded RNA (ssRNA), a form of RNA endogenously found in eukaryotic cells can be used to form an RNAi molecule. Cellular ssRNA molecules include messenger RNAs (and the progenitor pre-messenger RNAs), small nuclear RNAs, small nucleolar RNAs, transfer RNAs and ribosomal RNAs. Double-stranded RNA (dsRNA) induces a size-dependent immune response such that dsRNA larger than 30 bp activates the interferon response, while shorter dsRNAs feed into the cell's endogenous RNA interference machinery downstream of the Dicer enzyme.

RNA interference (RNAi) provides a powerful approach for inhibiting the expression of selected target polypeptides. RNAi uses small interfering RNA (siRNA) duplexes that target the messenger RNA encoding the target polypeptide for selective degradation. siRNA-dependent post-transcriptional silencing of gene expression involves cutting the target messenger RNA molecule at a site guided by the siRNA.

RNA interference (RNAi) is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease can be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA can be chemically synthesized, can be produced by in vitro transcription, or can be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and can contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand can follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The target gene or sequence of the RNA interfering agent can be a cellular gene or genomic sequence, e.g. the β2-AR regulator gene sequence. An siRNA can be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target sequence.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al, Nature Biotechnology 6:635-637, 2003. In addition to expression profiling, one can also screen the potential target sequences for similar sequences in the sequence databases to identify potential sequences which can have off-target effects. For example, according to Jackson et al. (Id.) 15, or perhaps as few as 11 contiguous nucleotides of sequence identity are sufficient to direct silencing of non-targeted transcripts. Therefore, one can initially screen the proposed siRNAs to avoid potential off-target silencing using the sequence identity analysis by any known sequence comparison methods, such as BLAST.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. For example, siRNA containing D-arabinofuranosyl structures in place of the naturally-occurring D-ribonucleosides found in RNA can be used in RNAi molecules according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotidesmolecules similar to the oligonucleotides containing 2′-O-methyl ribose, arabinose and particularly D-arabinose (U.S. Pat. No. 5,177,196).

The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

siRNA and miRNA molecules having various “tails” covalently attached to either their 3′- or to their 5′-ends, or to both, are also known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Other siRNAs useful for targeting a β2-AR regulator gene can be readily designed and tested. Accordingly, siRNAs useful for the methods described herein include siRNA molecules of about 15 to about 40 or about 15 to about 28 nucleotides in length, which are homologous to the β2-AR regulator gene. Preferably, the β2-AR regulator gene targeting siRNA molecules have a length of about 25 to about 29 nucleotides. More preferably, the β2-AR regulator gene targeting siRNA molecules have a length of about 27, 28, 29, or 30 nucleotides. The β2-AR regulator gene targeting siRNA molecules can also comprise a 3′ hydroxyl group. The β2-AR regulator gene targeting siRNA molecules can be single-stranded or double stranded; such molecules can be blunt ended or comprise overhanging ends (e.g., 5′, 3′). In specific embodiments, the RNA molecule is double stranded and either blunt ended or comprises overhanging ends.

In one embodiment, at least one strand of the β2-AR regulator gene targeting RNA molecule has a 3′ overhang from about 0 to about 6 nucleotides (e.g., pyrimidine nucleotides, purine nucleotides) in length. In other embodiments, the 3′ overhang is from about 1 to about 5 nucleotides, from about 1 to about 3 nucleotides and from about 2 to about 4 nucleotides in length. In one embodiment the β2-AR regulator gene targeting RNA molecule is double stranded—one strand has a 3′ overhang and the other strand can be blunt-ended or have an overhang. In the embodiment in which the β2-AR regulator gene targeting RNA molecule is double stranded and both strands comprise an overhang, the length of the overhangs can be the same or different for each strand. In a particular embodiment, the RNA of the present invention comprises about 19, 20, 21, or 22 nucleotides which are paired and which have overhangs of from about 1 to about 3, particularly about 2, nucleotides on both 3′ ends of the RNA. In one embodiment, the 3′ overhangs can be stabilized against degradation. In a preferred embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

The β2-AR regulator genes as disclosed herein have successfully targeted using siRNAs as disclosed herein. For example, gene silencing RNAi of FDPS and/or ARRDC3 and/or CaMKK2 are commercially available, for example from Invitrogen. In some embodiments, gene silencing RNAi agents can be produced by one of ordinary skill in the art and according to the methods as disclosed herein. In some embodiments, the assessment of the expression and/or knock down of a β2-AR regulator gene, e.g. FDPS and/or ARRDC3 and/or CaMKK2 β2-AR regulator gene can be determined using commercially available kits known by persons of ordinary skill in the art. Others can be readily prepared by those of skill in the art based on the known sequence of the target mRNA.

To avoid doubt, the sequence of a human FDPS cDNA is provided at, for example, GenBank Accession Nos.: NM_{—}002004.2 (GI: 41281370) and can be used to design a gene silencing RNAi modulator which inhibits FDPS mRNA expression. The sequence of human FDPS is the following sequence:

| (SEQ ID NO: 3) | |
|---|---|
| 1 | ggaacaggat gcccctgtcc cgctggttga gatctgtggg ggtcttcctg ctgccagccc |
| 61 | cctactgggc accccgggag aggtggctgg gttccctacg gcggccctcc ctggtgcacg |
| 121 | ggtacccagt cctggcctgg cacagtgccc gctgctggtg ccaagcgtgg acagaggaac |
| 181 | ctcgagccct ttgctcctcc ctcagaatga acggagacca gaattcagat gtttatgccc |
| 241 | aagaaaagca ggatttcgtt cagcacttct cccagatcgt tagggtgctg actgaggatg |
| 301 | agatggggca cccagagata ggagatgcta ttgcccggct caaggaggtc ctggagtaca |
| 361 | atgccattgg aggcaagtat aaccggggtt tgacggtggt agtagcattc cgggagctgg |
| 421 | tggagccaag gaaacaggat gctgatagtc tccagcgggc ctggactgtg ggctggtgtg |
| 481 | tggaactgct gcaagctttc ttcctggtgg cagatgacat catggattca tcccttaccc |
| 541 | gccggggaca gatctgctgg tatcagaagc cgggcgtggg tttggatgcc atcaatgatg |
| 601 | ctaacctcct ggaagcatgt atctaccgcc tgctgaagct ctattgccgg gagcagccct |
| 661 | attacctgaa cctgatcgag ctcttcctgc agagttccta tcagactgag attgggcaga |
| 721 | ccctggacct cctcacagcc ccccagggca atgtggatct tgtcagattc actgaaaaga |
| 781 | ggtacaaatc tattgtcaag tacaagacag ctttctactc cttctacctt cctatagctg |
| 841 | cagccatgta catggcagga attgatggcg agaaggagca cgccaatgcc aagaagatcc |
| 901 | tgctggagat gggggagttc tttcagattc aggatgatta ccttgacctc tttggggacc |
| 961 | ccagtgtgac cggcaaaatt ggcactgaca tccaggacaa caaatgcagc tggctggtgg |
| 1021 | ttcagtgtct gcaacgggcc actccagaac agtaccagat cctgaaggaa aattacgggc |
| 1081 | agaaggaggc tgagaaagtg gcccgggtga aggcgctata tgaggagctg gatctgccag |
| 1141 | cagtgttctt gcaatatgag gaagacagtt acagccacat tatggctctc attgaacagt |
| 1201 | acgcagcacc cctgccccca gccgtctttc tggggcttgc gcgcaaaatc tacaagcgga |
| 1261 | gaaagtgacc tagagattgc aagggcgggg agaggaggct ctcaataaat aatcgtgtaa |
| 1321 | ccttaaaaaa aaaaaaaaaa aaaaa |

In some embodiments, a modulator which is a gene silencing RNAi agent which downregulates or decreases FDPS mRNA levels is a 25-nt hairpin sequence (5′-AGC GGA GAA AGT GAC CTA GAG ATT G-3′ (SEQ ID NO: 20) as disclosed herein in the Examples. In some embodiments, a modulator of FDPS β2-AR regulator gene is a gene silencing RNAi, such as, for example, a shRNA sequence which is: CAATCTCTAGGTCACTTTCTCCGCT (SEQ ID NO: 21). In some embodiments, a modulator which is a gene silencing RNAi of the FDPS β2-AR regulator gene is 5′-CCA UGU ACA UGG CAG GAA U(dT)(dT)-3′ (sense strand) (SEQ ID NO: 22), and 5′-AUU CCU GCC AUG UAC AUG G(dT)(dT)-3′(antisense strand) (SEQ ID NO: 23) which is commercially available, for example from Sigma-Aldrich or from Dharmacon.

To avoid doubt, the sequence of a human ARRDC3 cDNA is provided at, for example, GenBank Accession Nos.: NM_{—}020801.2 and can be used by one of ordinary skill in the art to design a gene silencing RNAi modulator which inhibits ARRDC3 mRNA expression. In some embodiments, a modulator of ARRDC3 β2-AR regulator gene is a gene silencing RNAi, such as, for example, a shRNA sequence which is CCACAGACACCACTCGCTACCTCATT (SEQ ID NO: 24). In some embodiments, a modulator for use in the methods, compositions and kits disclosed herein is a gene silencing RNAi of the ARRDC3 β2-AR regulator gene which is a siRNA having the sequence: GAACUGCUCUUCCCGAAUG (SEQ ID NO: 25). Alternatively, one can use any gene silencing siRNA which targets a region of the sequence of human ARRDC3, which as the following sequence: NM_{—}020801.2 (GI: 195539387):

| (SEQ ID NO: 4) | |
|---|---|
| 1 | agcttttagc cgcccgtctt tgtaaggaga ccactgagac gagcgggagc gcggagcagc |
| 61 | agcctctgct gccctgactt tttaagaaat ctcaatgaac tatttgtaga gaatcactga |
| 121 | tccggcctgc aagcattttg cacggcaaaa atatcgatca gtgttaagtg aagatcacat |
| 181 | tttatatgcg atcttgactt ttttgtctta cattatattt ttatagattt tgttataaac |
| 241 | atggtgctgg gaaaggtgaa gagtttgaca ataagctttg actgtcttaa tgacagcaat |
| 301 | gtccctgtgt attctagtgg ggataccgtc tcaggaaggg taaatttaga agttactggg |
| 361 | gaaatcagag taaaatctct taaaattcat gcaagaggac atgcgaaagt acgctggact |
| 421 | gaatctagaa acgccggctc caatactgcc tatacacaga attacactga agaagtagag |
| 481 | tatttcaacc ataaagacat cttaattggg cacgaaagag atgatgataa ttccgaagaa |
| 541 | ggcttccaca ctattcattc aggaaggcat gaatatgcat tcagcttcga gcttccacag |
| 601 | acaccactcg ctacctcatt cgaaggccga catggcagtg tgcgctattg ggtgaaagcc |
| 661 | gaattgcaca ggccttggct actaccagta aaattaaaga aggaatttac agtctttgag |
| 721 | catatagata tcaacactcc ttcattactg tcaccccaag caggcacaaa agaaaagaca |
| 781 | ctctgttgct ggttctgtac ctcaggccca atatccttaa gtgccaaaat tgaaaggaag |
| 841 | ggctataccc caggtgaatc aattcagata tttgctgaga ttgagaactg ctcttcccga |
| 901 | atggtggtgc caaaggcagc catttaccaa acacaggcct tctatgccaa agggaaaatg |
| 961 | aaggaagtaa aacagcttgt ggctaacttg cgtggggaat ccttatcatc tggaaagaca |
| 1021 | gagacgtgga atggcaagtt gctgaaaatt ccaccagttt ctccctctat cctcgactgt |
| 1081 | agtataatcc gcgtggaata ttcactaatg gtatatgtgg atattcctgg agctatggat |
| 1141 | ttatttctta atttgccact tgtcatcggt accattcctc tacatccatt tggtagcaga |
| 1201 | acctcaagtg taagcagtca gtgtagcatg aatatgaact ggctcagttt atcacttcct |
| 1261 | gaaagacctg aagcaccacc cagctatgca gaagtggtaa cagaggaaca aaggcggaac |
| 1321 | aatcttgcac cagtgagtgc ttgtgatgac tttgagagag cccttcaagg accactgttt |
| 1381 | gcatatatcc aggagtttcg attcttgcct ccacctcttt attcagagat tgatccaaat |
| 1441 | cctgatcagt cagcagatga tagaccatcc tgcccctctc gttgaaggaa cacttggttg |
| 1501 | aatcaagttg atgtgggttc cgaactgtat ctcttccggc tgaggacaga gaagtatctt |
| 1561 | ggagacacgt ttcagaggaa gtggaattac ttttgcccag aaaaatggcg aatacatgaa |
| 1621 | acaaccagtg atcatgcttt agaagcctac agcaacattc tgagactgct ccaacatgct |
| 1681 | tgaagatcta agcttttctc ttttaaaact ggcacatact cagagcagtc ttcttagcct |
| 1741 | atggtcgtac gtgtcaagac atcacgttgt aaagagggat gatttccttc ttttgatttg |
| 1801 | aaaatttgca catgctcaat gcttacattg tgcggttcga cgtcactaca gcttcttttt |
| 1861 | tttttttttt ttttttctat ttttgccaga ctcttgatac tcttaaaact tgtttgtggt |
| 1921 | cagcacaaca aggaacaaaa caaagctttg aaaaaacttt aacatgaaaa aacgcactga |
| 1981 | catttttttt tatttaatat agcctggact ttacctgcgt atgcacatgc tcagaattgt |
| 2041 | ctactaggct gactatgtat cacctcttca gcttggatcc aattgtggat ttatttacaa |
| 2101 | acatcaaatg ccttcaagcc aatccttttt gctgtatgtt ttgcagccta ctgtagtaga |
| 2161 | tacgcaacag ataatgtggg aaaaaaagag ataagaggag gaagctaata agagactgtc |
| 2221 | aagattgtat accttcttgg tttcttttaa gaatttgttg cctttctact attacagcaa |
| 2281 | agcagcattt tgttactgac tgcctaaaat cacttaatct caggtgaacg catcacttgc |
| 2341 | caaactgttg gaatgctatt tgtgttttgt tgcactgttt ttttcgtttg tttgtttgtt |
| 2401 | tatttggttg gctttttgga gagggaaatt tggaaacggg acatacacaa aagttacaca |
| 2461 | cccacattcc ctttttatca tgacatacaa gaagaaacta gcagagctaa gaatggagtg |
| 2521 | aagaaaggca gtatggcagg caccagcaaa gagttgaggg ctgttgctct taaaaattat |
| 2581 | tttttttatt attattttga aagtatggaa gttttccatt cactggggaa aggagggaaa |
| 2641 | agtgcattta tttttataca gagttactta attacctcca aaacacatat gttggaaatc |
| 2701 | gcttttgctg gtgcaaagta tattaatgag caggaataca tacattgagg ttatgaatag |
| 2761 | agagctcaat ttgtaccttt gctgtcttgc tcaagcttgg tatggcatga aaactcgact |
| 2821 | ttattccaaa agtaacttca aaatttaaaa tactagaacg tttgctgcga taaatctttt |
| 2881 | ggatttttgt gtttttctaa tgagaatact gtttttcatt acctaaagaa caatttgcta |
| 2941 | aacatgagaa atcactcact ttgattatgt atagattaca taggaagaac aatcacatca |
| 3001 | gtaagttata gtttatatta aaggtaattt tctgttggct cataacaaat ataccagcat |
| 3061 | tcatgatagc atttcagcat tttccaaggt accaagtgta cttattttgt tgttgttgtt |
| 3121 | gttgttgtat tttagaagga attcagctct gatgttttta aagaaaacca gcatctctga |
| 3181 | tgttgcaaca tacgtgtaaa atgggtgtta catctatcct gccatttaac cccacagtta |
| 3241 | ataaagtggc tgaaaataat agtagctctg gcttggtgct tgacctggtt aaatactgtc |
| 3301 | ttaaagctca tacaaaacaa ataggctttt ccataagtgg cctttaagaa aacatggaag |
| 3361 | acaattcatg tttgacaaat gctgacaggg tgaagaaagc ccagtgtaaa aatgaatcgc |
| 3421 | gttttaagtg attcggttaa agagtttggg ctcccgtagc aaactaatac tagataataa |
| 3481 | ggaaatgggg gtgaaatatt tttttattgt tgaatcattt tgtgaatgtc cccctcaaaa |
| 3541 | aaagctaatg gaatatttgg cataaagggc atttggtggt tttatttttg tttgaggggg |
| 3601 | attgtcagaa aatccctttt ctctcttacg tctaactgac tagggaacaa ttgttgatat |
| 3661 | gcatagcatt ggaatacttg tcattatata ctcttacaaa taacacatga agcaagaatg |
| 3721 | accaatattc tgataattgg cactggatca caaaatgtga taaaacttta aatgtataaa |
| 3781 | actttatcaa ataaagtttt attttcccct ttaaaatgta tttctttaga ggcattactt |
| 3841 | ttttaaaaat attggtcaat tcctgacata agatgtgagg ttcacagttg tattccagta |
| 3901 | ttcaagatag attcctgatt tttcaattag gaaaagtaaa atccaaaatg ttagcaaaac |
| 3961 | aaagtgcaat attaaatgtt tgctttatag attatattct atggctgttt gtaatttctc |
| 4021 | tttttttcct tttttatttg gtgctgaata tgtccttgta ggctctgttt taagaaaaca |
| 4081 | atatgtggga aatgatttaa tttttcctat tgctcttcct tgtggaaaat aaagtgtttt |
| 4141 | gtttttttct gttttgtata aaaaaaaaaa aaaaaaaa |

In some embodiments, a modulator for use in the methods, compositions and kits disclosed herein is a gene silencing RNAi of the CaMKK2 β2-AR regulator gene which is a siRNA. In some embodiments, one can use any gene silencing siRNA which targets a region of the sequence of human CaMKK2 gene, which as the following sequence: NM_{—}172226.2. To avoid doubt, the sequence of a human CaMKK2 cDNA is provided at, for example, GenBank Accession Nos. NM_{—}172226.2 can be used for the production of siRNA. The sequence of human CaMKK2 is the following sequence:

| (SEQ ID NO: 30) | |
|---|---|
| 1 | gcgcgcccgc cgcccgggcg gaggagagga gcgcgcggcc gcgcagagca agctgagccg |
| 61 | agccgagccg agctgggggc gcagagcgcg ggaggcggcg gcggcgcgga gcccaggtgg |
| 121 | ctccgctgcc ggatgggagt gccccagtgt gctggatgaa gctggcgcat gcaccatgtc |
| 181 | atcatgtgtc tctagccagc ccagcagcaa ccgggccgcc ccccaggatg agctgggggg |
| 241 | caggggcagc agcagcagcg aaagccagaa gccctgtgag gccctgcggg gcctctcatc |
| 301 | cttgagcatc cacctgggca tggagtcctt cattgtggtc accgagtgtg agccgggctg |
| 361 | tgctgtggac ctcggcttgg cgcgggaccg gcccctggag gccgatggcc aagaggtccc |
| 421 | ccttgacacc tccgggtccc aggcccggcc ccacctctcc ggtcgcaagc tgtctctgca |
| 481 | agagcggtcc cagggtgggc tggcagccgg tggcagcctg gacatgaacg gacgctgcat |
| 541 | ctgcccgtcc ctgccctact cacccgtcag ctccccgcag tcctcgcctc ggctgccccg |
| 601 | gcggccgaca gtggagtctc accacgtctc catcacgggt atgcaggact gtgtgcagct |
| 661 | gaatcagtat accctgaagg atgaaattgg aaagggctcc tatggtgtcg tcaagttggc |
| 721 | ctacaatgaa aatgacaata cctactatgc aatgaaggtg ctgtccaaaa agaagctgat |
| 781 | ccggcaggcc ggctttccac gtcgccctcc accccgaggc acccggccag ctcctggagg |
| 841 | ctgcatccag cccaggggcc ccattgagca ggtgtaccag gaaattgcca tcctcaagaa |
| 901 | gctggaccac cccaatgtgg tgaagctggt ggaggtcctg gatgacccca atgaggacca |
| 961 | tctgtacatg gtgttcgaac tggtcaacca agggcccgtg atggaagtgc ccaccctcaa |
| 1021 | accactctct gaagaccagg cccgtttcta cttccaggat ctgatcaaag gcatcgagta |
| 1081 | cttacactac cagaagatca tccaccgtga catcaaacct tccaacctcc tggtcggaga |
| 1141 | agatgggcac atcaagatcg ctgactttgg tgtgagcaat gaattcaagg gcagtgacgc |
| 1201 | gctcctctcc aacaccgtgg gcacgcccgc cttcatggca cccgagtcgc tctctgagac |
| 1261 | ccgcaagatc ttctctggga aggccttgga tgtttgggcc atgggtgtga cactatactg |
| 1321 | ctttgtcttt ggccagtgcc cattcatgga cgagcggatc atgtgtttac acagtaagat |
| 1381 | caagagtcag gccctggaat ttccagacca gcccgacata gctgaggact tgaaggacct |
| 1441 | gatcacccgt atgctggaca agaaccccga gtcgaggatc gtggtgccgg aaatcaagct |
| 1501 | gcacccctgg gtcacgaggc atggggcgga gccgttgccg tcggaggatg agaactgcac |
| 1561 | gctggtcgaa gtgactgaag aggaggtcga gaactcagtc aaacacattc ccagcttggc |
| 1621 | aaccgtgatc ctggtgaaga ccatgatacg taaacgctcc tttgggaacc cattcgaggg |
| 1681 | cagccggcgg gaggaacgct cactgtcagc gcctggaaac ttgctcacga agcaaggcag |
| 1741 | cgaagacaac ctccagggca ccgacccgcc ccccgtgggg gaggaggaag tgctcttgtg |
| 1801 | agaggcagtc cctgcgtgga aagttgctgg gcccccgccc ccggctcccc cgcacgcatg |
| 1861 | catccactgc ggccggagga ggccatggag cccgagtagc tgcctggatc gctcgacctc |
| 1921 | gcatgcgcgc cgcgtcgcct ctggggggct gctgcaccgc gtttccatag cagcatgtcc |
| 1981 | tacggaaacc cagcacgtgt gtagagcctc gatcgtcatc tctggttatt tgttttttcc |
| 2041 | tttgttgttt taaaggggac aaaaaaaaaa aaaggacttg actccatgac gtcgaccgtg |
| 2101 | gccgctggct ggctggacag gcgggtgtga ggagttgcag acccaaaccc acgtgcattt |
| 2161 | tgggacaatt gctttttaaa acgtttttat gccaaaaatc cttcattgtg attttcagaa |
| 2221 | ccacgtcaga tataccaagt gactgtgtgt ggggtttgac aactgtggaa aggcgagcag |
| 2281 | aaaactccgg cggtctgagg ccatggaggt ggttgctgca tttgagaggg agtagggggc |
| 2341 | tagatgtggc tcctagtgca aaccggaaac catggcacct tccagagccg tggtctcaag |
| 2401 | gagtcagagc agggctggcc ctcagtagct gcagggagct ttgatgcaac ttatttgtaa |
| 2461 | gaaggatttt taaatttttt atgggtagaa ttgtagtcag gaaaacagaa agggcttgaa |
| 2521 | atttaataag tgctgctgga aggggatttt ccaagcctgg aagggtattc agcagctgtg |
| 2581 | gtggggaaac atttctcctg aaagactgaa cgtgtttctt catgacagct gctcaaagca |
| 2641 | ggtttctgag atagctgacc gagctctggt aaatctcttt gtcaaattac gaaaacttca |
| 2701 | gggtgaaatc ctatgcttcc atgtacatta catggcttaa gattaaacaa aaacattttt |
| 2761 | caagtctcta actagagtga actctagagc acagtagttc agaaactatt tagagcttcc |
| 2821 | aggatatatt tcacagcttc aggcatgtga tcagttagag ccgatgaaac ctatgcccgc |
| 2881 | ctgtatatat attagcagct tagctagttc ataacctgta tattctaaag actgctaagg |
| 2941 | ttttgttttc attttaaatc ctagctgatt gttgtggtca atgaaatacc cagtttctgg |
| 3001 | agggccaggt gggaaatgct ttcactggac caacacacaa atgatcatcc tgaggatctg |
| 3061 | agcttcccta gactccacac aataaccttg gggcaccctt ttagagaaga ctgttgaaac |
| 3121 | ccacagcact cgttggggta tgaggaaacc agggcttggc acaggaagtt cccctttgta |
| 3181 | gctaaaagtc cagaaagaaa gggttcatct ttttgacttc caactgatat tgggaagttt |
| 3241 | ggttgaggtt caagtgtgac tccttccaga gccacaggta ggggagtgtg aagttgaggg |
| 3301 | ggaggaaagc tggaaggact ctgccttggg agattcccag ctctgctttc cagcgcttgg |
| 3361 | tggaatctgg gctggggaaa gacggcaccg ggaaactctg cttccccatt gtttccatct |
| 3421 | gatcagctgt ggtgtgagga cttctcagac aaaggcaagg cctcgtgccc ctgcccagcc |
| 3481 | cattcatgga gccctgggcc ttcttggctt ccatagatcc taagctcttg actgtagttt |
| 3541 | agccagactt gttttgctat cttataagca gttcagaatt agggaatgct ggttttgaag |
| 3601 | agcaaaggac aggtagtcta gagagggtcg tctggcctgc ttgctgggtc tttgtaaccc |
| 3661 | agcacttcct cttgccctcc tggctttatg tttatgggga gaggactcaa tagctccacc |
| 3721 | ccttctggca ccagatgggg cttggttagt ttgcaataag caccttgcag aggttaaagc |
| 3781 | cagcgggtcc ctagtcttag gcccagcctg cttgtgtggg ctctggcctg gcctggtggc |
| 3841 | tggcccaggg ggcagcagtg cttagagctt ctgcagggct tctcttgttt acacagctgc |
| 3901 | atcagacaat gccatttctc cccaccacgg aaccttccat ctaagatttc ttccagggaa |
| 3961 | tgccagcaat caggcagcac ccagctgtgg gggcagtggg gtgggggaga cccacattga |
| 4021 | tgactttttt tttttctttt aatgaagaaa caccaaagaa agctgtggaa aggacctgcc |
| 4081 | ccacatgaaa aggataagcc aagatggctg taaacacaga gcatttgagc tgccactctt |
| 4141 | ggagcacatt gatttttcaa aagccagctc tgtcaggaaa ggaggtgctg ttatgagcag |
| 4201 | ctcttccagt gggcaaagag gacgcccata atttcttcca ttgctagctc atctgtggga |
| 4261 | ccaatttggt gtaagcaacc tgtggcctgc acttgtggcc tcgaaggaag cacaaaccct |
| 4321 | ccatccactt cccatttcct ctgccctttt ccacctcccc cttccatccc accagctgcc |
| 4381 | agtggctccc agaaagcctt attgagcccc ttgttgacac ttggggctgc ggaggcctct |
| 4441 | ccctactggt ctggcctttc ctgagaggca ggtcttccgt cctcagagcc tttctggaac |
| 4501 | aaggagaatg cctgtgcagg tggacacaca ggcctggcct gtcgctctca cttgtcttcc |
| 4561 | agcggggagc ttcacgttgc cgagtggaag aaccatgacc tccacttgct tccaaggtgc |
| 4621 | tagggaagtt tcagggtacg ctggttcccc tctccagctg gaggccgagt ttctggggac |
| 4681 | tgcagatttt tctactctgt gatcgattca atgcccgatg cttctgtttc attcccgacc |
| 4741 | ctttctacta tgcattttcc ttttatcagg tgtataaagt taaatactgt gtatttatca |
| 4801 | ctaaaaagta catgaactta agagacaact aagcctttcg tgtttttcca caggtgttta |
| 4861 | agcttctctg tacagttgaa ataaacagac agcaaaatgg tgccaaaaaa aaaaaaaaaa |
| 4921 | aaa |

In some embodiments, a modulator for use in the methods, compositions and kits disclosed herein is a gene silencing RNAi of the AAEST β2-AR regulator gene which is a siRNA. In some embodiments, one can use any gene silencing siRNA which targets a region of the sequence of human AAEST transcript, which has the sequence corresponding to SEQ ID NO: 36 as disclosed herein.

In some embodiments, a siRNA agent is a gene silencing RNAi agent, e.g. for decreasing the expression of β2-AR regulator gene mRNA such as FDPS mRNA and/or ARRDC3 and/or CaMKK2 mRNA. In alternative embodiments, a siRNA agent is a gene activating agent, e.g. for increasing (or upregulating) the expression of β2-AR regulator gene mRNA such as latrophilin 2 mRNA.

In some embodiments, siRNA sequences are chosen to maximize the uptake of the antisense (guide) strand of the siRNA into RISC and thereby maximize the ability of RISC to target human β2-AR regulator gene, e.g. FDPS, ARRDC3, CaMKK2, AAEST and latrophilin 2, mRNA. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC.

In a preferred embodiment, the siRNA or modified siRNA, such as gene silencing RNAi agents, and/or gene activating RNAi agents are delivered in a pharmaceutically acceptable carrier. Additional carrier agents, such as liposomes, can be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into siRNA capable of targeting, for example, β2-AR regulator gene, e.g. FDPS, ARRDC3, CaMKK2, AAEST and latrophilin 2. In one embodiment, the vector can be a regulatable vector, such as tetracycline inducible vector.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents, e.g., the siRNAs used in the methods of the invention.

Other strategies for delivery of the RNA interfering agents, e.g., the siRNAs or shRNAs used in the methods of the invention, can also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

As noted, the dsRNA, such as siRNA or shRNA can be delivered using an inducible vector, such as a tetracycline inducible vector. Methods described, for example, in Wang et al. Proc. Natl. Acad. Sci. 100: 5103-5106, using pTet-On vectors (BD Biosciences Clontech, Palo Alto, Calif.) can be used. In some embodiments, a vector can be a plasmid vector, a viral vector, or any other suitable vehicle adapted for the insertion and foreign sequence and for the introduction into eukaryotic cells. The vector can be an expression vector capable of directing the transcription of the DNA sequence of the agonist or antagonist nucleic acid molecules into RNA. Viral expression vectors can be selected from a group comprising, for example, reteroviruses, lentiviruses, Epstein Barr virus-, bovine papilloma virus, adenovirus- and adeno-associated-based vectors or hybrid virus of any of the above. In one embodiment, the vector is episomal. The use of a suitable episomal vector provides a means of maintaining the antagonist nucleic acid molecule in the subject in high copy number extra chromosomal DNA thereby eliminating potential effects of chromosomal integration.

RNA interference molecules and nucleic acid inhibitors useful in the methods as disclosed herein can be produced using any known techniques such as direct chemical synthesis, through processing of longer double stranded RNAs by exposure to recombinant Dicer protein or Drosophila embryo lysates, through an in vitro system derived from S2 cells, using phage RNA polymerase, RNA-dependant RNA polymerase, and DNA based vectors. Use of cell lysates or in vitro processing can further involve the subsequent isolation of the short, for example, about 21-23 nucleotide, siRNAs from the lysate, etc. Chemical synthesis usually proceeds by making two single stranded RNA-oligomers followed by the annealing of the two single stranded oligomers into a double stranded RNA. Other examples include methods disclosed in WO 99/32619 and WO 01/68836 that teach chemical and enzymatic synthesis of siRNA. Moreover, numerous commercial services are available for designing and manufacturing specific siRNAs (see, e.g., QIAGEN Inc., Valencia, Calif. and AMBION Inc., Austin, Tex.)

In some embodiments, an agent is protein or polypeptide or RNAi agent modulates the expression of a β2-AR regulator gene, e.g. FDPS or ARRDC3 or CaMKK2 or AAEST or latrophilin 2. In such embodiments cells can be modified (e.g., by homologous recombination) to provide increased expression of such an agent, for example by replacing, in whole or in part, the naturally occurring promoter with all or part of a heterologous promoter so that the cells express the modulator of a β2-AR regulator gene, e.g. FDPS, ARRDC3, CaMKK2, AAEST and latrophilin 2, for example a protein or RNAi agent (e.g. gene silencing- or gene activating-RNAi agent). Typically, a heterologous promoter is inserted in such a manner that it is operatively linked to the desired nucleic acid encoding the agent. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also can be engineered to express an endogenous gene comprising the inhibitor agent under the control of inducible regulatory elements, in which case the regulatory sequences of the endogenous gene can be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al. The agent can be prepared by culturing transformed host cells under culture conditions suitable to express the miRNA. The resulting expressed agent can then be purified from such culture (i.e., from culture medium or cell extracts) using known purification processes, such as gel filtration and ion exchange chromatography. The purification of the peptide or nucleic acid agent inhibitor of a β2-AR regulator gene can also include an affinity column containing agents which will bind to the protein; one or more column steps over such affinity resins as concanavalin A-agarose, Heparin-toyopearl™ or Cibacrom blue 3GA Sepharose; one or more steps involving hydrophobic interaction chromatography using such resins as phenyl ether, butyl ether, or propyl ether; immunoaffnity chromatography, or complementary cDNA affinity chromatography.

In one embodiment, a nucleic acid modulator of a β2-AR regulator gene, e.g. (gene silencing- or gene activating RNAi agent) can be obtained synthetically, for example, by chemically synthesizing a nucleic acid by any method of synthesis known to the skilled artisan. A synthesized nucleic acid modulator of a β2-AR regulator gene, e.g. FDPS, ARRDC3, CaMKK2, AAEST and latrophilin 2 can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

In some circumstances, for example, where increased nuclease stability of a nucleic acid inhibitor is desired, nucleic acids having nucleic acid analogs and/or modified internucleoside linkages can be used. Nucleic acids containing modified internucleoside linkages can also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), diinethylene-sulfoxide (—CH₂—SO—CH₂), dimethylene-sulfone (—CH₂—SO₂-CHD, 2′-O-alkyl, and 2′-deoxy-2′-fluoro ‘phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., 5,714,606 to Acevedo, et al, 5,378,825 to Cook, et al., 5,672,697 and 5,466,786 to Buhr, et al., 5, 777,092 to Cook, et al., 5,602,240 to De Mesmacker, et al., 5,610,289 to Cook, et al. and 5,858,988 to Wang, also describe nucleic acid analogs for enhanced nuclease stability and cellular uptake.

Synthetic siRNA molecules, including shRNA molecules, can also easily be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but are not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell. 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

In some embodiments, the where a modulator is a gene silencing siRNA molecule which targets a specific β2-AR regulator gene, the siRNA targets the coding mRNA sequence of FDPS, ARRDC3, beginning from about 25 to 50 nucleotides, from about 50 to 75 nucleotides, or from about 75 to 100 nucleotides downstream of the start codon. One method of designing a siRNA molecule of the present invention involves identifying the 29 nucleotide sequence motif AA(N29)TT (where N can be any nucleotide), and selecting hits with at least 25%, 30%, 35%, 40%, 45%, 50%, 55%, 60%, 65%, 70% or 75% G/C content. The “TT” portion of the sequence is optional. Alternatively, if no such sequence is found, the search can be extended using the motif NA(N21), where N can be any nucleotide. In this situation, the 3′ end of the sense siRNA can be converted to TT to allow for the generation of a symmetric duplex with respect to the sequence composition of the sense and antisense 3′ overhangs. The antisense siRNA molecule can then be synthesized as the complement to nucleotide positions 1 to 21 of the 23 nucleotide sequence motif. The use of symmetric 3′ TT overhangs can be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but are not limited to the NCBI, BLAST, Derwent and GenSeq as well as commercially available oligosynthesis software such as Oligoengine®, can also be used to select siRNA sequences against EST libraries to ensure that only one gene is targeted.

In some embodiments, where the modulator is a gene activating RNAi agent, e.g. for upregulating latrophilin 2 expression or protein levels, a RNAi modulator agent can target nucleotide sequences can contain 5′ or 3′ UTRs and regions nearby the start codon.

Delivery of RNA Interfering Agents:

Methods of delivering RNAi agents, e.g., an siRNA, or vectors containing an RNAi agent, to the target cells (e.g., basal cells or cells of the lung ad/or respiratory system or other desired target cells) are well known to persons of ordinary skill in the art. In some embodiments, a RNAi agent (e.g. gene silencing- or gene activating RNAi agent) which targets a β2-AR regulator gene, e.g. FDPS, ARRDC3, CaMKK2, AAEST and latrophilin 2 can be administered to a subject via aerosol means, for example using a nebulizer and the like. In alternative embodiments, administration of a RNAi agent (e.g. gene silencing- or gene activating RNAi agent) which targets a β2-AR regulator gene, e.g. FDPS, ARRDC3, CaMKK2, AAEST and latrophilin 2 can include, for example (i) injection of a composition containing the RNA interfering agent, e.g., an siRNA, or (ii) directly contacting the cell, e.g., a cell of the respiratory system, with a composition comprising an RNAi agent, e.g., an siRNA. In another embodiment, RNAi agents, e.g., an siRNA can be injected directly into any blood vessel, such as vein, artery, venule or arteriole, via, e.g., hydrodynamic injection or catheterization. In some embodiments an RNAi (e.g. gene silencing- or gene activating RNAi agent) targeting a β2-AR regulator gene, e.g. FDPS, ARRDC3, CaMKK2, AAEST and latrophilin 2 can delivered to specific organs, for example the liver, bone marrow or systemic administration.

Administration can be by a single injection or by two or more injections. In some embodiments, a RNAi agent is delivered in a pharmaceutically acceptable carrier. One or more RNAi agents can be used simultaneously, e.g. one or more gene silencing RNAi agents which inhibit one or more, in any combination, of the β2-AR regulator genes, e.g. FDPS or ARRDC3 or CaMKK2 or AAEST, can be used with one or more gene activating RNAi agents which target a β2-AR regulator gene such as latrophilin 2 or KIAA0786. The RNA interfering agents, e.g., the siRNAs targeting a β2-AR regulator gene, e.g. FDPS, ARRDC3, CaMKK2, AAEST and latrophilin 2, can be delivered singly, or in combination with other RNA interfering agents, e.g., siRNAs, such as, for example siRNAs directed to other cellular genes. A gene silencing- and/or a gene activating RNAi agent which targets a β2-AR regulator gene, e.g. FDPS, ARRDC3, CaMKK2, AAEST and latrophilin 2 can also be administered in combination with other pharmaceutical agents which are used to treat or prevent neurodegenerative diseases or disorders.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNAi effectively into cells. For example, an antibody-protamine fusion protein when mixed with an siRNA, binds siRNA and selectively delivers the siRNA into cells expressing an antigen recognized by the antibody, resulting in silencing of gene expression only in those cells that express the antigen which is identified by the antibody. In some embodiments, the antibody can be any antibody which identifies an antigen expressed on cells expressing β2-AR. In some embodiments, the antibody is an antibody which binds to an β2-AR antigen, but where the antibody does not inhibit β2-AR function. In some embodiments, the siRNA can be conjugated to a β2-AR agonist, for example where the β2-AR agonist is a polypeptide, and where the conjugation with the RNAi does not interrupt the function of the β2-AR agonist.

In some embodiments, a siRNA or RNAi binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein.

In some embodiments, a viral-mediated delivery mechanism can also be employed to deliver siRNAs, e.g. siRNAs (e.g. gene silencing- or gene activating RNAi agents) which target a β2-AR regulator gene, e.g. FDPS, ARRDC3 and latrophilin 2, to cells in vitro and in vivo as described in Xia, H. et al. (2002) Nat Biotechnol 20(10):1006). Plasmid- or viral-mediated delivery mechanisms of shRNA can also be employed to deliver shRNAs to cells in vitro and in vivo as described in Rubinson, D. A., et al. ((2003) Nat. Genet. 33:401-406) and Stewart, S. A., et al. ((2003) RNA 9:493-501). Alternatively, in other embodiments, a RNAi agent, e.g., a gene silencing- or gene activating RNAi agent which targets a β2-AR regulator gene, e.g. FDPS, ARRDC3 and latrophilin 2, can also be introduced into cells via the vascular or extravascular circulation, the blood or lymph system, and the cerebrospinal fluid.

The dose of the particular RNAi agent will be in an amount necessary to effect RNA interference, e.g., gene silencing of the particular β2-AR regulator gene, e.g. FDPS or ARRDC3, or CaMKK2, or AAEST or gene activating of a particular β2-AR regulator gene such as latrophilin 2 thereby leading to modification (e.g. inhibit or increase) the target β2-AR regulator gene expression level and subsequent decrease or increase in the protein level encoded by the target gene.

It is also known that RNAi molecules do not have to match perfectly to their target sequence. Preferably, however, the 5′ and middle part of the antisense (guide) strand of the siRNA is perfectly complementary to the target nucleic acid sequence of a β2-AR regulator gene, e.g. FDPS or ARRDC3 CaMKK2 or AAEST or a region of the 5′ upstream target sequence of latrophilin 2 (e.g. AA496068 (SEQ ID NO: 5)).

Accordingly, the RNAi molecules functioning as gene silencing- or gene activating RNAi agents of a β2-AR regulator gene, e.g. FDPS, ARRDC3, CaMKK2, AAEST and latrophilin 2 as disclosed herein are for example, but are not limited to, unmodified and modified double stranded (ds) RNA molecules including short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also can contain 3′ overhangs, preferably 3′UU or 3′TT overhangs. In one embodiment, the siRNA molecules of the present invention do not include RNA molecules that comprise ssRNA greater than about 30-40 bases, about 40-50 bases, about 50 bases or more. In one embodiment, the siRNA molecules of the present invention are double stranded for more than about 25%, more than about 50%, more than about 60%, more than about 70%, more than about 80%, more than about 90% of their length.

In some embodiments, a gene silencing RNAi nucleic acid inhibitor of a β2-AR regulator gene, e.g. FDPS, ARRDC3, CaMKK2, AAEST is any agent which binds to and inhibits the expression of a β2-AR regulator gene, e.g. FDPS or, ARRDC3 or CaMKK2 or AAEST mRNA, where the expression of the β2-AR regulator gene, e.g. FDPS, ARRDC3 or CaMKK2 is inhibited or in the case of AAEST, inhibition of the expression of the AAEST miRNA.

In another embodiment of the invention, agents inhibiting a β2-AR regulator gene, e.g. FDPS, ARRDC3, CaMKK2 are catalytic nucleic acid constructs, such as, for example ribozymes, which are capable of cleaving RNA transcripts and thereby preventing the production of wildtype protein. Ribozymes are targeted to and anneal with a particular sequence by virtue of two regions of sequence complementary to the target flanking the ribozyme catalytic site. After binding, the ribozyme cleaves the target in a site specific manner. The design and testing of ribozymes which specifically recognize and cleave sequences of the gene products described herein, for example for cleavage of a β2-AR regulator gene, e.g. FDPS or ARRDC3 or CaMKK2 or AAEST can be achieved by techniques well known to those skilled in the art (for example Lleber and Strauss, (1995) Mol Cell Biol 15:540.551, the disclosure of which is incorporated herein by reference).

Proteins and Peptide Inhibitors of a β2-AR Regulator Gene.

In some embodiments, modulator that inhibits a β2-AR regulator gene, e.g. FDPS or ARRDC3 or CaMKK2 or AAEST is a protein and/or peptide inhibitor of the β2-AR regulator gene, e.g. FDPS, ARRDC3, CaMKK2 or AAEST for example, but are not limited to mutated proteins; therapeutic proteins and recombinant proteins. Proteins and peptides inhibitors can also include for example mutated proteins, genetically modified proteins, peptides, synthetic peptides, recombinant proteins, chimeric proteins, antibodies, humanized proteins, humanized antibodies, chimeric antibodies, modified proteins and fragments thereof.

In some embodiments, an agent inhibitor of a β2-AR regulator gene, e.g. FDPS or ARRDC3 or CaMKK2 or AAEST is a dominant negative variants of the β2-AR regulator gene, for example a non-functional variant of the β2-AR regulator gene, e.g. FDPS or ARRDC3 or CaMKK2 or AAEST.

Antibodies

In some embodiments, modulators which are inhibitors of genes and/or gene products useful in the methods of the present invention include, for example, antibodies, including monoclonal, chimeric humanized, and recombinant antibodies and antigen-binding fragments thereof. In some embodiments, neutralizing antibodies can be used as inhibitors of the protein expressed by a β2-AR regulator gene, e.g. FDPS and/or ARRDC3 and/or CaMKK2 and/or AAEST β2-AR regulator gene. Antibodies are readily raised in animals such as rabbits or mice by immunization with the antigen. Immunized mice are particularly useful for providing sources of B cells for the manufacture of hybridomas, which in turn are cultured to produce large quantities of monoclonal antibodies.

In one embodiment of this invention, the inhibitor to the gene products identified herein can be an antibody molecule or the epitope-binding moiety of an antibody molecule and the like. Antibodies provide high binding avidity and unique specificity to a wide range of target antigens and haptens. Monoclonal antibodies useful in the practice of the present invention include whole antibody and fragments thereof and are generated in accordance with conventional techniques, such as hybridoma synthesis, recombinant DNA techniques and protein synthesis.

Useful monoclonal antibodies and fragments can be derived from any species (including humans) or can be formed as chimeric proteins which employ sequences from more than one species. Human monoclonal antibodies or “humanized” murine antibody are also used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction the possibly of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is the smallest antibody fragment developed to date. Techniques have been developed to display scFv molecules on the surface of filamentous phage that contain the gene for the scFv. scFv molecules with a broad range of antigenic-specificities can be present in a single large pool of scFv-phage library. Some examples of high affinity monoclonal antibodies and chimeric derivatives thereof, useful in the methods of the present invention, are described in the European Patent Application EP 186,833; PCT Patent Application WO 92/16553; and U.S. Pat. No. 6,090,923.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

One limitation of scFv molecules is their monovalent interaction with target antigen. One of the easiest methods of improving the binding of a scFv to its target antigen is to increase its functional affinity through the creation of a multimer. Association of identical scFv molecules to form diabodies, triabodies and tetrabodies can comprise a number of identical Fv modules. These reagents are therefore multivalent, but monospecific. The association of two different scFv molecules, each comprising a VH and VL domain derived from different parent Ig will form a fully functional bispecific diabody. A unique application of bispecific scFvs is to bind two sites simultaneously on the same target molecule via two (adjacent) surface epitopes. These reagents gain a significant avidity advantage over a single scFv or Fab fragments. A number of multivalent scFv-based structures has been engineered, including for example, miniantibodies, dimeric miniantibodies, minibodies, (scFv)₂, diabodies and triabodies. These molecules span a range of valence (two to four binding sites), size (50 to 120 kDa), flexibility and ease of production. Single chain Fv antibody fragments (scFvs) are predominantly monomeric when the VH and VL domains are joined by, polypeptide linkers of at least 12 residues. The monomer scFv is thermodynamically stable with linkers of 12 and 25 amino acids length under all conditions. The noncovalent diabody and triabody molecules are easy to engineer and are produced by shortening the peptide linker that connects the variable heavy and variable light chains of a single scFv molecule. The scFv dimers are joined by amphipathic helices that offer a high degree of flexibility and the miniantibody structure can be modified to create a dimeric bispecific (DiBi) miniantibody that contains two miniantibodies (four scFv molecules) connected via a double helix. Gene-fused or disulfide bonded scFv dimers provide an intermediate degree of flexibility and are generated by straightforward cloning techniques adding a C-terminal Gly4Cys (SEQ ID NO: 31) sequence. scFv-CH3 minibodies are comprised of two scFv molecules joined to an IgG CH3 domain either directly (LD minibody) or via a very flexible hinge region (Flex minibody). With a molecular weight of approximately 80 kDa, these divalent constructs are capable of significant binding to antigens. The Flex minibody exhibits impressive tumor localization in mice. Bi- and tri-specific multimers can be formed by association of different scFv molecules. Increase in functional affinity can be reached when Fab or single chain Fv antibody fragments (scFv) fragments are complexed into dimers, trimers or larger aggregates. The most important advantage of multivalent scFvs over monovalent scFv and Fab fragments is the gain in functional binding affinity (avidity) to target antigens. High avidity requires that scFv multimers are capable of binding simultaneously to separate target antigens. The gain in functional affinity for scFv diabodies compared to scFv monomers is significant and is seen primarily in reduced off-rates, which result from multiple binding to two or more target antigens and to rebinding when one Fv dissociates. When such scFv molecules associate into multimers, they can be designed with either high avidity to a single target antigen or with multiple specificities to different target antigens. Multiple binding to antigens is dependent on correct alignment and orientation in the Fv modules. For full avidity in multivalent scFvs target, the antigen binding sites must point towards the same direction. If multiple binding is not sterically possible then apparent gains in functional affinity are likely to be due the effect of increased rebinding, which is dependent on diffusion rates and antigen concentration. Antibodies conjugated with moieties that improve their properties are also contemplated for the instant invention. For example, antibody conjugates with PEG that increases their half-life in vivo can be used for the present invention. Immune libraries are prepared by subjecting the genes encoding variable antibody fragments from the B lymphocytes of naive or immunized animals or patients to PCR amplification. Combinations of oligonucleotides which are specific for immunoglobulin genes or for the immunoglobulin gene families are used. Immunoglobulin germ line genes can be used to prepare semisynthetic antibody repertoires, with the complementarity-determining region of the variable fragments being amplified by PCR using degenerate primers. These single-pot libraries have the advantage that antibody fragments against a large number of antigens can be isolated from one single library. The phage-display technique can be used to increase the affinity of antibody fragments, with new libraries being prepared from already existing antibody fragments by random, codon-based or site-directed mutagenesis, by shuffling the chains of individual domains with those of fragments from naive repertoires or by using bacterial mutator strains.

Alternatively, a SCID-hu mouse, for example the model developed by Genpharm, can be used to produce antibodies, or fragments thereof. In one embodiment, a new type of high avidity binding molecule, termed peptabody, created by harnessing the effect of multivalent interaction is contemplated. A short peptide ligand was fused via a semirigid hinge region with the coiled-coil assembly domain of the cartilage oligomeric matrix protein, resulting in a pentameric multivalent binding molecule. In preferred embodiment of this invention, ligands and/or chimeric inhibitors can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. Alternatively, two or more active agents and or inhibitors attached to targeting moieties can be administered, wherein each conjugate includes a targeting moiety, for example, a different antibody. Each antibody is reactive with a different target site epitope (associated with the same or a different target site antigen). The different antibodies with the agents attached accumulate additively at the desired target site. Antibody-based or non-antibody-based targeting moieties can be employed to deliver a ligand or the inhibitor to a target site. Preferably, a natural binding agent for an unregulated or disease associated antigen is used for this purpose.

### β2-AR Agonists

Examples of the β2-AR agonists which can be used in compositions (e.g. admixtures) with an inhibitor of β2-AR regulator gene can be any known β2-AR agonist, for example, but not limited to Salbutamol (referred to also as Albuterol in USA), Bitolterol mesylate, Formoterol, Isoprenaline, Levalbuterol, Metaproterenol, Salmeterol, Terbutaline, Ritodrine.

In some embodiments, a β2-AR agonist in a composition also comprising an inhibitor of a β2-AR regulator gene is useful in the treatment and prevention of diseases mediated by β2 adrenergic receptor such as asthma, bronchitis, and the like. β2-AR agonists are also useful in the treatment of nervous system injury and premature labor. It is also contemplated that the compounds of this invention are useful for treating metabolic disorders such as obesity, diabetes, and the like.

In some embodiments, the effect of the modulator of a β2-AR regulator gene on the agonist activity of a β2-AR agonist can be determined using variety of in vitro assays known to those of ordinary skill in the art, such as the assay described in the Examples 2-4. In some embodiments, such assays can be used to determine the ability of a modulator of a β2-AR regulator gene to enhance or increase the agonist activity of a β2-AR agonist. The ability of a modulator of a β2-AR regulator gene to enhance or increase the agonist activity of a β2-AR may also be assayed using the assays as disclosed in U.S. Pat. No. 6,683,611, which is incorporated herein by reference, or by the ex vivo assays described in Ball, D. I. et al., “Salmterol a Novel, Long-acting beta 2-Adrenergic Agonist: Characterization of Pharmacological Activity in Vitro and in Vivo” Br. J. Pharmocol., 104, 665-671 (1991); Linden, A. et al., “Sameterol, Formoterol, and Salbutamol in the Isolated Guinea-Pig Trachea: Differences in Maximum Relaxant Effect and Potency but not in Functional Atagonism, Thorax, 48, 547-553, (1993); and Bials, A. T. et al., Inventigations into Factors Determining the Duration of Action of the Beta 2-Adrenoceptor Agonist, Salmateroal. Br. J. Pharmacol., 108, 505-515 (1993); or in vivo assays such as those described in Ball, D. I. et al., “Salmterol a Novel, Long-acting beta 2-Adrenergic Agonist: Characterization of Pharmacological Activity in Vitro and in Vivo” Br. J. Pharmacol., 104, 665-671 (1991); Kikkawa, H. et al., “RA-2005, a Novel, Long-acting, and Selective Beta 2-Adrenoceptor Agonist: Characterization of its in vivo Bronchodilating Action in Guinea Pigs and Cats in Comparison with other Beta 2-Agonists”, Biol. Pharm. Bull., 17, 1047-1052, (1994); and Anderson, G. P., “Formeterol: Pharmacology, Colecular basis of Agonism and Mechanism of Long Duration of a Highly Potent and Selective Beta 2-Adrenoceptor Agonist Bronchodilator, Life Sciences, 52, 2145-2160, (1993).

### Pharmaceutical Compositions, Combinations and Admixtures

In some embodiments, a pharmaceutical composition comprises at least one β2-AR agonist and at least one modulator of a β2-AR regulator gene, and optionally a pharmaceutically acceptable carrier. In some embodiments, the pharmaceutical composition comprises at least about 20% lower dose of the daily recommended β2-AR agonist, where the modulator of the β2-AR regulator prolongs the duration of the β2-AR agonist by at least 20%. In some embodiments, the pharmaceutical composition comprises at least about 20%, or at least about 30% or at least about 40% or at least about 50% or at least about 60% or greater than a 60% lower dose of the daily recommended β2-AR agonist.

The compositions encompassed by the invention may further comprise at least one pharmaceutically acceptable excipient. Excepients useful for preparing the dosages forms from the composition according to the invention and the instruments necessary to prepare them are described in U.S. Publication No.: 2003/0206954 and 2004/0052843.

One aspect of the invention relates to modulators of β2-AR regulator genes, e.g. FDPS, ARRDC3, CaMKK2, AAEST Latrophilin 2. The modification (e.g. increase or decreased activity) of a β2-AR regulator gene, e.g. FDPS, ARRDC3, CaMKK2, AAEST by inhibition (or activation of latrophilin 2) can in some embodiments potentiate the effectiveness of β2-AR agonist, for example if the amount of β2-AR agonist used in combination with a modulator of β2-AR regulator genes, e.g. FDPS, ARRDC3, CaMKK2, AAEST, Latrophilin 2, the amount of β2-AR agonist can be reduced by at least 10% without adversely affecting the result, for example, without adversely effecting the level of β2-AR activation. In another embodiment, the criteria used to select a modulator of a β2-AR regulator gene, (e.g. an inhibitor of FDPS, ARRDC3, CaMKK2, AAEST or an activator of Latrophilin 2 or KIAA0786) is a reduction of at least . . . 10%, . . . 15%, . . . 20%, . . . 25%, . . . 35%, . . . 50%, . . . 60%, . . . 90% and all amounts in-between of the amount of β2-AR agonist to be used without adversely effecting the β2-AR agonist function when compared to the similar amount of β2-AR agonist in cell without the addition of modulator of the β2-AR regulator gene.

For administration to a subject, a modulator of a β2-AR regulator gene, e.g. an inhibitor of FDPS or ARRDC3 or CaMKK2 or AAEST or activator latrophilin 2, alone or in combination with a β2-AR agonist, can be provided in pharmaceutically acceptable compositions. A pharmaceutically acceptable composition can comprise a therapeutically-effective amount of a modulator of a β2-AR regulator gene, e.g. an inhibitor of FDPS and/or ARRDC3 and/or CaMKK2 or an activator of latrophilin 2, formulated together with one or more pharmaceutically acceptable carriers (additives) and/or diluents. In some embodiments, a formulation can comprise a modulator of a β2-AR regulator gene, e.g. an inhibitor of FDPS and/or ARRDC3 and/or CaMKK2 and/or AAEST or an activator of latrophilin 2, alone or in combination with one or more β2-AR agonists.

Combination Treatments

As disclosed herein, the modulators of the β2-AR regulator genes can be administrated to a subject alone, or optionally in combination (e.g. simultaneously with, sequentially or separately) with a pharmaceutically active agent, e.g. a β2-AR agonist as disclosed herein. In some embodiments, the composition comprising a modulator of a β2-AR regulator gene can further comprise an alternative active agents, for example, exemplary pharmaceutically active compound include, but are not limited to, those found in Harrison's principals of Internal Medicine, 13^{th} Edition, Eds. T. R. Harrison et al. McGraw-Hill N.Y., NY; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference. Without wishing to be bound by theory, administration of a modulator of a β2-AR regulator gene in combination with a β2-AR agonist can increase the effectiveness of the β2-AR agonist.

In some embodiments, the composition comprising a modulator of a β2-AR regulator gene can comprise a β2-AR agonist and further comprise an alternative active agents, for example, an asthma agent. Exemplary agents known to treat asthma include mast cell degranulation agents (i.e., Cromylyn sodium or Nedocromil sodium), leukotriene inhibitors (i.e., Monteleukast sodium, Zafirlukast, or Pranlukast hydrate), corticosteroids (i.e., Beclomethasone, Budesonide, Ciclesonide, Hydrolysable glucocorticoid, Triamcinolone acetonide, Flunisolide, Mometasone furcate, or Fluticasone propionate), IgE binding inhibitors (i.e., Omalizumab), Adenosine A2 agonists, Anti-CD23 antibody, E-Selectin antagonists, P-Selectin antagonists, L-Selectin antagonists, interlukin inhibitors/monoclonal antibodies, pulmonary surfactants, neurokinin antagonists, NF-Kappa-B inhibitors, PDE-4 inhibitors (i.e., Cilomilast, or Roflumilast), Thromboxan A2 inhibitors (i.e., Rama-go troban, or Seratrodast), tryptase inhibitors, VIP agonists or antisense agents.

In some embodiments, the pharmaceutically active agent used conjunction with the compositions as disclosed herein is an agent used to treat allergic rhinitis. Exemplary agents include, but are not limited to HI antihistamines i.e., terfendine or astemizole; alpha-adrenergic agents; and glucocorticoids, i.e., beclamethasone or flunisolide. In some embodiments, the pharmaceutically active agent is an agent to treat sinusitis, more specifically, chronic sinusitis. Exemplary agents to treat sinusitis include, but are not limited to, corticosteroids (e.g., oral, intranasal, nebulized, or inhaled); antibiotics (e.g., oral, intranasal, nebulized, inhaled or intravenous); anti-fungal agents; salt-water nasal washes and mist sprays; anti-inflammatory agents; decongestants (oral or nasal); guaifenesin; potassium iodide; leukotriene inhibitors (e.g., monteleukast); mast cell degranulating agents; topical moisterizing applications (e.g., nasal sprays or gels which may contain moisterizing agents such as propylene glycol or glycerin); hot air inhalation; mechanical devices to aid in breathing; enzymatic cleansers (e.g., papaya enzymes); and antihistame sprays.

In some embodiments, the pharmaceutically composition comprising a β2-AR agonist and a modulator of a β2-AR regulator gene is used to treat a respiratory disorder, e.g. asthma. In some embodiments, the subject is selected for having a respiratory disorder before being administered a composition comprising a β2-AR agonist and a modulator of a β2-AR regulator gene. In some embodiments, a composition comprising a β2-AR agonist and a modulator of a β2-AR regulator gene is used to treat COPD or chronic bronchitis or emphysema. In some embodiments, a subject has been screened and identified to have COPD or chronic bronchitis or emphysema prior to administration of the composition comprising a β2-AR agonist and a modulator of a β2-AR regulator gene. In some embodiments, the pharmaceutical composition comprising a β2-AR agonist and a modulator of a β2-AR regulator gene comprises additional agents to treat COPD or other respiratory disorders. Exemplary agents to treat COPD include, but are not limited to, bronchodilator drugs [e.g., short and long acting beta-2 stimulants, anticholinergics (e.g., ipratoprium bromide, theophylline compounds or a combination), steroids (topical or oral), or mucolytic agents (e.g., ambroxol, ergosterin, carbocysteine, iodinated glycerol)]; antibiotics; anti-fungals; moisterization by nebulization; anti-tussives; respiratory stimulants (e.g., doxapram, almitrine bismesylate); and alpha 1 antitrypsin administration.

In some embodiments, a composition comprising a β2-AR agonist and a modulator of a β2-AR regulator gene and pharmaceutically active agent (e.g. β2-AR agonist, and/or other active agent) can be administrated to the subject in the same pharmaceutical composition or in different pharmaceutical compositions (at the same time or at different times). When administrated at different times, a modulator of a β2-AR regulator gene of the invention and the pharmaceutically active agent (e.g. β2-AR agonist, and/or other active agent) can be administered within 5 minutes, 10 minutes, 20 minutes, 60 minutes, 2 hours, 3 hours, 4, hours, 8 hours, 12 hours, 24 hours of administration of the other. When a modulator of a β2-AR regulator gene and the pharmaceutically active agent (e.g. β2-AR agonist, and/or other active agent) are administered in different pharmaceutical compositions, routes of administration can be different. For example, a modulator of a β2-AR regulator gene can be administered by any appropriate route known in the art including, but not limited to oral or parenteral routes, including intravenous, intramuscular, subcutaneous, transdermal, airway (aerosol), pulmonary, nasal, rectal, and topical (including buccal and sublingual) administration, and pharmaceutically active agent (e.g. β2-AR agonist, and/or other active agent) is administered by a different route, e.g. a route commonly used in the art for administration of said pharmaceutically active agent.

In some embodiments, a modulator of a β2-AR regulator gene may precede, be co-current with and/or follow the pharmaceutically active agent (e.g. β2-AR agonist, and/or other active agent) by intervals ranging from minutes to weeks. In embodiments where a modulator of a β2-AR regulator gene and pharmaceutically active agent (e.g. β2-AR agonist, and/or other active agent) are applied separately to a cell, tissue or organism, one would generally ensure that a significant period of time did not expire between the time of each delivery, such that the modulator of a β2-AR regulator gene and pharmaceutically active agent (e.g. β2-AR agonist, and/or other active agent) would still be able to exert an advantageously combined effect on the cell, tissue or organism.

In some embodiments, the invention contemplates the use of the compositions comprising a modulator of a β2-AR regulator gene alone, or in combination with a β2-AR agonist, and the practice of the methods described herein in conjunction with other therapies such as surgery, e.g., enlarging a sinus passage, remove obstructing bone or nasal polyps, mucosal stripping, removal of sinuses, bullectomy, lung volume reduction surgery, or lung transplantation.

Pharmaceutical Formulations

As described in detail below, the pharmaceutical compositions of the present invention comprising a modulator of a β2-AR regulator gene, e.g. an inhibitor of FDPS and/or ARRDC3 and/or CaMKK2 and/or AAEST or an activator of latrophilin 2 can be specially formulated for administration of the inhibitor alone, or in combination with one or more β2-AR agonists to a subject in solid, liquid or gel form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, a modulator of a β2-AR regulator gene can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquids such as suspensions (e.g., aqueous or non-aqueous liquid suspensions, oil-in-water emulsions, or water-in-oil liquid emulsions), solutions, and elixirs; and sterile solids (e.g., crystalline or amorphous solids) that can be reconstituted to provide liquid dosage forms.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alchols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of modulator of the β2-AR regulator gene, e.g. inhibits the degradation of a RNAi inhibitor present in the pharmaceutical composition.

As used herein, the term “administer” refers to the placement of a modulator of a β2-AR regulator gene, alone or combination with a β2-AR agonist into a subject by a method or route which results in at least partial localization of the inhibitor of a β2-AR regulator gene at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. The term “treatment” of a disease also includes providing relief from the symptoms or side-effects of the disease (including palliative treatment). For example, any reduction in inflammation, bronchospasm, bronchoconstriction, shortness of breath, wheezing, lower extremity edema, ascites, productive cough, hemoptysis, or cyanosis in subject suffering from a respiratory disorder, no matter how slight, would be considered an alleviated symptom. In one embodiment, the symptoms of a disease or disorder are alleviated by at least 5%, at least 10%, at least 20%, at least 30%, at least 40%, or at least 50%.

In methods of treatment described herein, the administration of a modulator of a β2-AR regulator gene, e.g. an inhibitor of FDPS or ARRDC3 or CaMKK2, or AAEST or activator of latrophilin 2, alone or combination with a β2-AR agonist can be for either “prophylactic” or “therapeutic” purpose. When provided prophylactically, a modulator of a β2-AR regulator gene, alone or combination with, a β2-AR agonist can be administered to a subject in advance of any symptom, e.g. a respiratory disorder, e.g. asthma attack. The prophylactic administration of a modulator of a β2-AR regulator gene alone or combination with a β2-AR agonist serves to prevent a respiratory disorder, as disclosed herein. When provided therapeutically, a modulator of a β2-AR regulator gene, e.g. an inhibitor of FDPS and/or ARRDC3 and/or CaMKK2 and/or AAEST, or activator of latrophilin 2, alone or combination with a β2-AR agonist is provided at (or after) the onset of a symptom or indication of respiratory disorder. Thus, a modulator of a β2-AR regulator gene, alone or combination with a β2-AR agonist can be provided prior to the onset of respiratory disorder, e.g., on set of an allergic respiratory disorder. In some embodiments, a modulator of a β2-AR regulator gene is administered to a subject prophylatically, and the β2-AR agonist is administered to a subject therapeutically at the time of onset of a symptom or indication of a respiratory disorder.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. The dosage may be determined by a physician and adjusted, as necessary, to suit observed effects of the treatment.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay, for example, using the β2-AR internalization assay as disclosed in the Examples herein.

The dosage of a modulator β2-AR regulator gene, alone or in combination with a β2-AR agonist can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

Aerosol Formulations

A modulator of a β2-AR regulator gene, e.g., FDPS, ARRDC3, CaMKK2, AAEST, latrophilin, alone or in combination with β2-AR agonists can be administered directly to the airways in the form of an aerosol or by nebulization. For use as aerosols, a modulator of a β2-AR regulator gene, alone or in combination with β2-AR agonists in solution or suspension may be packaged in a pressurized aerosol container together with suitable propellants, for example, hydrocarbon propellants like propane, butane, or isobutane with conventional adjuvants. A modulator of a β2-AR regulator gene, alone or in combination with β2-AR agonists can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefor, including by using many nebulizers known and marketed today. For example, an AEROMIST pneumatic nebulizer available from Inhalation Plastic, Inc. of Niles, Ill.

As is well known, any suitable gas can be used to apply pressure during the nebulization, with preferred gases to date being those which are chemically inert to a modulator of a β2-AR regulator gene and/or β2-AR agonists. Exemplary gases including, but are not limited to, nitrogen, argon or helium can be used to high advantage.

A modulator of a β2-AR regulator gene, alone or in combination with β2-AR agonists can also be administered directly to the airways in the form of a dry powder. For use as a dry powder, a modulator of a β2-AR regulator gene, alone or in combination with β2-AR agonists can be administered by use of an inhaler. Exemplary inhalers include metered dose inhalers and dry powdered inhalers. A metered dose inhaler or “MDI” is a pressure resistant canister or container filled with a product such as a pharmaceutical composition dissolved in a liquefied propellant or micronized particles suspended in a liquefied propellant. The correct dosage of the composition is delivered to the patient. A dry powder inhaler is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of a modulator of a β2-AR regulator gene, alone or in combination with a β2-AR agonist thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S, and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

Oral Dosage Forms

Pharmaceutical compositions comprising a modulator of β2-AR regulator gene, alone or in combination with β2-AR agonists can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington's Pharmaceutical Sciences, 18th ed., Mack Publishing, Easton, Pa. (1990).

Typical oral dosage forms of the compositions of the disclosure are prepared by combining the pharmaceutically acceptable salt of disclosed compounds in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, and disintegrating agents. Due to their ease of administration, tablets and capsules represent the most advantageous solid oral dosage unit forms, in which case solid pharmaceutical excipients are used. If desired, tablets can be coated by standard aqueous or nonaqueous techniques. These dosage forms can be prepared by any of the methods of pharmacy. In general, pharmaceutical compositions and dosage forms are prepared by uniformly and intimately admixing the active ingredient(s) with liquid carriers, finely divided solid carriers, or both, and then shaping the product into the desired presentation if necessary.

For example, a tablet can be prepared by compression or molding. Compressed tablets can be prepared by compressing in a suitable machine the active ingredient(s) in a free-flowing form, such as a powder or granules, optionally mixed with one or more excipients. Molded tablets can be made by molding in a suitable machine a mixture of the powdered compound moistened with an inert liquid diluent. Examples of excipients that can be used in oral dosage forms of the disclosure include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, and AVICEL-PH-105 (available from FMC Corporation, American Viscose Division, Avicel Sales, Marcus Hook, Pa., U.S.A.), and mixtures thereof. An exemplary suitable binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the disclosure is typically present in from about 50 to about 99 weight percent of the pharmaceutical composition or dosage form.

Disintegrants are used in the compositions of the disclosure to provide tablets that disintegrate when exposed to an aqueous environment. Tablets that contain too much disintegrant may swell, crack, or disintegrate in storage, while those that contain too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) should be used to form solid oral dosage forms of the disclosure. The amount of disintegrant used varies based upon the type of formulation and mode of administration, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used to form pharmaceutical compositions and dosage forms of the disclosure include, but are not limited to, agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, and mixtures thereof.

Lubricants that can be used to form pharmaceutical compositions and dosage forms of the disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL® 200, manufactured by W. R. Grace Co. of Baltimore, Md.), a coagulated aerosol of synthetic silica (marketed by Degussa Co. of Piano, Tex.), CAB-O-SIL® (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

This disclosure further encompasses lactose-free pharmaceutical compositions and dosage forms, wherein such compositions preferably contain little, if any, lactose or other mono- or di-saccharides. As used herein, the term “lactose-free” means that the amount of lactose present, if any, is insufficient to substantially increase the degradation rate of an active ingredient.

Lactose-free compositions of the disclosure can comprise excipients which are well known in the art and are listed in the USP(XXI)/NF (XVI), which is incorporated herein by reference. In general, lactose-free compositions comprise a pharmaceutically acceptable salt of an HIF inhibitor, a binder/filler, and a lubricant in pharmaceutically compatible and pharmaceutically acceptable amounts. Preferred lactose-free dosage forms comprise a pharmaceutically acceptable salt of the disclosed compounds, microcrystalline cellulose, pre-gelatinized starch, and magnesium stearate.

This disclosure further encompasses anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

For oral administration, the dosage should contain at least at least 0.1% of an inhibitor of β2-AR regulator genes, alone or in combination with β2-AR agonists inhibitor. The percentage of inhibitor of β2-AR regulator genes in these compositions may, of course, be varied and may conveniently be between about 2% to about 60% of the weight of the unit. The amount of an inhibitor of β2-AR regulator genes, alone or in combination with β2-AR agonists in such therapeutically useful compositions is such that a suitable dosage will be obtained.

Controlled and Delayed Release Dosage Forms

In some embodiments, a modulator a β2-AR regulator gene, alone or in combination with a β2-AR agonist, can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Chemg-ju, Controlled Release Dosage Form Design, 2 (Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

One embodiment of the disclosure encompasses a unit dosage form that includes a pharmaceutically acceptable salt of the disclosed compounds (e.g., a sodium, potassium, or lithium salt), or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the disclosure. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the disclosure include, but are not limited to, the OROS® Push-Pull™, Delayed Push-Pull™, Multi-Layer Push-Pull™ and Push-Stick™ Systems, all of which are well known. See, e.g. worldwide website alza.com. Additional OROS® systems that can be used for the controlled oral delivery of compounds and compositions of the disclosure include OROS®-CT and L-OROS®; see, Delivery Times, vol. II, issue II (Alza Corporation).

Conventional OROS® oral dosage forms are made by compressing a drug powder (e.g., an inhibitor of a β2-AR regulator gene which is a salt) into a hard tablet, coating the tablet with cellulose derivatives to form a semi-permeable membrane, and then drilling an orifice in the coating (e.g., with a laser). Kim, Chemg-ju, Controlled Release Dosage Form Design, 231-238 (Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively delivery drugs with low water solubility.

A specific dosage form of a modulator of a β2-AR regulator gene, alone or in combination with a β2-AR agonist, compositions of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent the exit orifice and in direct or indirect contacting relationship with the expandable layer; and a flow-promoting layer interposed between the inner surface of the wall and at least the external surface of the drug layer located within the cavity, wherein the drug layer includes a modulator of a β2-AR regulator gene, alone or in combination with a β2-AR agonist, or a polymorph, solvate, hydrate, dehydrate, co-crystal, anhydrous, or amorphous form thereof. See U.S. Pat. No. 6,368,626, the entirety of which is incorporated herein by reference.

Another specific dosage form of the disclosure includes: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a modulator of a β2-AR regulator gene, or a polymorph, solvate, hydrate, dehydrate, co-crystal, anhydrous, or amorphous form thereof. See U.S. Pat. No. 6,342,249, the entirety of which is incorporated herein by reference.

In some embodiments, a modulator of a β2-AR regulator gene, alone or in combination with a β2-AR agonist is administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administration are particularly preferred when the respiratory disorder occurs continuously in the subject, for example where the subject has continuous symptoms of a respiratory disorder. Each pulse dose can be reduced and the total amount of drug administered over the course of treatment to the patient is minimized.

Individual pulses can be delivered to the patient continuously over a period of several hours, such as about 2, 4, 6, 8, 10, 12, 14 or 16 hours, or several days, such as 2, 3, 4, 5, 6, or 7 days, preferably from about 1 hour to about 24 hours and more preferably from about 3 hours to about 9 hours.

The interval between pulses or the interval of no delivery is greater than 24 hours and preferably greater than 48 hours, and can be for even longer such as for 3, 4, 5, 6, 7, 8, 9 or 10 days, two, three or four weeks or even longer. As the results achieved may be surprising, the interval between pulses, when necessary, can be determined by one of ordinary skill in the art. Often, the interval between pulses can be calculated by administering another dose of the composition when the composition or the active component of the composition is no longer detectable in the patient prior to delivery of the next pulse. Intervals can also be calculated from the in vivo half-life of the composition. Intervals may be calculated as greater than the in vivo half-life, or 2, 3, 4, 5 and even 10 times greater the composition half-life.

The number of pulses in a single therapeutic regimen may be as little as two, but is typically from about 5 to 10, 10 to 20, 15 to 30 or more. In fact, patients can receive drugs for life according to the methods of this invention without the problems and inconveniences associated with current therapies. Compositions can be administered by most any means, but are preferable delivered to the patient as an injection (e.g. intravenous, subcutaneous, intraarterial), infusion or instillation. Various methods and apparatus for pulsing compositions by infusion or other forms of delivery to the patient are disclosed in U.S. Pat. Nos. 4,747,825; 4,723,958; 4,948,592; 4,965,251 and 5,403,590. Sustained release can also be accomplished by means of an osmotic pump. In some embodiments, an inhibitor of β2-AR regulator gene is administered over a period of several days, such as 2, 3, 4, 5, 6 or 7 days.

Parenteral Dosage Forms

Parenteral dosage forms of a modulator of a β2-AR regulator gene, alone or in combination with a β2-AR agonist can also be administered to a subject with a respiratory disorder by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Compounds that alter or modify the solubility of a pharmaceutically acceptable salt of a modulator of a β2-AR regulator gene, alone or in combination with a β2-AR agonist as disclosed herein can also be incorporated into the parenteral dosage forms of the disclosure, including conventional and controlled-release parenteral dosage forms.

Topical, Transdermal and Mucosal Dosage Forms

Topical dosage forms of the disclosure include, but are not limited to, creams, lotions, ointments, gels, shampoos, sprays, aerosols, solutions, emulsions, and other forms known to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well known in the art. See, e.g., Remington's Pharmaceutical Sciences, 18.sup.th Ed., Mack Publishing, Easton, Pa. (1990).

Transdermal and mucosal dosage forms of the compositions comprising a modulator of a β2-AR regulator gene, alone or in combination with a β2-AR agonist as disclosed herein include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the active ingredient(s) of the disclosure include, but are not limited to, those disclosed in U.S. Pat. Nos. 4,624,665; 4,655,767; 4,687,481; 4,797,284; 4,810,499; 4,834,978; 4,877,618; 4,880,633; 4,917,895; 4,927,687; 4,956,171; 5,035,894; 5,091,186; 5,163,899; 5,232,702; 5,234,690; 5,273,755; 5,273,756; 5,308,625; 5,356,632; 5,358,715; 5,372,579; 5,421,816; 5,466,465; 5,494,680; 5,505,958; 5,554,381; 5,560,922; 5,585,111; 5,656,285; 5,667,798; 5,698,217; 5,741,511; 5,747,783; 5,770,219; 5,814,599; 5,817,332; 5,833,647; 5,879,322; and 5,906,830, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with a modulator of a β2-AR regulator gene. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of a modulator of β2-AR regulator gene can be used to further adjust the properties of the resulting composition.

### In Vitro Functional Assay for Determining β2-Adrenergic Receptor Function

The β2-adrenergic receptor functional activity of β2-AR agonists in an admixture also comprising a modulator of a β2-AR regulator gene of the invention can be tested using the in vitro assay as disclosed in the Examples, or alternative assays as follows.

Cell Seeding and Growth: Primary bronchial smooth muscle cells from a 21 yr. old male (Clonetics, San Diego Calif.) were seeded at 50,000 cells/well in 24-well tissue culture plates. The media used was Clonetic's SmBM-2 supplemented with hEGF, Insulin. hFGF, and Fetal Bovine Serum. Cells were grown two days at 37° C., 5% CO, until confluent monolayers were seen.

Agonist Stimulation of Cells

The media was aspirated from each well and replaced with 250 ml fresh media containing 1 mM IBMX, a phosphodiesterase inhibitor (Sigma, St Louis, Mo.). Cells were incubated for 15 minutes at 37° C., and then 250 ml of agonist at appropriate concentration was added. Cells were then incubated for an additional 10 minutes. Media was aspirated and 500 ml cold 70% EtOH was added to cells, and then removed to an empty 96-well deep-well plate after about 5 minutes. This step was then repeated. The deep-well plate was then spun in a speed-vac until all EtOH dried off, leaving dry pellets. cAMP (pmol/well) was quantitated using a cAMP ELISA kit from Stratagene (La Jolla, Calif.). EC 50 curves were generated using the 4-parameter fit equation: y=(a−d)/(1+(x/c)b)+d, where, y=cpm a=total binding c=IC 50; x=[compound] d=NS binding b=slope. Fix NS binding and allow all other parameters to float.

β2-Adrenergic Receptor In Vitro Radioligand Binding Assay. The β1/2-adrenergic receptor binding activity of compounds of the invention can be tested follows. Incubating SF9 cell membranes containing either β1 or β2-adrenergic receptor (NEN, Boston, Mass.) with 0.07 nM 121 I-iodocyanopindolol (NEN, Boston, Mass.) in binding buffer containing 75 mM Tris-HCl (pH 7.4), 12.5 mM MgCl, and 2 mM EDTA and varying concentrations of test compounds or buffer only (control) in 96-well plates. Incubating the plates at room temperature with shaking for 1 hour. The receptor bound radioligand can be harvested by filtration over 96-well GF/B filter plates (Packard, Meriden, Conn.) pre-blocked with 0.3% polyethylenimine and washed twice with 200 μl PBS using cell harvester. The filters were washed three times with 200 μl PBS using cell harvester and then resuspended in 40 μl scintillation cocktail. The filter-bound radioactivity was measured with a scintillation counter and IC 50 curves are generated using the standard 4-parameter fit equation described above.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

### EXAMPLES

The examples presented herein relate to admixture compositions comprising at least one β2-AR agonist and at least one agent which inhibits a β2-AR regulator gene, e.g. an agent which inhibits at least one of FDPS (farnesyl diphosphate synthase), and/or ARRDC3 (arrestin domain containing 3) and/or CaMKK2, and/or AAEST and/or latrophilin 2, and uses thereof, for example for the treatment of respiratory disorders in a subject. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Materials and Methods

Enzymatic Conversion of cDNA into shRNA Expressing Library. For shRNA library targeting specific genes, the cDNA fragments of coding regions were amplified by PCR and purified from agarose gel. For EST library-derived shRNA expressing library, the EST fragments were prepared as described previously (Lu, 2004 #48). The inventors then converted the cDNA or EST fragments into shRNA expressing libraries according to the following five-step procedure. DNA restriction enzymes, polymerases, ligases and other reagents were from New England Biolab (NEB) otherwise was mentioned separately.

Step 1. cDNA fragmentation. 2.5 μg of cDNA fragments were partially digested with 0.2 U DNase I at 25° C. for 10-15 min. The optimal digestion time was empirically determined so that the average size of final digests was about 100-300 nt. The ends of the digests were repaired with 5 U T4 DNA polymerase and 200 μM dNTPs. The addition of an adenine to 3′ ends (3′-A overhang) was done by using 1 U Taq polymerase and 200 μM dATP and incubating at 60° C. for 3 h.

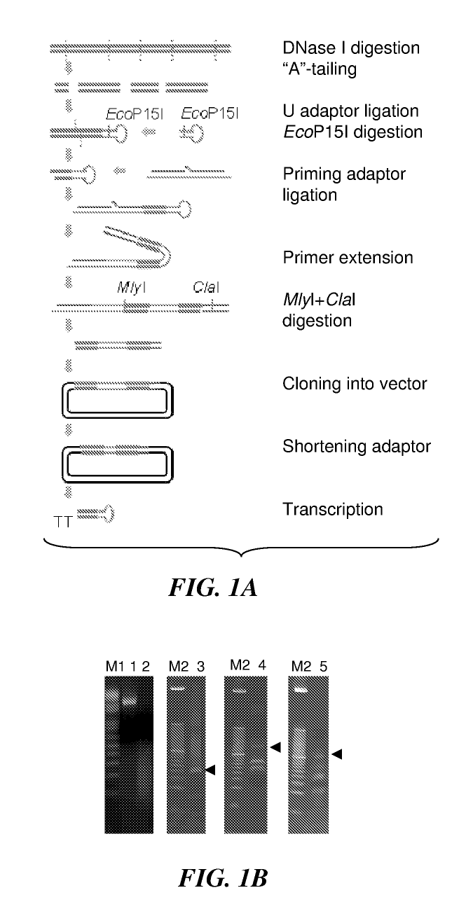

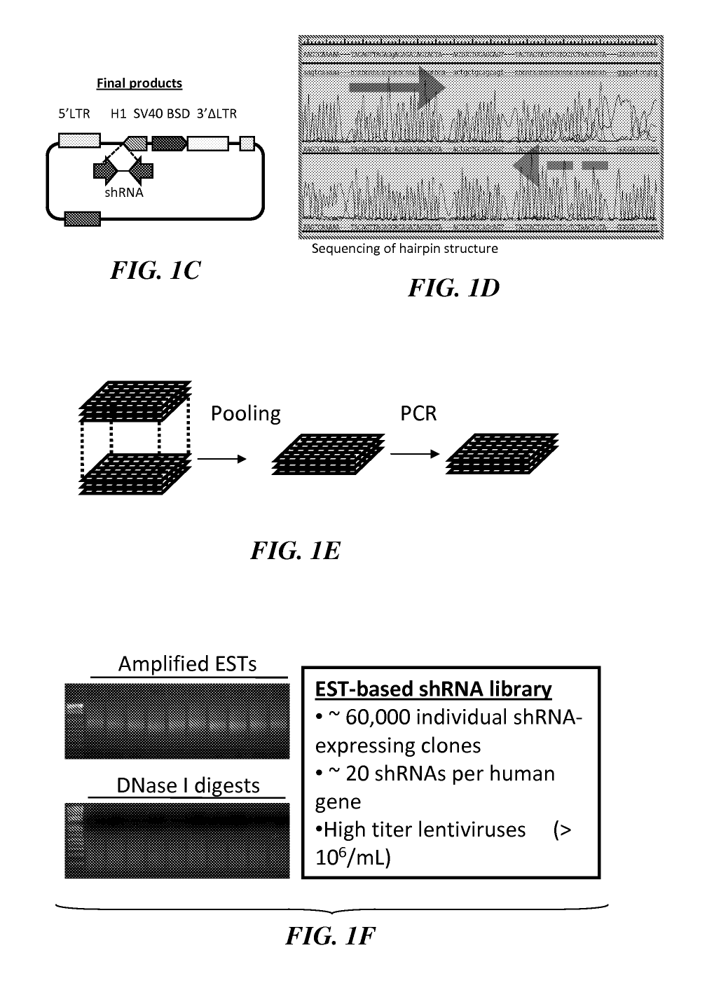

Step 2. U adaptor ligation and EcoP15I cleavage. The digests with 3′-A overhang were ligated to 50 pmol of a hairpin-shaped oligonucleotide, U adaptor (5′-pCTG CTG CAG GAT CCA AAT CCT GCA GCA GT-3′ (SEQ ID NO: 6)) which was PAGE-purified, 5′-phosphorylated and self-annealed before use. The adaptor has overlapping recognition sequences for PstI (CTGCAG) (SEQ ID NO: 7) and EcoP15I (CAGCAG) (SEQ ID NO: 8). The ligation product was digested with EcoP151 and the resulting U adaptor-linked short DNA fragments were purified as ˜40 bp species by PAGE (FIG. 1B lane 3).

Step 3. Priming adaptor ligation and primer extention. The U adaptor-linked short DNA fragments were ligated with a priming adaptor comprised of three oligonucleotides (10 pmol of each): a priming oligo (in FIG. 1A, 5′-ACA TTT TGC TGC CGG TC-3′) (SEQ ID NO: 8); a ligation sense oligo (purple line, 5′-GGA TCG ATA AGT CAA AAA-3′ (SEQ ID NO: 9)); and a ligation antisense oligo (green line, 5′-pNNT TTT TGA CTC ATC GAT GGG ACC GGC AGC AAA ATG TTC G-3′ (SEQ ID NO: 10), 5′-phosphorylated PAGE-purified). Both the sense and antisense ligation oligos contain the recognition site for ClaI (ATCGAT) (SEQ ID NO: 11) while only the antisense ligation oligo has the recognition site for MlyI (GAGTC, antisense mutated to: GACTC) (SEQ ID NO: 12). The ligation products were then converted into palindromic dsDNAs by primer extension and stand displacement with 1 U Bst DNA polymerase large fragment and 200 ng single-stranded DNA binding Protein. The extension products were identified as ˜140 bp species by PAGE (FIG. 1B lane 4).

Step 4. Cloning into the lentiviral vector. The extension products were digested with Mlyl which moved all linker sequence and leaved a blunt end at one side, and ClaI which leaved with a short linker sequence of RNA Polymerase III stop signal TTTTT (SEQ ID NO: 13) and ClaI site overhang at the other side. The digested products were purified as ˜100 bp species by PAGE (FIG. 1B lane 5) and ligated with SmaI and ClaI digested vector pLentisuper2. The cloning site SmaI were placed just downstream from the H1 promoter to allow transcription starting right from 5′-end of the 26-bp palindrome (Brummelkamp, 2002 #55). The ligation products were transformed into the ultra-competent bacteria XL-10 Gold (Stratagene, La Jolla, Calif., USA) and plasmid DNA was purified.

Step 5. Truncation of excessive hairpin linker. Plasmid DNA was digested with PstI to remove most of the U adaptor sequence. The digested plasmid DNA was purified by agarose gel and recircularized by self-ligation. Ligation products were transformed into ultra-competent bacteria XL-10 Gold and plasmid DNA was purified as library stocks.

DNA Vectors. The backbone vector of our lentiviral shRNA expressing library is pLentiSuper (Invitrogen). It expresses Blasticidin resistant gene from the SV40 promoter and shRNA form the RNA Polymerase III H1 promoter, The H1 promoter is in the opposite orientation to the 5′-LTR promoter with downstream cloning sites BamHI and ClaI. To facilitate cloning of blunt end inserts, a SmaI site was introduced by insertion of a linker between BamHI and ClaI (sense 5′-GAT CCC CGG GCA CAC AAT-3′ (SEQ ID NO: 14), antisense 5′-CGA TTG TGT GCC CGG G-3′ (SEQ ID NO: 15)). The lentiviral vector was called pLentiSuper2. Other DNA vectors include pEGFP-C1 expressing EGFP gene (Clontech); pEGFP-N1-ARRDC1 expressing the human ARRDC1 with C-terminal fusion of EGFP; pcDNA3-FLAG-SMURF2 (provided by Dr. Hong. Zhang, University of Massachusetts Medical School, Worcester, Mass., USA) and pcDNA3-FLAG-UEV3 expressing FLAG-tagged human SMURF2 and FLAG-tagged human UEV3 respectively.

Cell Culture, Transfection and Drug Treatments. Human renal epithelial cell line 293T, 293.β2AR* and 293.β2AR^{WT} were cultured in DMEM (Invitrogen) containing 10% fetal bovine serum (FBS, Invitrogen), 4 mM Glutamine, 100 Units/mL penicillin and 100 μg/mL streptomycin. 293. β2AR* and 293.β2AR^{WT} are sub-lines of HEK293 expressing N-terminal FLAG-tagged, recycle-deficient β2AR and N-terminal FLAG-tagged, wild type β2AR respectively. Transfection of DNA into 293T cells was using FuGene 6 reagent (Roche) according to manufacturer's instruction. Transfection of siRNA into 293.FLAG-β2AR* cells was using DharmaFECT 1 reagent (Thermo Scientific). FDPS-specific siRNA (sense 5′-cca ugu aca ugg cag gaa u(dT)(dT)-3′, antisense 5′-auu ccu gcc aug uac aug g(dT)(dT)-3′) was from Sigma-Aldrich and the non-targeting Scrambled siRNA was from Thermo Scientific. Isoproterenol and alendronate were from Sigma-Aldrich and dissolved in water as stocks, which were then diluted in 5% volume of culture medium and added dropwise to cell cultures.

Lentivirus production and transduction. Recombinant lentiviruses were produced by a 3-plasmid transfection procedure. Briefly, 293T cells were co-transfected using FuGene 6 reagent and the pLentiSuper2 backbone lentiviral vector together with the vector encoding packaging proteins Gag-Pol and Rev and the vector encoding G-protein of vesicular stomatitis virus (VSVG). The virus supernatants were collected 24, 36, 48 and 60 hours after transfection, pooled and filtered through 0.45-μm filters. Viral titers were determined by the number of Blasticidin-resistant cell colonies transduced with serial dilutions of lentivirus supernatants. For transduction of the shRNA library, 1×10⁷ 293.FLAG-β2AR* cells were seeded in 10-cm tissue culture dishes (2.5×10⁶ per dish). 24 h later, each dish of cell were infected with 1×10⁷ pfu lentivirus of the shRNA library in medium containing 5 μg/mL polybrene (Sigma-Aldrich). 2 d post transduction, cells were selected in medium containing 5 μg/mL blasticidin for 8 d.

FACS-based shRNA Screen and Flowcytometry. 5×10⁶ shRNA library transduced 293.FLAG-β2AR* cells were culture in regular medium for 1d then in Opti-MEM (Invitrogen) containing 1% FBS and 10 μM isoproterenol for 16 h to induced the internalization of β2AR. Cells were washed with PBS and incubated with diluted TrypLE Express Enzyme (Invitrogen) (1:10 dilutio in PBS) for exactly 1 min at room temperature followed by adding regular medium immediately. Cells were washed once with PBS containing 1% FBS to remove any trace of TrypLE. 1×10⁷ cells were suspended in 1 mL PBS containing 1% FBS and stained with 10 μg/mL FITC-conjugated anti-FLAG M2 antibody (Sigma-Aldrich) on a rotary mixer for 30 min. Then cells were washed again and passed through cell strainers to remove cell clumps before being sorted on a FACS Aria multicolor high speed sorter (Becton Dickinson). During FACS analysis, the population of single cell was gated to measured FITC (green) signal. Background of auto-fluorescence was identified by plotting FITC with PE (red) or SSC (side scatter) signal. FITC-positive cells were collected in calcium and magnesium-free PBS with 20% FBS. Collected cells were cultured for repeating FACS sorting. About 7.8×10⁴, 1.5×10⁵, 2.8×10⁵, and 1.4×10⁶ FITC-positive cells were collected in 4 rounds of sequential sorting, which corresponded to about 1%, 3%, 10% and 29% of the of single cell population. Flowcytometric analysis used similar procedures as FACS except that only 1×10⁶ cells were used for each sample and counterstained with 2 μg/mL propidium iodide (PI) before being analyzed on a FACSCalibur analyzer system (Becton Dickinson). The population of PI-negative single cell was gated to measured FITC signal.

Subcloning and Sequencing of shRNA Expressing Cassettes. Genomic DNA was isolated by using Gentra Puregene Cell Kit (Qiagen). PCR was performed using 200 ng genomic DNA, 0.5 μM of primers H1-2F (5′-CAG GAA GAT GGC TGTGAG GGA C-3′ (SEQ ID NO: 16)) and H1-4R (5′-CGG ATC TCG ACG GTA TCG GT-3′) (SEQ ID NO: 17), 5 U Taq polymerase (NEB) and 0.25 U Pfu polymerase (Stratagene), 200 μM of each dNTP and 1×Pfu buffer in total of 50 μL volume. The 83-bp shRNA expressing sequences were released from the 362-bp PCR products by digestion with BamHI and ClaI, gel purified and inserted into the BamHI and ClaI sites on pLentiSuper2. BDX Chemistry (Sequetech) was used in DNA sequencing with primers H1-1F (5′-CTT TGG ATT TGG GAA TCT TA-3′) (SEQ ID NO: 18) and/or H1-1R (5′-GCA ACA GAC ATA CAA ACT AAA G-3′) (SEQ ID NO: 19).

Real-Time RT-PCR. Total RNA was isolated by using TRIzol (Invitrogen) and further cleaned up by using RNeasy Mini kit (Qiagen) with DNase I digestion. cDNA was synthesized by using oligo-dT primers and SuperScript III (Invitrogen). Real-time PCR was performed on ABI Prism 7300 with SYBR Green (Qiagene). The FDPS was amplified with primers FDPS-1F (5′-TGA CCG GCA AAA TTG GCA CTG ACA T-3′) (SEQ ID NO: 20) and FDPS-1R (5′-CCT TCA CCC GGG CCA CTT TCT CA-3′) (SEQ ID NO: 21). The β-Actin control was amplified with appropriate primers. The β-Actin control was amplified with primers (Forward, 5′-AAG GCC AAC CGC GAG AAG-3′ (SEQ ID NO: 28) and Reverse, 5′-GGC GTA CAG GGA TAG CAC-3′ (SEQ ID NO: 29)).

Immunoblot analysis. 48 h post DNA transfection, cells were wash with PBS and lysed with NP-40 lysing buffer (50 mM Tris-HCl pH7.5, 150 mM NaCl, 0.5% Nonidet P-40) containing protease inhibitor cocktail (Roche). Protein samples were separated on NuPAGE 10% Bis-Tris gels (Invitrogen) and transferred to nitrocellulose membranes (Bio-Rad). Immunoblot signals were developed using SuperSignal West Pico Chemiluminescent Substrate (Pierce, Rockford, Ill., USA) and quantified by a Luminescence Imager or exposed to X-ray films. Horseradish peoxidase (HRP)-conjugated mouse anti-FLAG M2 antibody was from Sigma-Aldrich, rabbit antibody for GFP was from Clontech, and HRP-conjugated goat anti-rabbit IgG antibody was from Zymed Laboratories.

β2AR ubiquitination and degradation assays. Cells expressing PAR or β2ARt were subjected to 10 μM ISO (Sigma) stimulation, lysed in 0.5% NP-40 lysis buffer and immunoblotted with anti-FLAG-HRP (Sigma). For β2AR ubiquitination, β2ARt or wt-β2AR expressing cells were first incubated with 10 μM alprenolol antagonist (Sigma) for 15 min (to reduce constitutive background ubiquitination), stimulated with 10 μM ISO and lysed in buffer supplemented with 10 mM N-ethylmaleimide (NEM; Sigma) to inhibit deubiquitinase activity. Anti-FLAG EZview beads (Sigma) used for the IP were subjected to three 10 min washes with lysis buffer and resuspended in 50 μl 2βLDS sample buffer (Invitrogen) containing 10% β-mercaptoethanol (Sigma) and analyzed by immunoblotting.

Co-immunoprecipitations (co-IP). 293T cells were transfected using Fugene 6 (Roche) or Turbofect (Fermentas). 48 h later, cells were lysed, pre-cleared with protein A agarose, and HA- or FLAG-tagged proteins were IP-ed using anti-HA EZview or anti-FLAG EZview beads. Washed beads were resuspended in sample buffer and analyzed by immunoblotting. Where noted, cross-linking was achieved by incubation for 40 min with 2 mM crosslinker dithio-bismaleimidoethane (DTME; Pierce) in PBS containing 10 mM Hepes (pH 7.5), as previously described (Shenoy et al, 2008) with minor modifications. IP complexes were washed three times consecutively in lysis, high salt, and low salt buffers then resuspended in sample buffer before immunoblotting.

Immunofluorescence assays and confocal microscopy. β2AR-expressing cells seeded on glass cover slips (VWR) were transfected then incubated with 10 μg/ml anti-FLAG M2 polyclonal or monoclonal antibodies (Sigma) before stimulation with ISO. Cells were washed, fixed with 3.8% paraformaldehyde and permeabilized with 0.1% TritonX-100 buffer in TBS supplemented with 3% bovine serum albumin (BSA). Primary and secondary antibody incubations were done as indicated before visualization. Visualization and image acquisition were carried out using a Leica TCS-NT laser scanning confocal microscope (Leica) fitted with air-cooled argon and krypton lasers.

Cellular cholesterol measurement. 1×10⁶ cells were cultured in each well of 6-well plates in DMEM containing 10% FBS for 3 d until fully confluent. Then drug treatments were performed in either DMEM containing 10% FBS or Opti-MEM without serum in duplicates. Cells were transferred to microcentrifuge tubes, washed twice with ice-cold PBS. Total lipids were extracted by resuspending and lysing cells in 300 μL of Chloroform: Isopropanol: Triton X-100 (7:11:0.1). The organic phase was collected after centrifugation at 12,000 rpm for 10 min and then evaporated at 50° C. for 3-4 h. Lipids were resuspend in 200 μL of Cholesterol Reaction buffer included in a commercial cholesterol measurement kit (Invitrogen) by votexing. Cholesterol was measured by the cholesterol oxidase method according to the manufacturer's directions. 2 or 4 measurements were taken for each replicate sample.

Immunofluorescence staining and confocal imaging. Visualization of endogenous β2AR was carried out using indirect fluorescence microscopy in FDPS-knockdown and control HASM cells. Cells were cultured on glass cover slips and growth medium were removed by washing cells twice with PBS. Fixation and permeabilization of cells was done simultaneously by incubation with freshly dissolved 4% paraformaldehyde and 0.1% Triton X-100 in PBS for 30 min, followed by incubation in fresh 50 mM NH₄Cl in PBS for 15 min to quench the remaining paraformaldehyde and washes with PBS. Nonspecific bindings were pre-blocked with 5% goat serum in washing buffer (PBS plus 1% bovine serum albumin and 0.1% Triton X-100) for 15 min. Cells were then incubated with 4 μg/mL of rabbit anti-β2AR antibody (Santa Cruz) in washing buffer for 1 h and washed with washing buffer three times. Then cells were incubated with 10 μg/mL of goat anti-rabbit IgG Alexa Fluor 647 conjugated antibody (Invitrogen) in washing buffer for 45 min and washed with washing buffer three times. After cover slips were mounted on glass slides, immuofluoresence was examined using a Leica TCSNT laser confocal microscope in Harvard NIEHS Center for Environmental Health Biomedical Imaging Facility.

Mechanical Properties. Mechanical properties of the living cells were assessed using Optical Magnetic Twisting Cytometry (OMTC), a technique described in detail elsewhere (Fabry, 2003 #401), where a living cell is sheared between the plate upon which it is adhering and a ferrimagnetic microbead. Living cells were serum-deprived and incubated with insulin and transferrin for 36 hrs prior each measurement. The cells were plated overnight at confluence in collagen I-coated plastic wells (96-well Removawells, Immunlon, IL). Before each measurement, microbeads were added to the well and incubated for 20 min. The microbeads, typically 4.5 μm in diameter, were coated with a peptide containing the sequence RGD, which binds specifically to integrin proteins at the apical surface of the cell and, therefore, the actin cytoskeleton (Fabry, 2003 #401). Using an external twisting field, a magnetic microbead applies a torque on the cell, causing the microbead to move back and forth, and deform the cell. The deformation of the cell is measured as the displacement of the microbead with an inverted microscope; the stress was inferred from the applied magnetic torque. The elastic modulus is defined as the ratio, in Fourier space, of the applied torque per microbead volume and the microbead displacement. The elastic modulus is expressed as a stress per units of displacement (Pascal per nanometer); the higher the elastic modulus, the stiffer the cell. For each well, the displacement of nearly one hundred microbeads could be simultaneously recorded.

### Example 1

### Development of a Novel shRNA Library Derived from ESTs

Until recently, characterization of gene functions required laborious, individually focused investigation. Current developments in genome-wide RNAi screens allow discovery of candidate genes of essential functions in both physiological and pathological functions. While widely used, current RNAi methods have their own drawbacks. One type of most common RNAi libraries use chemically synthesized DNA oligonucleotides on expression vectors to express short hairpin RNAs (shRNAs). Construction of such libraries is laborious, costly, and error-prone. Moreover, the resulting libraries often contain a limited number of targeting shRNAs (usually 3-5) for each single gene. The targeting shRNA sequences are usually chosen based on computer algorithms that predict the knockdown efficiency of shRNAs. These algorithms vary in sequence prediction and are far from perfect. Thus, many of the genes in those libraries may not have effective targeting shRNAs. One way to address these issues and increase the number of targeting shRNAs per gene is to enzymatically generate shRNA expressing sequences from cDNAs. Previously, several laboratories have reported enzyme-mediated methods for generating numerous functional shRNA expressing constructs from DNAs that code for genes of interest or a pool of genes (Shirane, 2004 #2; Kittler, 2007 #107; Du, 2006 #8; Buchholz, 2006 #1).

Disclosed herein, inventors have discovered a method for enzymatic preparation to produce a novel EST-derived shRNA library, and its application in the first genome-wide screen for genes regulating agonist-induced internalization of β2AR. The inventors discovered farnesyl diphosphate synthase (FDPS), a key enzyme in the mevalonate pathway, and the pharmaceutical target for diseases of bone resorption, as a novel regulator in agonist-induced β2AR internalization.

Accordingly, the inventors have established and demonstrated a much improved, efficient procedure (FIG. 1A) that converts cDNAs, in an unbiased fashion, into structures expressing 26-nucleotides (nt) long shRNAs. Such longer shRNAs exhibit more potent knockdown activity than regular 19-21 nt long shRNAs (Siolas, 2005 #216), (Kim, 2005 #314). This approach takes advantage of a type IIS restriction enzyme EcoP15I that cuts about 24 and 26-nt away from its recognition site (Moncke-Buchner, 2004 #316). Hairpin-shaped adaptors were designed with an imbedded EcoP15I recognition site and ligated it to randomly fragmented cDNA sequences. Cutting with EcoP15I generated hairpin-shaped DNA fragments of 26 base pairs (bp) cDNA sequence linked to the adaptor. The inventors used primer extension and stand displacement to convert the hairpin constructs into palindromic double-stranded DNAs (dsDNAs). Cloning into lentiviral vector and subsequent expression of palindromic dsDNAs from the polymerase III promoter (H1) leads to production of target cDNA specific 26-nt long shRNAs (FIGS. 1A and B)

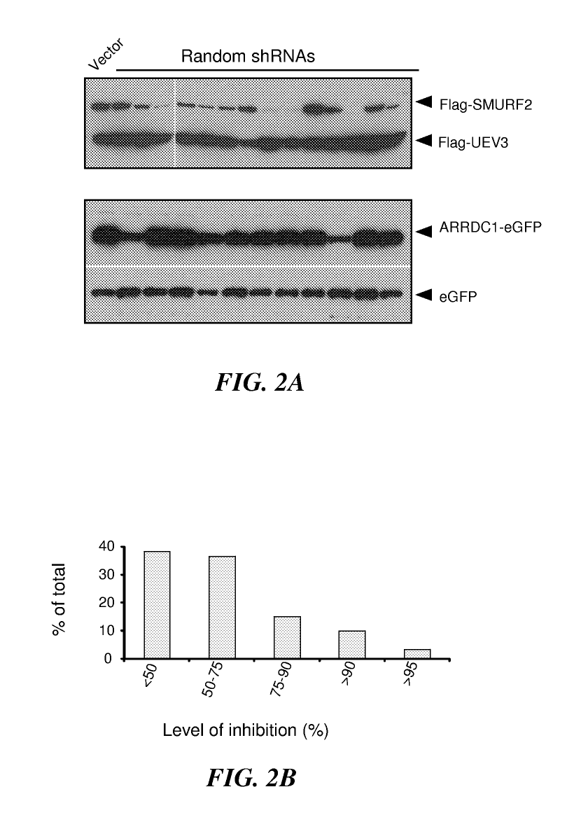

To validate the method for producing functional shRNAs, the inventors constructed single gene shRNA libraries for human SMURF2 and ARRDC1. The inventors then randomly picked shRNA plasmid clones from the libraries and tested their knockdown efficiency by immunoblot analysis. The inventors co-transfected each shRNA expressing clone or the empty vector into 293T cells together with a target gene and a non-target control. Several randomly selected shRNA clones down regulated the target protein efficiently while they had no or little effect on non-target protein (FIG. 2A). The inventors compared total of 60 individual shRNA expressing clones for ARRDC1 to the control empty vector by densitometric measurement on immunoblots. The inventor demonstrated that over 20% of shRNAs reduced the expression of target protein by at least 75%, and about 10% of shRNAs had more than 90% inhibition on the expression of target protein (FIG. 2B).

Thus, using the enzymatic conversion procedure, the inventors have demonstrated and constructed a comprehensive, genome-wide shRNA library for a large collection of over 40,000 mostly unique EST (expressed sequence tag) sequences. These ESTs correspond to about 28,000 human genes (Lu, 2004 #48). The library contains an estimated 600,000 individual 26-bp long shRNA expressing constructs. This translates into over 30 different shRNAs for an average gene in the library. Based on the data obtained for single gene shRNA libraries, we estimate that there are about 6 efficient shRNA constructs (with >75% target inhibition) for each gene in the library. After converting these ESTs into shRNA expressing structures and cloning them into vector pLentiSuper2, the inventors obtained about 600,000 bacterial colonies (individual shRNA expressing clones). This translates into over 20 different shRNAs for an average gene in the original EST collection. Based on the data obtained for the single gene shRNA libraries, we estimate that more than 20% of the randomly generated shRNAs can produce efficient knockdown (>75% inhibition). Therefore, there are at least 4 very efficient shRNA constructs for each of the genes in the library. Therefore, we have established a unique genome-wide gene inactivation library that contains diverse, highly potent 26-nt shRNAs.

### Example 2

### Genome-Wide Screen for Genes Regulating Agonist Induced Internalization of β2AR

Herein, the inventors demonstrate the establishment of a robust assay system to identify target genes which serve as β2-AR regulator genes, and where modulation (e.g., inhibition or activation) of such β2-AR regulator genes serves to prevent β2-AR degredation.

Most in vitro cell lines expressing β2AR exhibit slow receptor degradation. For example, in most cell culture models, including cells expressing exogenous β2AR, β-agonist stimulation leads to a very modest reduction in the receptor protein level (Liang, 2003; Moore, 1999). The slow degradation is attributed to quick recycling of the receptor back to the cell surface after internalization (Cao, 1999; Gage, 2001; Hanyaloglu, 2007; Xiang, 2003). Von Zastrow and colleagues found that modifications of β2AR at the C-terminus interfere with the recycling process and result in much faster receptor degradation upon agonist stimulation (Gage, 2001; Cao, 1999). The inventors have obtained an HEK293 cell line (designated as 293β2AR*) that expresses such a 2AR construct. The modified β2AR is N-terminally Flag-tagged and thus can be easily detected using anti-FLAG antibodies (von Zastrow, 1992).

The β2AR undergo internalization upon binding of its agonists, a process known as receptor desensitization (Hanyaloglu, 2008; Moore, 2007). The internalized β2ARs first localize in early endosomes, most of which recycle back to the cell surface (desensitization), while very few fuse with lysosomes for degradation. These receptor desensitization and resensitization processes are of very importance in maintenance of tissue homeostasis. However, prolong agonist exposure results in redirecting the internalized β2ARs from recycling to degradation, which may limit the efficacy of long term β2-agonist treatment (Tsao, 2000; Tsao, 2001).

In most cell culture models, including cells expressing exogenous β2AR, agonist stimulation leads to a very modest reduction in the receptor protein level (Liang, 2003; Moore, 1999). For example, overnight induction with β2AR-agonist isoproterenol at a relatively high concentration results in less than 50% degradation of the receptor (Moore, 1999). Cells exhibiting such slow and inefficient degradation are unsuitable for high-throughput genetic screens. To circumvent this problem, the inventors have characterized a cell line (293β2AR*) that expresses a modified version of β2AR. The inventors have clearly demonstrated that upon agonist stimulation the modified β2AR degrades much faster than unmodified β2AR. The inventors also demonstrated that such cells were suitable for FACS-based assay, and can be used for fluorescent labeling to quantify the cell surface β2AR. With such high-throughput assay, the inventors are able to carry out genome-wide shRNA screen to identify human genes involved in β2AR regulation.

The inventors have also used a genome-wide shRNA library which has several key advantages over existing RNAi libraries. Firstly, it uses lentiviral vector that can facilitate the transduction of shRNA library into different cell types (Brummelkamp, 2002). Secondly, the shRNA sequences were derived from an EST collection in which the abundance of every sequence was normalized equally (Lu, 2004). Thirdly, the shRNA expressing sequences contained long (26-bp) target gene sequences, which was known much more efficient in gene silencing as compared to the widely used 19 to 21-bp shRNAs (Siolas, 2005; Kim, 2005). Lastly, the library has about 20 shRNA sequences for each average human gene, which is much higher than 3-5 per gene in other similar libraries.

Using our novel EST-derived shRNA expressing library, the inventors have identified several potential regulators for β2AR internalization and degradation. Some of them have predicted function in vesicle trafficking, while some have known functions in lipid biosynthesis. A higher coverage of the genome could be achieved by increasing the size of the shRNA expressing library and/or isolating more shRNA clones after a genetic screen. Interestingly, one potential regulator, ARRDC3 showed very high structure similarity to a family of proteins, Arrestins that regulate GPCR internalization (Marchese, 2008 #312; Moore, 2007 #313). As demonstrated in Example 4, the inventors have confirmed ARRDC3 promotes β2AR degradation by regulating its ubiquitination (to be published separately), and that inhibition of ARRDC3 increases β2-AR on the cell surface and prevents its internalization and subsequent degradation. Therefore, the inventors have demonstrated herein the use of the genome-wide shRNA library for use in a screen to identify valid regulators for β2AR function.

The inventors next transduced 293β2AR* cells with the EST-derived shRNA lentiviral library to establish a cellular shRNA library. The inventors then seeded total 10 million cells, cultured them for one day and then transduced them with 40 million infectious titers of shRNA library viruses. Therefore, the size of cellular library, which was determined by the number of viral infection events, could be more than 10 million.

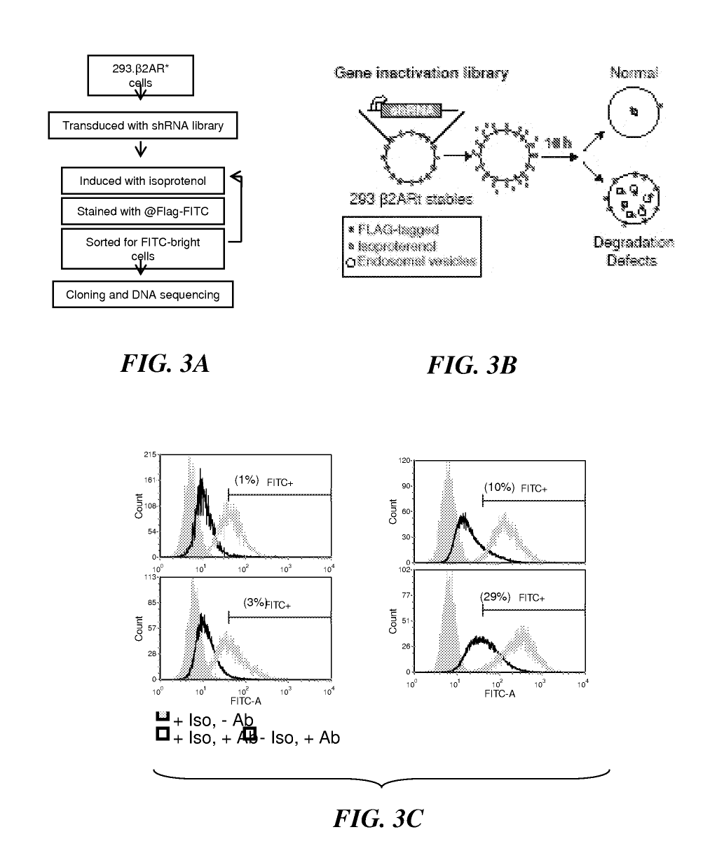

Fluorescence activated cell sorting (FACS)-based approach was used to repeatedly enrich cells with higher amount of surface β2AR phenotype from the library cells post isoproterenol induction (FIG. 3). To prepared cells for sorting, the cells were gently disassociated with low concentration trypsin and labeled them with FITC-conjugated anti-FLAG antibody. The inventors performed a total of 4 rounds of FACS sorting with about 10 millions cells at each sorting to make sure the full coverage of library (see Methods). The inventors also limited the culturing time for expanding cell numbers before each sorting. In the final (4th) sorting, cells with higher amount of cell surface receptor represented about 29% of the population. At the same sorting conditions, this represented only 1% in the initial unsorted library cells. Thus, the inventors achieved a 29-fold enrichment of a small cell population with higher amount of surface β2AR, which might be the result of down regulation of candidate genes by library shRNAs.

The inventors herein have established an HEK293 cell line (designated as 293 β2AR*) which stably expresses a modified β2AR that undergoes robust down-regulation upon agonist (isoproterenol: ISO) stimulation (FIG. 1A). These cells where then adapted for a flow cytometry assay to monitor the cell surface amount of the receptor. The 293β2AR cells were transduced with high-titer EST-derived shRNA lentiviral library to establish a genome-wide cellular shRNA library. The size of the cellular library was estimated to be more than 5 million (individual viral integration events), providing a good coverage of the genes in the library. The inventors used a FACS-based approach to isolate from the library cells with higher amount of undegraded β₂AR at the cell surface after ISO induction (FIG. 1B,C). These FACS-enriched cells (e.g. FACS positive cells) contain shRNA inactivation in genes that are critical for the agonist-induced β2AR down-regulation. To identify the shRNAs, the inventors extracted genomic DNAs from the FACS sorted cells and performed genomic PCR and identified sequences of about 100 shRNAs.

The inventors then extracted genomic DNAs from the FACS sorted cells and performed PCR to amplify the shRNA expressing sequences. We have obtained sequences of total about a hundred shRNA clones and identified their corresponding target genes by BLAST search on the human DNA sequence databases at National Center for Biotechnology Information (NCBI). About two third of the sequences matched perfectly (26- or 25-bp) to over a dozen of target genes. Interestingly, a gene targeted by a third of the shRNA clones is a GPCR gene, Latrophilin 2. Some other target genes have predicted function in vesicle trafficking, while some have known functions in lipid biosynthesis.

The 100 shRNAs sequences correspond to 15 human genes, as shown in Table 1. The inventors have validated and mechanistically characterized at least five of the identified gene hits. Those genes are VAMP-A like Protein, Tubulin Polymerization-Promoting Protein Family Member 3 (TPPP3), Arrestin Domain Containing 3 (ARRDC3), latrophilin 2 (LPHN2), Guanine Nucleotide Binding Protein-like 3 (nucleolar)-like Protein, Dolichyl-Phosphate Mannosyltransferase Polypeptide 2, Farnesyl Diphosphate Synthase (FDPS), and Low Density Lipoprotein Receptor-related Protein 5., NEDD4, CAMKK2, PELP1, SNAPC5, GGT7, BZW1L1, BZW1L1 (pseudogene 1), GGN, GNL3L, NPIP and FBXO44. The inventors transduced 10 different shRNAs clones individually back to 293β2AR* cells and measured the amount of cell surface β2AR.

The inventors discovered that at least 4 clones had moderate inhibitory effect of agonist induced β2AR internalization i.e. increase the amount of cell surface β2AR by about 20% as compared to empty vector (data not shown). One of the clones with the 26-nt hairpin sequence (5′-AGC GGA GAA AGT GAC CTA GAG ATT G-3′ (SEQ ID NO: 20)) targets the farnesyl diphosphate synthase (FDPS), which is a key enzyme in the mevalonate pathway (Roelofs, 2006; Russell, 2006).

Using a novel EST-derived shRNA expressing library, several regulators for β2AR down regulation were discovered. Some of them have predicted function in vesicle trafficking, while some have known functions in lipid biosynthesis.

The inventors further demonstrate that FDPS gene silencing and FDPS pharmacologic blockade (alendronate) decreases β2AR down-regulation and leads to elevated cell surface receptor levels (including in physiologically relevant, primary human ASM cells). The mechanism for FDPS action is likely to be through cholesterol biosynthesis, as cholesterol depletion also blocks β2AR down-regulation and, conversely, cholesterol supplementation reverses the effects of FDPS blockade.

Interestingly, one regulator, ARRDC3 (discussed in Example 4 herein) showed very high structure similarity to a family of proteins, Arrestins that regulate GPCR desensitization and internalization (Marchese, et al., 2008; Moore, et al., 2007). Herein, the inventors have demonstrated that ARRDC3 promote β2AR degradation by regulating its ubiquitination.

The inventors have also demonstrated that ARRDC3, which is required for receptor ubiquitination, is an essential step in receptor down-regulation. ARRDC3 co-localizes and interacts with β2AR in an agonist-dependent manner, and also interacts with Nedd4, the ubiquitin E3 ligase previously shown to ubiquitinate β2AR. These results demonstrate that ARRDC3 may function as an adaptor to recruit NEDD4 to ubiquitinate agonist-activated β2AR. Together, the inventors have identified new β2-AR regulator genes, and inhibition of these genes decreases β2-AR down-regulation, as well as established their roles at the cellular level.

Given the clinical importance of β2AR down-regulation, the inventors have demonstrated that these novel β2-AR regulator genes play critical role in mediating physiological receptor function in vivo.

### Example 3

### FDP Synthase Regulates the Amount of Cell Surface NAR

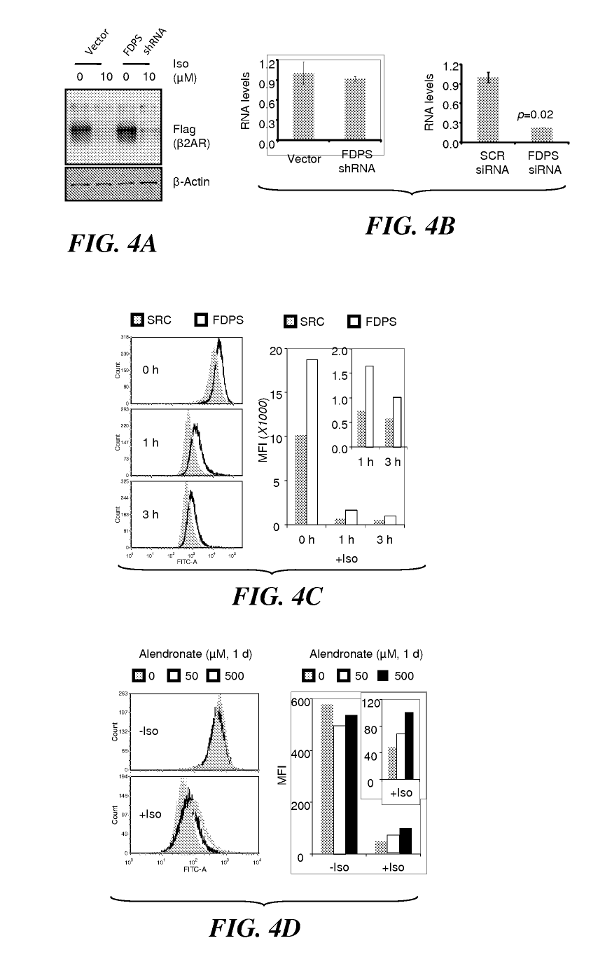

To confirm the inhibitory effect of FDPS-targeting shRNA on agonist induced β2AR internalization, the inventors re-transduced the shRNA back to 293β2AR* cells. Immunoblot analysis showed increased amount of β2AR in FDPS-specific shRNA transduced cells after 3 hours of agonist induction as compared to empty vector transduced cells (FIG. 4A). RNAi technology is known to have potential side effects caused by imperfect match to non-target sequences. To limit such side effect, the inventors used a synthetic siRNA for FDPS with different sequence to the shRNA we had isolated. The FDPS-specific siRNA down regulated the mRNA level of FDPS efficiently by about 80%, while the shRNA had only about 10% efficiency (FIG. 4B). Functionally, FDPS-specific siRNA transfected 293.β2AR* cells showed about 2-fold increase in cell surface β2AR both prior and post agonist induction as compared to non-targeting control siRNA transfected cells (FIG. 4C).

To further validate the role of FDPS, the inventors used a chemical inhibitor of FDPS, alendronate to treat 293.β2AR* cells for 1 day with two different doses. Such treatment induced dose dependent increase of the cell surface β2AR levels after agonist induction (FIG. 4D). Similar to FDPS siRNA, alendronate treatment for a longer time (e.g., 2 days) could increase the surface β2AR levels in 293.β2AR* cells both prior and post agonist induction (data not shown).

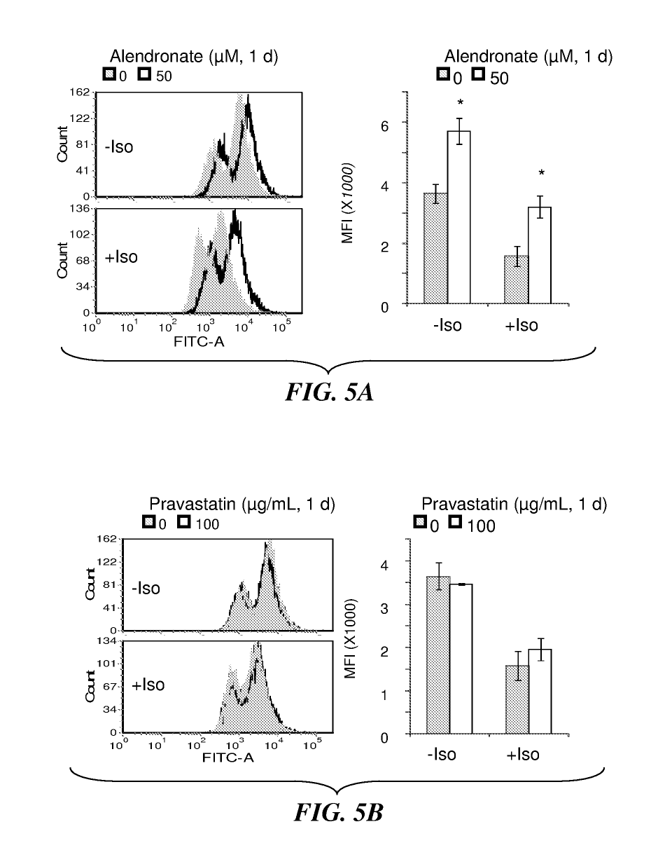

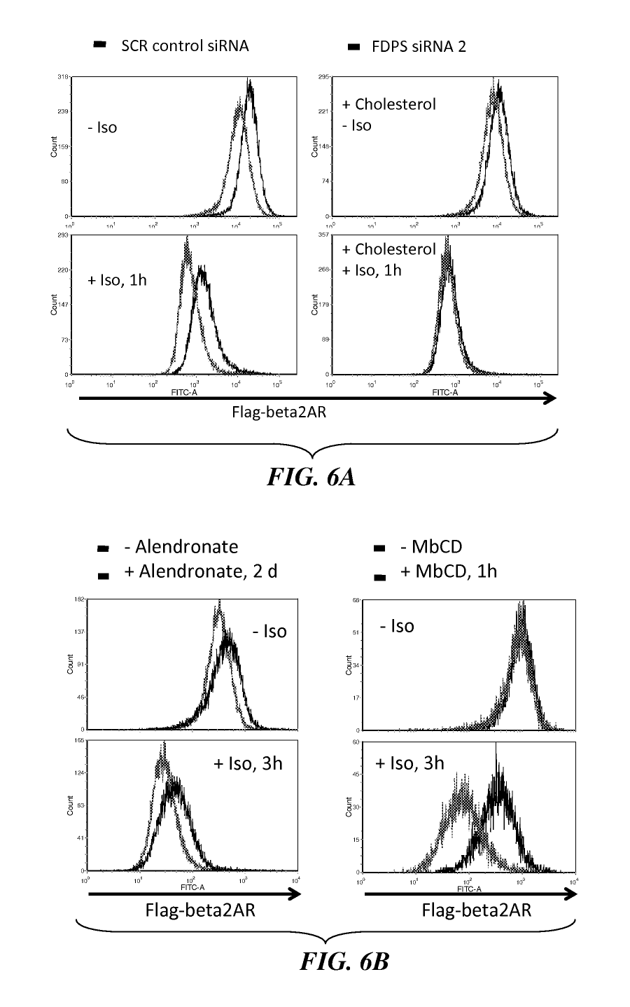

The inventors then tested if FDPS would have the same effects on wild type β2AR as was detected on the recycle-deficient β2AR. Thus the inventors used another cell line expressing the Flag-tagged wild type β2AR (293. β2AR^{WT}). The inventors treated the 293.β2AR^{WT} cells with the FDPS inhibitor alendronate for 1 day before the induction of β2-AR internalization using a β2-AR agonist. The inventors measured the amount of cell surface β2AR by flowcytometry. The inventors demonstrated that alendronate treatment increased the cell surface β2AR levels both before and after agonist induction in 293.β2AR^{WT} cells (FIG. 5A).

FDPS is a key enzyme in the melonvate pathway and the rate limiting enzyme in this pathway is first upstream enzyme, Hydroxymethylglutaryl-coenzyme A (HMG-CoA) reductase. Therefore, inhibition of the HMG-CoA reductase will attenuate the all the downstream activities including those mediated by FDPS. The inventors then tested HMG-CoA reductase to determine if the amount of cell surface β2AR was similar to inhibition of FDPS. The inventors demonstrated that treatment of the HMG-CoA reductase inhibitor, pravastatin for 1 day had no significant effect on regulating the amount of cell surface β2AR in 293 (FIG. 5B). β2AR^{WT} cells.

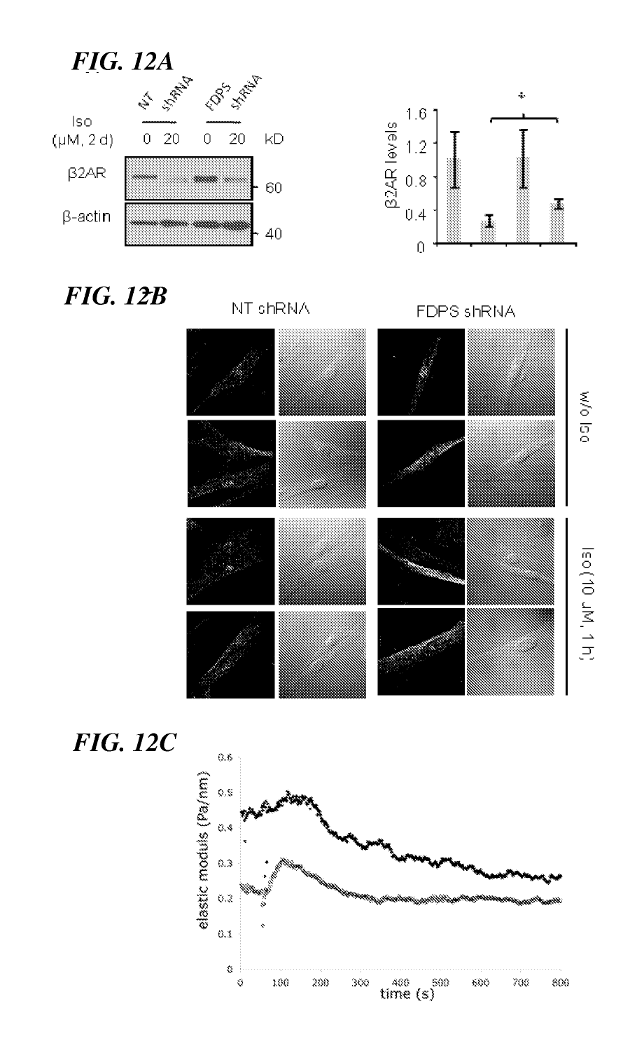

Finally, the effect of FDPS gene silencing was tested on β2AR in its physiological relevant (therapeutic target) cell type, the human airway smooth muscle (HASM) cells. Immunoblot analysis showed the expressions of β2AR were down regulated in HASM cells by agonist treatment of 2 days. FDPS knockdown increased the β2AR level in agonist treated HASM cells as compared to non-targeting control (FIG. 12A). Immunofluorescence staining revealed that there were no obvious changes in subcellular localization of β2AR in FDPS knockdown cells (FIG. 12B), therefore demonstrating that the enhancement of β2AR level occurred with both cell surface and intracellular β2AR. Functionally, FDPS gene silencing also affect the elasticity of HASM cells. β-agonist treatment could reduce the elasticity of HSAM cells thus made cells less stiff. The mechanical property of FDPS knockdown was measured in HSAM cells using optical magnetic twisting cytometry (OMTC) (FIG. 12C). The mechanical assays demonstrated that the control cells are twice as stiff as the FDPS knockdown cells. Additionally, upon stimulation with isoproterenol, the elastic modulus of both cell types decreased within a few hundreds of second and reached a plateau. The stiffness of the control cells decreased by ˜40% and the sample cells decreased by ˜15% compared to their respective baselines.

In summary, demonstrated herein that FDPS is a β2-AR regulator gene and can regulate the amount of cell surface β2AR. Inhibition of FDPS by gene silencing or nitrogen-containing diphosphate could increase the amount of both recycle-deficient and wild type β2AR on the cell surface. Functionally, FDPS gene silencing also affected β2AR expression and cell elasticity in HASM cells.

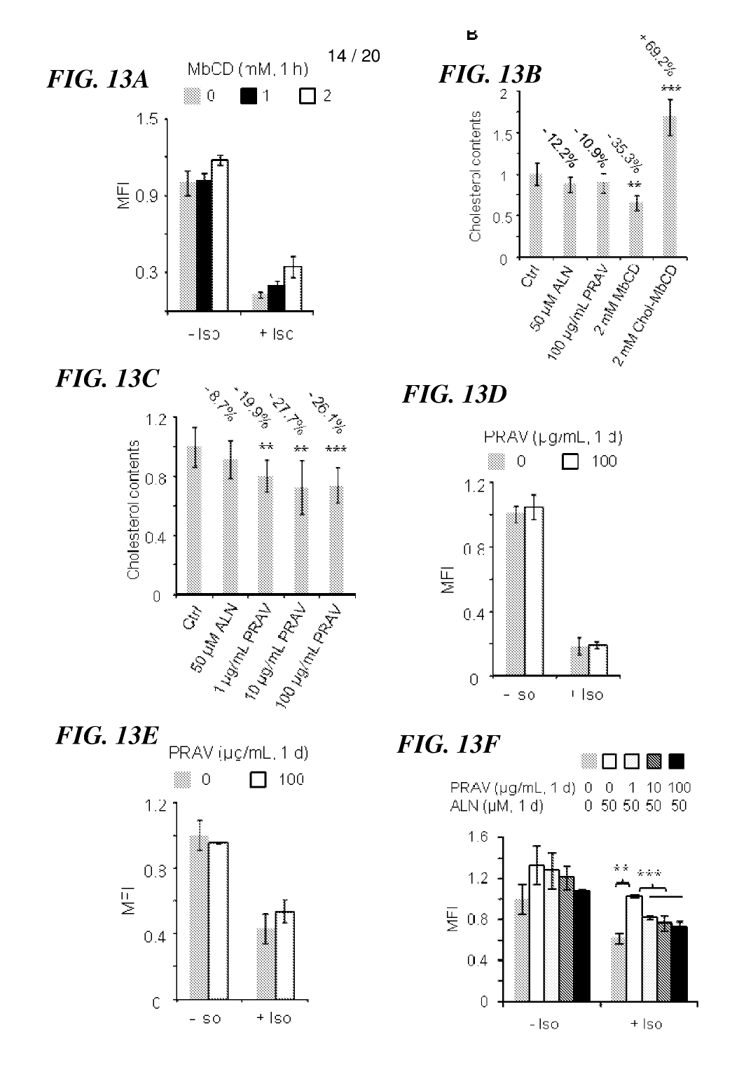

FDPS Regulates PAR through Cholesterol-independent Mechanisms. FDPS is a key enzyme in the mevalonate pathway and the rate limiting enzyme in this pathway is first upstream enzyme, HMGCR. Therefore, inhibition of the HMGCR will attenuate the all the downstream activities including those mediated by FDPS. The efficiency of inhibition of FDPS and HMGR should correlate to the downstream biosynthetic activities, one of which is cholesterol synthesis. Cholesterol plays critical role in maintaining cell membrane structures. It has been previously reported that β2AR resided in cholesterol rich lipid rafts and cellular cholesterol contents could regulate β2AR signaling (Insel, 2005; Pontier, 2008; Xiang, 2002). To assess a potential role of cholesterol in regulating β2AR down regulation, cells were treated with Methyl-beta-cyclodextrin (MbCD), a reagent for acute cholesterol depletion. It was demonstrated that 293.β2AR* cells pretreated with MbCD for 1 hour did show increased amount of cell surface β2AR post agonist induction (FIG. 13A).

Next, the cellular cholesterol contents was measured of FDPS or HMGR-inhibited 293.β2AR* cells using alendronate or pravastatin treatments respectively. Two controls were used; MbCD and cholesterol saturated MbCD (Cholesterol-MbCD). The effects of cholesterol lowering by MbCD (lower 35.3%) and cholesterol increasing by Cholesterol-MbCD (increase 69.2%, FIG. 13B) was detected. However, neither alendronate nor pravastatin could significantly lower the cellular cholesterol (FIG. 13B). To limit such compensation effect, the cholesterol measurement was performed using serum-free culture medium, which determined that pravastatin treatment efficiently lower the cellular cholesterol even at very low concentration (1 μg/mL, lower 19.9%, FIG. 13C). Controversially, alendronate treatment was still not sufficient to lower cellular cholesterol in serum-free medium. Inhibition of FDPS by alendronate may turn on the cholesterol feed-back mechanisms that up regulate HMGCR to counteract the inhibitor effect.

The effect of pravastatin inhibition on HMGR was assessed to determine if it affected the amount of cell surface β2AR similar or was different to FDPS inhibition. 1 day treatment of the HMGR inhibitor, pravastatin had no significant effect on increasing the amount of cell surface β2AR in both 293.β2AR* (FIG. 13D) and 293.β2AR^{WT} cells (FIG. 13E). Therefore, it was determined that FDPS inhibition regulated β2AR through alternation of upstream whether than downstream activities of the mevalonate pathway.

FDPS inhibition resulted in accumulation of an upstream intermediate, which could also have important functional consequences in different systems (Roelofs, et al., 2009; Raikkonen, et al., 2009; Monkkonen, et al., 2008; Monkkonen, et al., 2007; Monkkonen, et al., 2006; Thompson, et al., 2004; Gober, et al., 2003). Inhibition of the further upstream enzyme HMGCR could diminish such accumulation thus reverse the effects of FDPS inhibition (Thompson, et al., 2004). To determine whether such mechanism played a role in our study, 293.β2AR^{WT} cells were treated with both alendronate and pravastatin. Surprisingly, pravastatin reversed the effect of alendronate on β2AR regulation. Treatment with alendronate alone increased the amount of cell surface β2AR (FIG. 13F). Adding pravastatin diminished the effect of alendronate in a dose dependent manor. Therefore, it is demonstrated that FDPS inhibition regulated the β2AR down regulation by accumulation of intermediate product(s) that is downstream of HMGCR but upstream of FDPS, thus further indicated the cholesterol independent mechanisms.

### Example 4

Receptor down regulation contributes greatly to β-agonist tolerance and limits the efficacy of regular or long term β-agonist treatment for asthma and COPD. Such adverse effect can also lead to poorer disease control and asthma-related death. Naturally, there is a human β2AR allele contains a single nucleotide polymorphism associated with accelerated down regulation (Liggett, et al., 2000; Moore, et al., 2000). Individuals who are homozygous of such β2AR allele also showed reduced response to β-agonists (Tan, et al., 1997) and more severe adverse outcomes in long-term β2-agonist treatment (Taylor, et al., 2000; Israel, et al., 2004). Therefore, interventions slowing the β2AR down regulation could be beneficial for those individuals in special and might prevent the development of tolerance in long term use of (3-agonists in general.

Studies in β2AR receptor down regulation have greatly advanced our knowledge on the molecular mechanisms governing such important cellular process. Until recently, characterization of gene functions needs individually laborious investigation. The inventors have used a genome-wide shRNA screen to readily identify novel regulators for β2AR function. The inventors have developed a novel EST-derived shRNA library having several advantages. The lentiviral library can transduce a wild range of cell types. It derives from a large EST collection that contains normalized copies for each gene. It expresses long (26-bp) shRNAs that are much more efficient than the widely used 19 to 21-bp shRNAs. Also, the library has higher numbers (˜20) of shRNA sequences for each average human gene. Such higher numbers could increase the chance of getting shRNA sequences that would produce efficient knockdown. However, as it also increases the number of non-efficient sequences, it recommended that one perform genetic screen using multiple copies of the library to prevent random loss of efficient shRNAs.

FDPS, a key upstream and “branching” enzyme in the mevalonate pathway. Herein, the inventors have validated the effects of FPDS inhibition by both gene silencing and chemical inhibition. FPDS inhibition increased the cell surface amount of recycle-deficient β2AR and wild type β2AR in cell lines; evaluated β2AR expression and decreased cell elasticity in HASM cells. Such effects seemed to be independent of β-agonist induction. Moreover, such effects did not correlate to cholesterol synthesis, an important downstream activities of mevalonate pathway.

It was discovered herein that acute depletion of cellular cholesterol could also inhibit β2AR down regulation (FIG. 13A). Nevertheless, cellular cholesterol content depends on the balance of de novo synthesis, uptake, reverse transport, and utilization. Also, cholesterol synthesis is auto-regulated by the rate of flux through the mevalonate pathway, and the feed-back control point is HMGCR. Therefore, inhibition of neither FDPS nor HMGCR did not lower cellular cholesterol efficiently in serum-containing medium because of uptake compensation (FIG. 13B) (Cole, 2005). With limited uptake compensation in serum-free medium, HMGCR inhibition did efficiently lower cellular cholesterol while FDPS inhibition still could not (FIG. 13C), demonstrating existence of a feed-back up regulation of HMGCR (FIG. 13C). Indeed, the inventors have demonstrated that FDPS inhibition might perturbs the upstream activities of the mevalonate pathway. According to studies in other systems, FDPS inhibition resulted in accumulation of an upstream intermediate, isopentenyl diphosphate (Roelofs, et al., 2009; Raikkonen, et al., 2009). HMGCR inhibition could diminish such accumulation (Thompson, et al., 2004), accounting for the reverse effect of pravastatin on alendronate (FIG. 13F).

Nitrogen-containing bisphosphonates (e.g. alendronate) are potent inhibitors of FDPS, and now used as drugs for bone diseases associated with excessive resorption. These drugs inhibit the function of osteoclasts by disrupting the isoprenoid lipid modification of small GTPases. More interestingly, there are resent evidence indicate that accumulation the substrate of FPDS, isopentenyl diphosphate and one of its metabolite also contribute to the nitrogen-containing bisphosphonates induced apoptosis of osteoclasts (Raikkonen, et al., 2009). Furthermore, these drugs are new candidates for cancer chemotherapy because they can potently inhibit cell proliferation. Persistent asthma or COPD causes airway remodeling/narrowing and proliferation of airway smooth muscle cells is responsible for such consequence. β-agonist treatment in asthma or COPD may have other expulmonary adverse effect because the wild tissue distribution of β-adrenergic receptors. Besides of the most common cardiovascular adverse effect, osteoporosis might be another potential adverse effect. Osteoporosis is known to associate with COPD although it is hard to separate disease effect from treatment effect. Clearly, use of β-agonists can cause osteoporosis through signaling in oesteoblasts in laboratory animals. Since nitrogen-containing bisphosphonates are efficient drugs for osteoporosis, it will be also interesting to know if they could limit such co-morbidity in COPD. Herein, the inventors have demonstrated that FDPS as a novel regulator for β2AR and demonstrate a method for using nitrogen-containing bisphosphonates and β-agonists for the treatment of COPD and other disorders.

Both β2AR signaling and mevalonate pathway are important targets for many pharmaceutical drugs. The present invention provides for combined therapeutics to increase efficacy, for example by increasing the amount of cell surface receptor. Furthermore, the combined therapeutics comprising an inhibitor of FDPS and a β2-AR agonist can be used to reduce certain complications such as proliferation of airway smooth muscle cells. Accordingly, the inventors have discovered that compounds which inhibit FDPS are useful in such combined therapeutics, for example with β2-AR agonists.

### Example 5

Prolonged stimulation of the β2-adrenergic receptor (β2AR) leads to receptor ubiquitination and downregulation. Using a genome-wide RNA interference screen discussed in Example 1, the inventors identified ARRDC3 as a gene required for β2AR regulation. ARRDC3 protein interacts with ubiquitin ligase NEDD4 through two conserved PPXY motifs and recruits NEDD4 to activated receptor. ARRDC3 also interacts and colocalizes with activated β2AR. Knockdown of ARRDC3 abolishes the association between NEDD4 and β2AR. Functional inactivation of ARRDC3 through either siRNA knockdown or overexpression of a mutant that does not interact with NEDD4 blocks receptor ubiquitination and degradation. The inventors have discovered that ARRDC3 as an essential adaptor for β2AR ubiquitination, and that inhibition of ARRDC3 inhibits β2-AR ubiquitination and subsequent degredation.

### Identification of ARRDC3 as a Novel Gene Required for β2AR Downregulation.

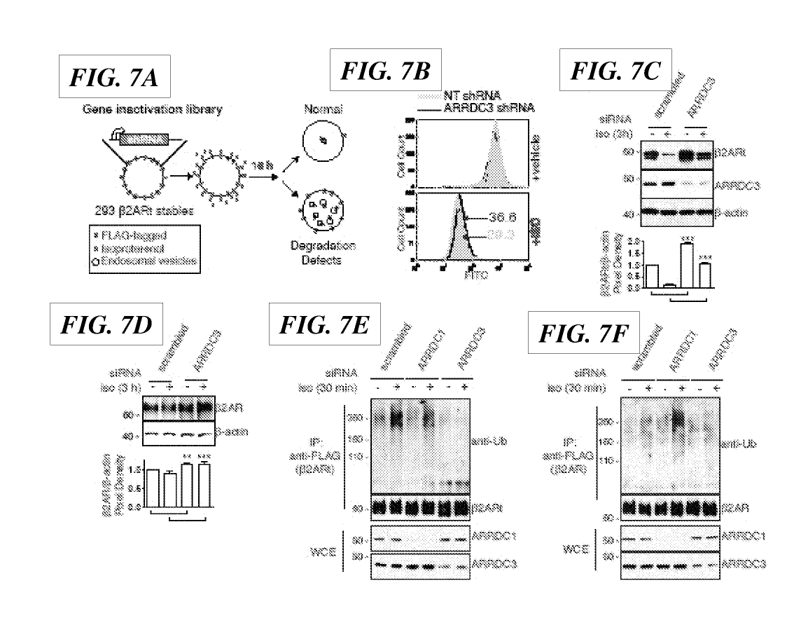

The inventors performed a RNA interference (RNAi)-based genetic screen to identify genes required for β2AR downregulation (FIGS. 3A, 3B and 7A). The screen was carried out in a cell line that expresses a distally truncated form (after residue 385) of β2AR (β2ARt), which undergoes fast and efficient receptor degradation upon agonist stimulation (Cao et al, 1999; Gage et al, 2001). Briefly, β2ARt cells were transduced with a lentiviral genome-wide shRNA (short hairpin RNA) library and subjected to agonist (isoproterenol:ISO) stimulation for 16 h. The inventors then performed multiple rounds of fluorescence-activated cell sorting (FACS) to enrich for cells that, after ISO stimulation, maintained high levels of β2AR, indicative of possible defects in β2AR degradation. shRNAs that potentially interfere with β2AR downregulation were identified from the sorted cell population by genomic PCR and sequencing.

Two of the identified shRNAs corresponded to a novel gene ARRDC3 (arrestin domain-containing 3), also known as TLIMP (Oka et al, 2006). To confirm the effect of ARRDC3 shRNA-mediated knockdown on β2AR downregulation, the inventors transduced β2ARt cells with lentiviral particles carrying the identified ARRDC3 shRNA sequence. Flow cytometry analysis showed that cells expressing the ARRDC3-specific shRNA exhibited higher (>20%) level of cell surface receptor after agonist stimulation, compared with the control non-targeting shRNA expressing cells (FIG. 7B).

To ascertain that the stabilization of β2AR was due to ARRDC3 knockdown and was not caused by a potential off-target effect of the shRNA, the inventors tested the effect of an ARDDC3 siRNA targeting a sequence different from that of the identified shRNA. ARRDC3 protein expression was efficiently knocked down (>75%) by the siRNA and, as a result, ISO-induced degradation of β2ARt was dramatically inhibited: the level of un-degraded receptor in ARRDC3-knockdown cells was about 6-fold higher than that in the scrambled control siRNA-transfected cells (FIG. 1C). This result demonstrates that ARRDC3 is required for agonist-stimulated β2AR degradation. The ARRDC3-knockdown cells also exhibited a slight increase (<2-fold) in the basal level (prior to ISO stimulation) of the receptor, suggesting that ARRDC3 may also play a role in the slow yet constant steady-state receptor degradation in the absence of an agonist (Morrison et al, 1996). To confirm that the effect of ARRDC3 knockdown on β2AR degradation is not limited to the modified β2ARt, the inventors determined the effect of siRNA-knockdown on the degradation of the wild-type β2AR (FIG. 7D). Consistent with previous studies (Cao et al, 1999), wild-type β2AR exhibited slow degradation (−10%) upon ISO stimulation. Nevertheless, such slow degradation of β2AR was blocked in ARRDC3 siRNA-transfected cells (FIG. 7D). Together, the inventors have demonstrated that ARRDC3 as a β2-AR regulator gene that is required for the efficient degradation of β2AR.

ARRDC3 belongs to a protein family that includes five other arrestin-domain containing proteins (Alvarez, 2008; Aubry et al, 2009). Most of the ARRDC proteins have no annotated biological functions. However, two of the ARRDCs (ARRDC1 and ARRDC3), when expressed as GFP-fusion proteins, localize to the plasma membrane (data not shown), suggesting membrane-related functions. To determine whether ARRDC1, like ARRDC3, plays a role in ISO-induced β2AR degradation, the inventors examined the effect of ARRDC1 knockdown. Despite efficient knockdown (>90%) of ARRDC1 using siRNA, β2AR degradation was not affected (data not shown).

### ARRDC3 is Required for Agonist-Dependent Ubiquitination of β2AR

Since ubiquitination is a prerequisite for efficient agonist-induced receptor degradation (Shenoy et al, 2001), the inventors tested whether ARRDC3 plays a role in β2AR ubiquitination. The inventors determined the level of β2AR ubiquitination by immunoprecipitation (IP) of β2AR followed by anti-ubiquitin immunoblot analysis. As shown in FIG. 7E, ISO stimulation led to a significant increase in the amount of ubiquitinated species of β2AR in both scrambled control siRNA- and ARRDC1 siRNA-transfected cells, but the increase in ubiquitination was strongly inhibited in ARRDC3-knockdown cells (−70% reduction in normalized amount of ubiquitinated β2AR compared to ISO-treated control). Such effect was specific for β2AR ubiquitination as ARRDC3 depletion did not grossly alter total ubiquitination level (data not shown). Similarly, knockdown or inhibition of ARRDC3 also diminished agonist-dependent ubiquitination of wild-type β2AR (−30% reduction in ubiquitinated β2AR compared to ISO-treated control; FIG. 7F). Consistent with the degradation data (data not shown), knockdown of ARRDC1 did not inhibit β2AR ubiquitination (FIG. 7E and FIG. 7F). Thus, the inventors have demonstrated that ARRDC3 is required for agonist-dependent ubiquitination of β2AR, and that inhibition or gene silencing of ARRDC3 results in inhibiting β2-AR agonist significantly reduced (up to 70%) β2-AR ubiquitination and subsequent degradation.

ARRDC3 Interacts with and Recruits NEDD4 E3 Ligase to Mediate β2AR Ubiquitination

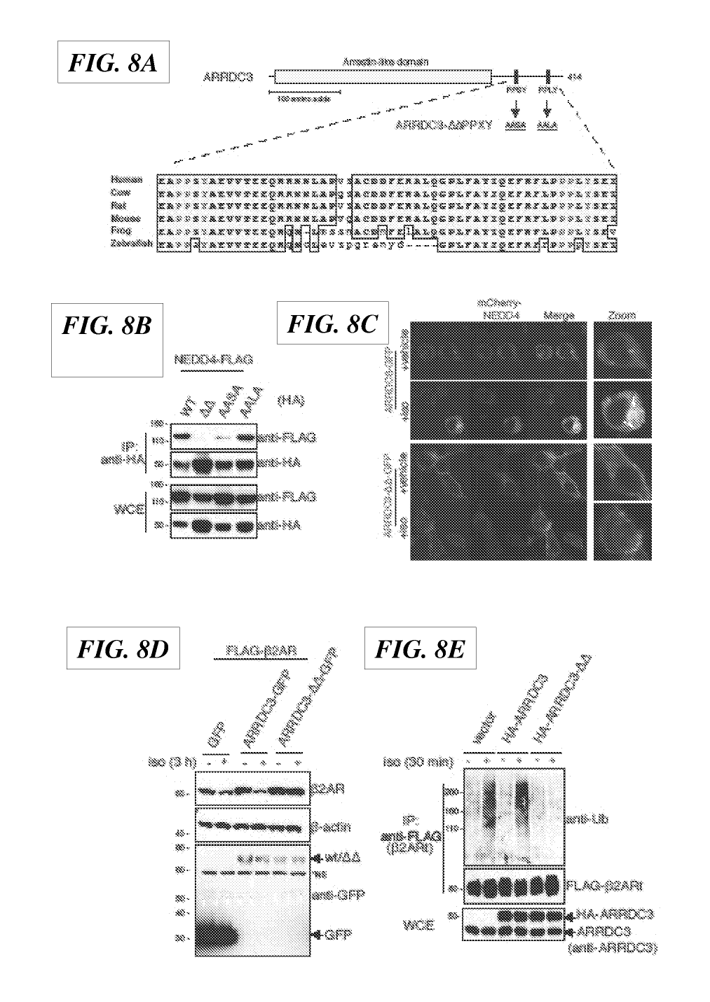

The human ARRDC3 protein contains at its C-terminus two PPXY motifs that are conserved among other orthologues (FIG. 8A). Since PPXY motifs are known to mediate protein-protein interactions through interaction with the WW domains (Rotin & Kumar, 2009), the inventors investigated if ARRDC3 interacts with the WW domain-containing NEDD4, which was characterized recently as a ubiquitin ligase for β2AR (Shenoy et al, 2008), to facilitate β2AR ubiquitination. Indeed, the inventors demonstrate that ARRDC3 interacts with NEDD4 in a co-IP experiment (FIG. 8B). To determine whether the PPXY motifs in ARRDC3 are required for the observed interaction with NEDD4, the inventors generated PPXY mutants (for either single or double motifs) and showed that, while the first PPXY motif plays a more important role in the interaction, both PPXY motifs had to be mutated to fully abrogate the interaction of ARRDC3 with NEDD4 (FIG. 2B). Together, the inventors have demonstrated that ARRDC3 interacts with NEDD4 through the conserved PPXY motifs.

To further explore the ARRDC3/NEDD4 interaction, the inventors examined whether ARRDC3 and NEDD4 colocalize with each other. The inventors expressed two fluorescent fusion proteins (ARRDC3-GFP and mCherry-NEDD4) in 293β2AR cells. ARRDC3-GFP, when expressed alone, is localized mostly the plasma membrane (data not shown). However, co-expression with mCherry-NEDD4 led to a cytosolic localization of ARRDC3 (FIG. 8C, upper panel). This is likely due to steady-state association between ARRDC3 and NEDD4, resulting in the partial redistribution of ARRDC3 to the cytosol where NEDD4 resides. Upon addition of agonist, both mCherry-NEDD4 and ARRDC3-GFP were found at the vicinity of the plasma membrane and in discrete bodies in the cytosol where they colocalized (FIG. 8C). In contrast, ARRDC3ΔΔPPXY-GFP showed little colocalization with mcherry-NEDD4 either prior or upon agonist-stimulation (FIG. 2C lower panels). These data demonstrate and reinforce the notion that ARRDC3 interacts with NEDD4 through the PPXY motifs.

To determine whether the ARRDC3/NEDD4 interaction is required for the degradation of β2AR, the inventors next tested the effect of overexpression of ARRDC3ΔΔPPXY, which does not interact with NEDD4. As shown in FIG. 8D, expression of the double PPXY ARRDC3 mutant inhibited ISO-dependent β2AR degradation, while the control GFP or wild-type ARRDC3-GFP did not. Moreover, expression of ARRDC3ΔΔβPPXY blocked β2AR ubiquitination, whereas vector or ARRDC3-transfected controls displayed robust receptor ubiquitination following ISO stimulation (FIG. 8E). Thus the inventors have clearly demonstrated that ARRDC3/NEDD4 interaction is required for β2 AR ubiquitination and subsequent degradation.

ARRDC3 Interacts with β2AR in an Agonist-Dependent Manner

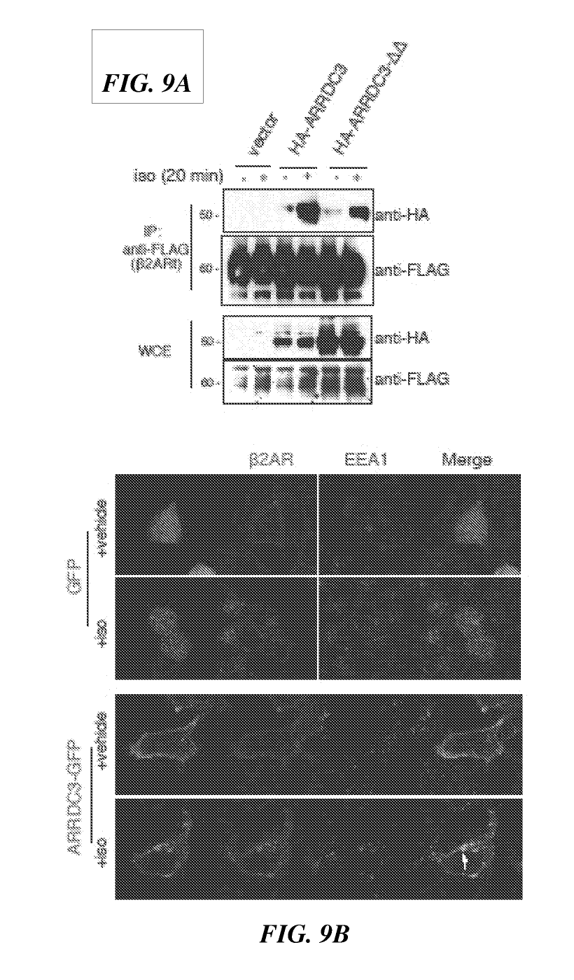

The N-terminal region of ARRDC3 is homologous to a conserved (arrestin) domain in the arrestin proteins, which interact with activated (phosphorylated) GPCRs (Gurevich & Gurevich, 2006) and play an important role in attenuating GPCR signaling (DeWire et al, 2007). The inventors assessed if ARRDC3 is recruited to the β2-AR upon binding of β2-AR agonist. The inventors determined whether ARRDC3 associates with β2AR. As shown in the co-IP experiments (FIG. 9A), ARRDC3 interacted with β2AR. Although a weak association was observed prior to ISO addition, the ARRDC3/β2AR interaction was greatly enhanced after β2-AR agonist stimulation. This interaction does not require the PPXY motifs in ARRDC3 as the double PPXY mutant was also associated, though less robustly, with activated β2AR (FIG. 9A).

The inventors further determined that the ARRDC3/β2AR interaction occurred by examining the colocalization of the two proteins. Cells expressing FLAG-β2AR were transfected with control GFP or ARRDC3-GFP, and receptors were visualized by immunostaining with anti-FLAG antibodies. Upon ISO stimulation, β2AR was internalized from the plasma membrane into cytosolic early endosomal vesicle dots as indicated by co-immunostaining with the early endosomal marker EEA1 (FIG. 9B). This was evident in both GFP and ARRDC3-GFP transfected cells. Prior to ISO stimulation, ARRDC3-GFP almost exclusively localized to the plasma membrane where β2AR also resides. Upon agonist stimulation, some of the ARRDC3 signal internalized into cytosolic vesicles (data not shown) that colocalize with both β2AR and EEA1 (FIG. 9B right panels). This is consistent with the ARRDC3/β2AR interaction observed in the co-IP experiment, and suggests that ARRDC3 may continue to associate with β2AR after agonist stimulation and during receptor endocytosis. Similarly, colocalization of ARRDC3 with the fast-degrading receptor was observed (data not shown). Colocalizations with the receptors were also observed in the PPXY mutant (data not shown) albeit only at the membrane. Together, the inventors have demonstrated that ARRDC3 associates with β2AR and that such an association is enhanced upon β2-AR agonist stimulation, and that ARRDC3 inhibition leads to decreased β2-AR internalization and degredation.

### ARRDC3 Mediates the Association Between NEDD4 and NAR

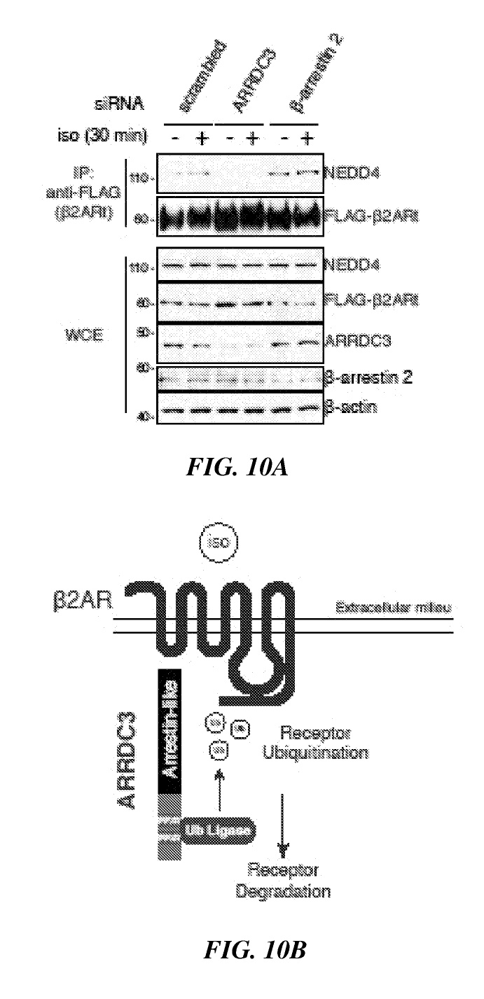

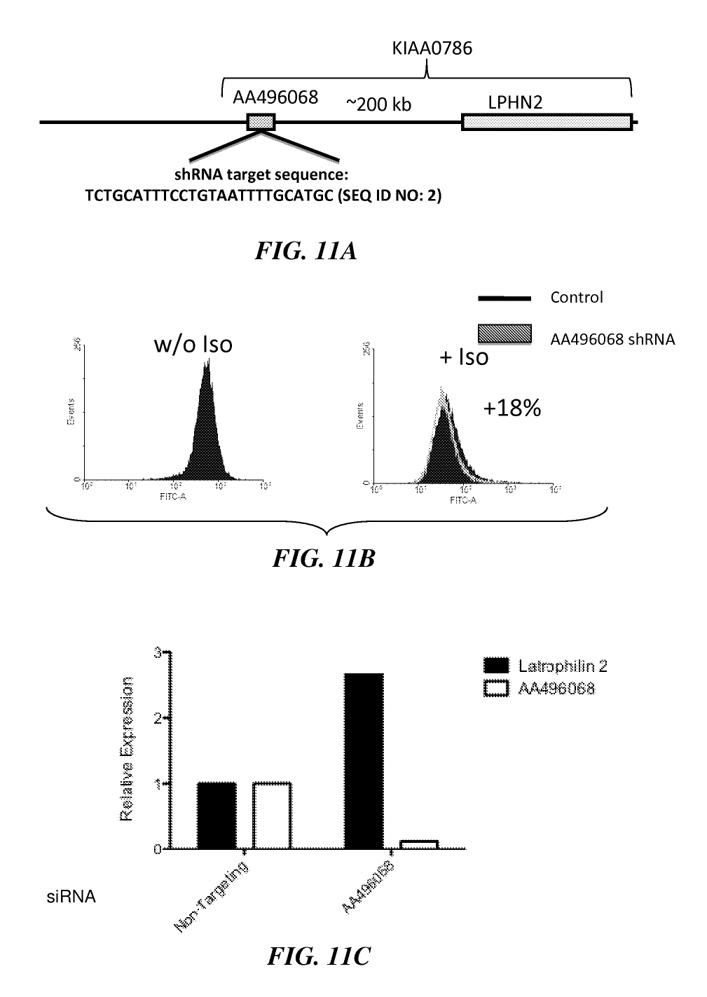

NEDD4 was previously found to associate with β2AR upon agonist stimulation and was previously reported that β-arrestin-2 mediates the NEDD4/β2AR association (Shenoy et al, 2008). The inventors next determined whether ARRDC3 is required for the association between NEDD4 and activated β2AR. The inventors transfected β2ARt-expressing cells with siRNAs targeting ARRDC3, β-arrestin-2 or a non-targeting sequence, and then assessed the NEDD4/β2AR interaction using an IP assay. As shown in FIG. 10A, NEDD4 associated with β2AR in scrambled control siRNA-transfected cells, and such association was moderately augmented upon ISO stimulation. Knockdown of ARRDC3 by siRNA completely abrogated the association of NEDD4 with the receptor in the presence or absence of ISO stimulation. In contrast, a significant reduction of β-arrestin-2 protein levels (˜60% reduction compared to control) did not affect the interaction between NEDD4 and β2AR (FIG. 4A). Thus, the inventors have demonstrated that ARRDC3 is an adaptor that mediates the association between NEDD4 and β2AR.

In summary of Example 4, the inventors have discovered that ARRDC3 as an essential component of the β2AR downregulation process. The inventors show in FIG. 10B a schematic illustration of a model to illustrate the function of ARRDC3 (FIG. 10B). ARRDC3 interacts with NEDD4 through its PPXY motifs and associates with β2AR at the cell surface before agonist stimulation but much more robustly thereafter. Through the interaction with ARRDC3, NEDD4 is recruited to the proximity of the receptor, allowing the E3 ligase to recognize and ubiquitinate the receptor.

Although the inventors have clearly demonstrated that both ARRDC3 and β-arrestin-2 are required for the efficient □2AR downregulation, the inventors have also demonstrated that ARRDC3 is the adaptor that recruits NEDD4 to the activated β2-AR Unlike ARRDC3, β-arrestin-2 plays an important role in receptor internalization by recruiting components of the clathrin-mediated internalization machinery (such as AP2) (Santini et al, 2002). The two proteins may coordinate β2AR downregulation: ARRDC3 through direct interaction with the ubiquitin ligase, and β-arrestin-2 by recruiting essential components of the internalization machinery.

The ubiquitination adaptor role of ARRDC3 is remarkably similar to that of ART (Arrestin-Related Trafficking adaptor) proteins in yeast. ARTs have been shown to recruit the yeast HECT domain E3 ligase, Rsp5, to ubiquitinate target plasma membrane proteins (Lin et al, 2008). Although the sequence similarity is low between ARRDC3 and the yeast ART proteins (data not shown; (Alvarez, 2008)), they have a similar functional domain organization: an arrestin-like domain at the N-terminus and two PPXY motifs at the C-terminus (Alvarez, 2008; Lin et al, 2008). The conservation of structural domains and the analogous functions of the ARTs and ARRDC3 indicate that the ubiquitin ligase adaptor functions have been conserved throughout evolution. Similar to yeast ARTs, there are multiple ARRDC proteins in humans (data not shown). The fact that the other plasma membrane-localized ARRDC, ARRDC1, has no obvious role in β2AR ubiquitination indicates little functional redundancy among the ARRDC family members Unlike yeast, which has only one HECT-domain ligase (Rsp5), mammalian cells have at least 9 such ubiquitin ligases (Rotin & Kumar, 2009), all of which (including NEDD4) contain WW domains that may interact with PPXY motifs. Indeed, ARRDC3 interacts with at least three other HECT domain E3 ligases (data not shown). It is thus possible that ARRDC3 through its interactions with other members of the NEDD4 E3 ligase family may function as an ubiquitination adaptor for multiple receptor protein substrates.

The inventors have demonstrated herein that ARRDC3 is an important mediator of β2-AR ubiquitination and stability. Meticulous control of receptor levels at the cell surface through downregulation ensures appropriate levels of receptor-mediated signaling in tissues such as the lung airways. On the other hand, excessive receptor downregulation can lead to reduction or loss of functional membrane-associated β2AR, thus severely limiting therapies (i.e., β2-AR agonists for asthma treatment) that rely on efficient β2AR activation and signaling (Shore & Moore, 2003). Use of agents which inhibit ARRDC3 with β2-AR agonists may prolong β2-AR agonist function, and are thus useful in the treatment of disorders of the heart and lung, and respiratory disorders which typically use β2AR-based therapies.

### Example 6

Most of the other β2-AR regulator genes as disclosed in Table 1 have not been previously linked to β2AR regulation. Using gene-specific siRNAs targeting sequences that are different from the identified shRNAs (to minimize potential “off-target” effects), the inventors have validated the role of three other top hits (CaMKK2 and KIAA0786) in β2AR regulation.

CaMKK2 variants have been previously associated with acute bronchodilator response (BDR). The inventors examined whether genetic variants in the newly identified β2AR regulator genes correlate with clinical bronchodilator response (BDR). GWAS on the CAMP parent-child trios 45 was previously completed by Dr. Tantisira and collegues. There were a total of 226 SNPs in 9 of the 15 RNAi gene hits on the Illumina HumanHap 550vs Beadchip used in the GWAS. The inventors tested the association of these SNPs with BDR, which was treated as a continuous trait using an additive model 46 and adjusted for age, sex, height and baseline FEV 1. Seven SNPs in four genes were nominally associated with acute BDR at randomization (Table 2).

TABLE 2
BDR-associatedSNP

| Gene | SNP (rs#_allele) | P value |
|---|---|---|
| CaMKK2 | rs1653593_2 | 0.00997 |
| CaMKK2 | rs1140886_2 | 0.04608 |
| CaMKK2 | rs11609701_4 | 0.08065 |
| KIAA0786 | rs987042_1 | 0.05578 |
| KIAA0786 | rs11163408_2 | 0.09844 |
| PELP1 | rs17823987_2 | 0.02404 |
| SNAPC5 | rs12594835_2 | 0.06022 |

It was discovered that CaMKK2 has a high association with BDR. Three of the 8 CaMKK2 SNPs analyzed were associated with BDR, which was more than one would be expected by chance. All of the three SNPs are intronic, and they were also associated with BDR measured at 11 times over 4 years (p values similar to those for BDR at randomization; data not shown). Interestingly, all of the minor alleles for the three CaMKK2 SNPs (rs1140886 (G), rs11609701 (T), and rs1653593 (C)) are associated with an increase in BDR. While the association results were modest, this emphasizes the value of the inventors approach of crossing the results of the GWAS study with the results of our RNAi screen, to identify clinically relevant, functional genes that might not have otherwise been detected in GWAS. Moreover, since the Illumina Chip used in the GWAS is based on maximizing linkage disequilibrium coverage 45, the identified BDR-associated CaMKK2 SNPs may not be the actual functional variants, which may have a stronger clinical association. Aim 2 seeks to identify the functional CaMKK2 variants.

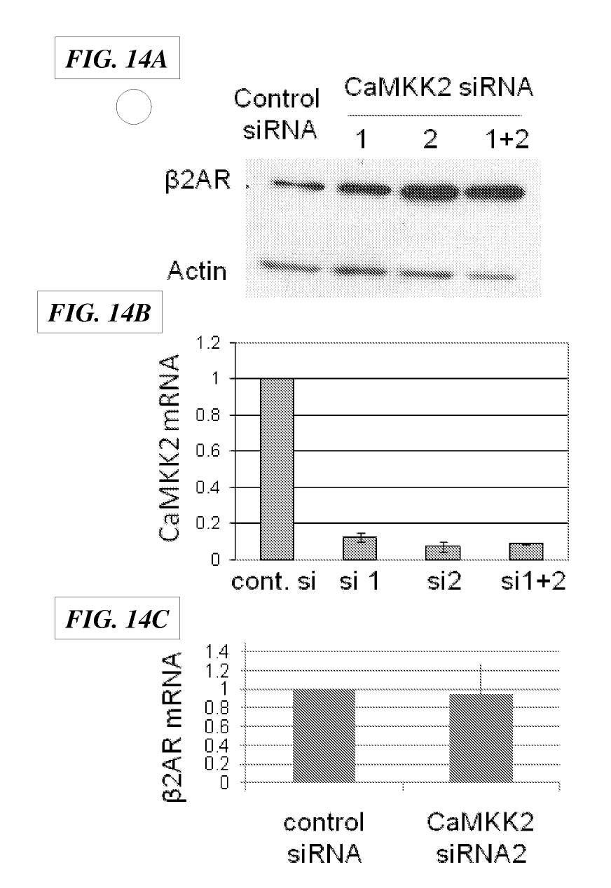

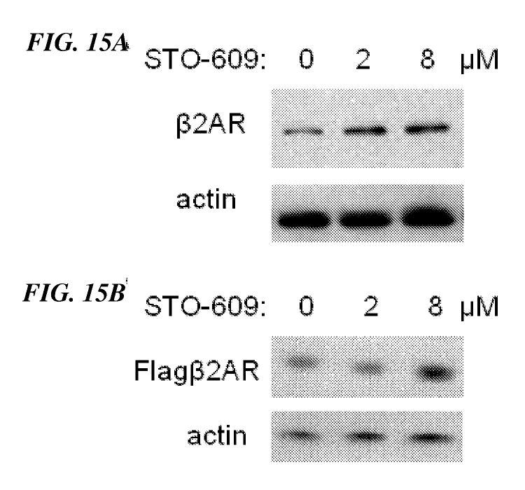

The inventors then demonstrated that siRNA-mediated knockdown of CaMKK2 increases β2AR protein level in primary airway smooth muscle cells. The inventors demonstrated the effect of CaMKK2 inhibition on β2AR in the primary human airway smooth muscle cells (HASM), which express β2AR and are the physiological target of β-agonists. Treatment of human airway smooth muscle (HASM) cells with two CaMKK2-specific siRNAs 5′ GCUCCUAUGGUGUCGUCAAdTdT (CaMKK2 siRNA #1) (SEQ ID NO: 32) and 5′ UUGACGACACCAUAGGAGCdTdT (CaMKK2 siRNA #2) (SEQ ID NO: 33), both of which efficiently knocked down CaMKK2 expression by about 90% (FIG. 14B), led to more than 2-fold increase in β2AR protein level (FIG. 14A). The effect correlated with the level of CaMKK2 knockdown, as a better knockdown by siRNA #2 resulted in a bigger increase (>3 fold) in β2AR protein. In contrast to the effect on β2AR protein, CaMKK2 siRNA did not affect the β2AR mRNA level as measured by qRT-PCR (FIG. 14C).

Chemical inhibition of CaMKK2 increases β2AR protein level. To further establish the role of CaMKK2 on β2AR, the effect of STO-609, a potent inhibitor of CaMKK2 was investigated. Treatment of STO-609 at as low as 2 μM resulted in an increase in β2AR in the HASM cells (FIG. 4A). STO-609 treatment at a higher concentration also increased exogenously expressed β2AR protein in the 293β2AR cells (FIG. 15B).

### Example 7

In addition to the gene modulators identified in Table 1, the inventors demonstrated a micro RNA transcript which functions as an inhibitor of β2-AR expression, and modulation by inhibition of such micro RNA transcript increases β2-AR expression.

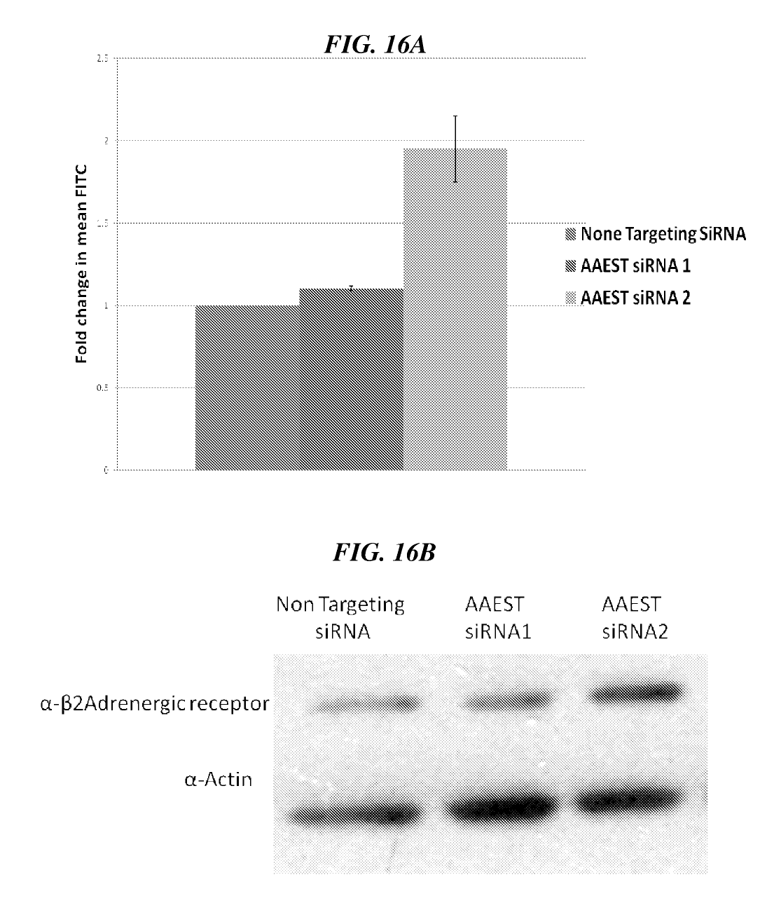

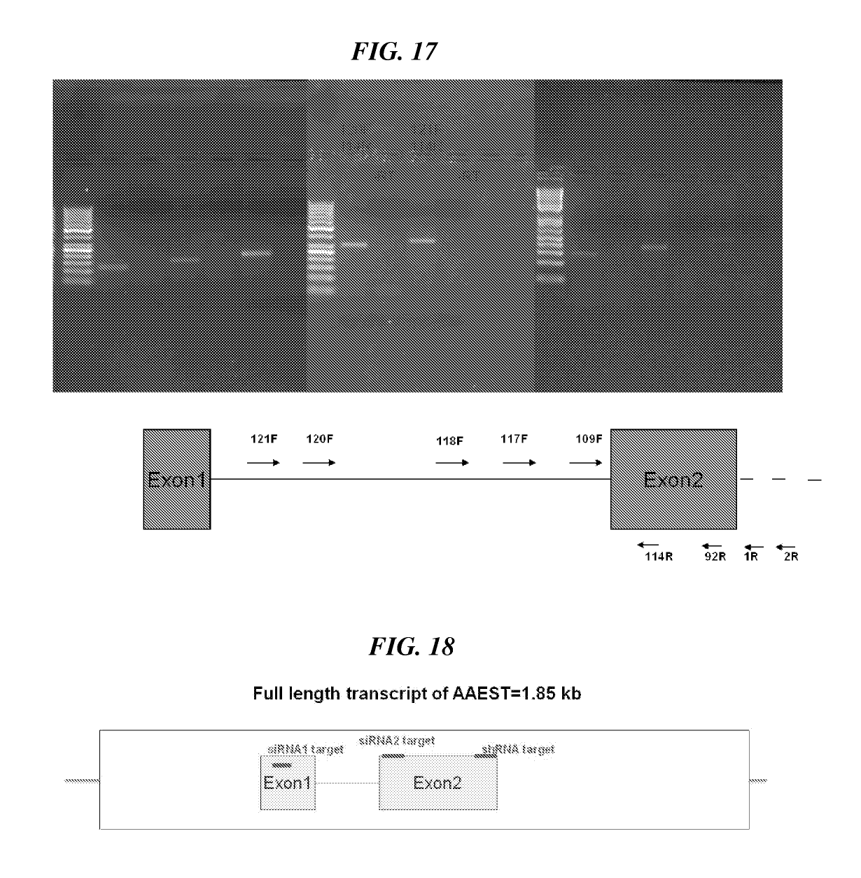

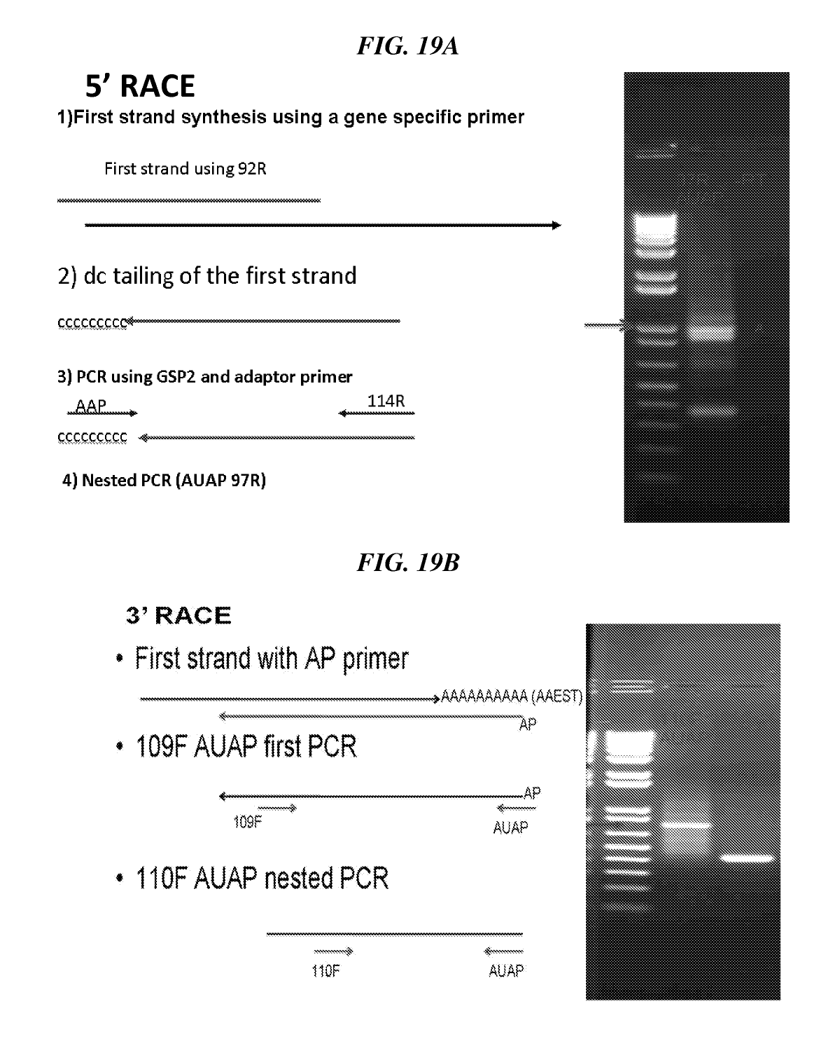

In particular, the inventors demonstrate in FIG. 16A that inhibition of AAEST by siRNA knockdown, e.g., using siRNAs 5′ UAAUAUUUAAUGAGGCGGCCUdTdT (SEQ ID NO: 34) and/or 5′ AGGCCGCCUCAUUAAAUAUUAdTdT (SEQ ID NO: 35) decreases the presence of β2-AR on the surface of Human Airway Smooth Muscle (HASM) cells and also decreased total β2-AR protein expression (FIG. 16B). On cloning the full length AAEST transcript using primers (FIG. 17), the inventors were able to clone the entire full length sequence using 5′ and 3-RACE (FIG. 19A-19B) and discovered the full length gene is 1.85 kb. The full length sequence of AAEST is as follows (the numbers indicate the position of the nucleotide sequences on human Chromosome 1):

| (SEQ ID NO: 36) | |
|---|---|
| gatgaata actccacagc tcctcctgga | 81970583 |
| ccctgcgcgg gagcaggcgg ctcctgtgct gtaaagaaaa ttgattcgct | 81970633 |
| tgcggctcac ctcagtcgag gaagccctgg aatgttcagc agaacaccac | 81970683 |
| cactgtgaca tggggctgtg gcagtgggag acaccgcatg tggcgtgggt | 81970733 |
| gtgtgtggcg gcgtggctcg cactcagtcc tgtgtaggag aggaaaggga | 81970783 |
| ATCAAAAGCT TGAGCACAAA CAGATACCTC AGCCCGgtaa gctgcagctc | 81970833 |
| tgctcaactg cgtcttctct cagccctcca cacacgctca cccccactcc | 81970883 |
| cacacacaca cacacacaca cacacacaca ccatctcccg agggctgacc | 81970933 |
| tcctctggcc tcagcctgca gggtgtgggc agagaagggc atctgggacg | 81970983 |
| tggtgccagt gaggagccca gttggctggc actgggccca tttgaaggtg | 81971033 |
| tctcagacat ttggccagta tgtctttctc aggggtttgg tcacaaagga | 81971083 |
| tggactcttc ccacccagag gatgcaggga aagcacactg tgtctttccg | 81971133 |
| gtcattggat cctctccctt tccccaggca gctcgcctgg ccacaccgtt | 81971183 |
| ggagtgaacc ctcactgccc tcaaggacaa cagcagggtg tcacccagag | 81971233 |
| ccggatgagg gatgccagca ggtgccccca cgagtggggc cttggcccaa | 81971283 |
| gcagagcttc ccctgaaggt gccatcagcc agggcagctc tgtcccctcc | 81971333 |
| tgctctccat cttatatgta ttctaaccag gaaaaatgtg atagcacatg | 81971383 |
| ggtagcctag gcagtgaata aatacctcag atgtcctcct gcaaaaaaaa | 81971433 |
| aaaaaaaaaa aaaaatagga ggctgaaacc tagaactgag aaaaatctga | 81971483 |
| gtttttatta aaaaaaagca cgtttttact ttctgatatc cacctcagct | 81971533 |
| tttgttcttt aaaatgggat caatgtcatt acacaatttt cattaaaatc | 81971583 |
| atgtaaaaag caccacgctg tgcaaaagat gggcccaaat actctgcaaa | 81971633 |
| gatcattgca cgtaaatcag atcctttccc tctacctgta gGAGTTTCTG | 81971683 |
| TTCCTGTTCT TGAAGAGACA GACTGGTGAG CATGCAAAAT TACAGGAAAT | 81971733 |
| GCAGAGAACA AAATGGGCAG AGCAACCAAA ACTGTGGTGT TTGCATCAAA | 81971783 |
| TACAGGTCAA GAGTAAACTT ATTTTCCTAT GAAATTCAAG AACATGTTGA | 81971833 |
| AACTGGAAAG AGCGGGACAG GCTGGTAGCA CCTTTTAAAG ACCAAGAGAG | 81971883 |
| GCCGCCTCAT TAAATATTAA GAACTTGGAG GAAAGAGGTG GATTTACACT | 81971933 |
| GATAAAAGGT TCATTTAAAA TTCCATGAGG TCAATAAATT ACCACTTAag | 81971983 |
| atgccatttc ccaaaatgtg tcctgaagaa tgcttgtttt aaatgagggt | 81972033 |
| GGAGGGGTAG AGGGAAAAAA TCCTGTGGTC CAATTGATTT ATGCACTGTA | 81972083 |
| TCTCAGATGG AGTTTGACAA GAAATGTTGG CCAGGCGCGG TGTATCAAAC | 81972133 |
| CTGTAATCCC AGCACTTTGG GAGGCTGAGG TGGGAAGGAT CACTTGAGAC | 81972183 |
| CAGGAGTTTG AGACCAGCCT GGATGACATA AGGAGACCCC ATCTCTACAA | 81972233 |
| ATAATTAAAA AATTAGCCAG GTGTGTCAGT GCACACCTGT GGTCTCAGCT | 81972283 |
| ACTCAGGAGG CTGAGGCAAG AGGATCACCT AAGGCCAGGA GGTTGAGGCT | 81972333 |
| GCAGTGAGAT GTGATGGCAC CACTGCACTC CAGCCTGGGT GACTGAGTGA | 81972383 |
| GACCCAGTCT CAAAAGAACA AAACAAAACA AAACAAAACA AAACAAAACA | 81972433 |
| AAACAAAAAA CCAGACAATA AATTGTTGAG TTACCAAAGG CCCTGTTAAC | 81972483 |
| GCCTGCAACA AAAAAGATTG CTGAATCTTG CTTAGTCCAG TCTTTTCCAA | 81972533 |
| ACTTGTTTGA CCTTCAACCC CTTTCCTCTT CCTCTGCACA TTCATCTACA | 81972583 |
| TAACAAATGT ACAGAGGGCC TCCTCCAGGC CAGTCACTGT TCTAGGTCCT | 81972633 |
| ggggattcag cagtgaagaa aacaaaactc tcccttggca gactgtgtaa | 81972683 |
| ttaggaggag acagaaaata aaccaataga taaatcaaac cagaa | |

Determining the Mode of Regulation of AAEST on β2-Adrenergic Receptor

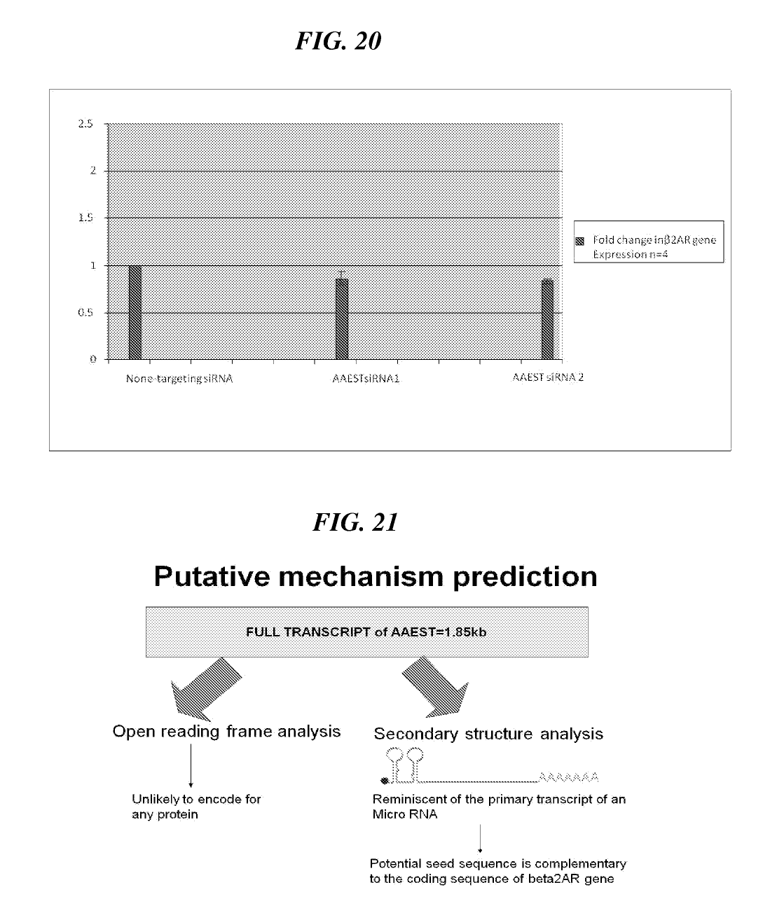

The inventors also performed quantitative RT-PCR on Human Airway Smooth Muscle (HASM) cells in the presence and absence of AAEST siRNA. As shown in FIG. 20, AAEST siRNA did not decrease the mRNA expression level of β2-adrenergic receptor (2β2-AR), demonstrating that as AAEST can decrease β2-AR protein expression (see FIG. 16A-16B) but not 213-AR mRNA expression, AAEST functions to inhibit β2-AR gene expression at the protein level.

The inventors also performed additional sequence analysis (see FIG. 21) and determined that AAEST functions as a primary transcript of a novel micro RNA (referred to herein as a miRNA or mirRNA), which acts as an inhibitor of β2AR expression through translational repression.

Accordingly, herein, the inventors have discovered that AAEST is a novel transcript that can form miRNA. AAEST is a small piece of RNA transcript that is expressed from the putative first intron of KIAA0786. The inventors have demonstrated that inhibition by siRNA knocking down of the AAEST transcript leads to an increase in baseline β2AR expression in cells. Accordingly, AAEST transcript can be combined with GWAS studies to link observed association between bronchodilator phenotype and SNPs in this region

Additionally, inhibition of AAEST, e.g., using siRNAs as disclosed herein can be used for treatment of asthma and COPD to improve β-agonists based asthma therapy by increasing β-AR baseline expression and prevent β-agonist-induced downregulation.

### REFERENCES

All references cited in the specification and the Examples are incorporated herein in their entirety by reference.
Alvarez C E (2008) On the origins of arrestin and rhodopsin. BMC Evol Biol 8: 222
Aubry L, Guetta D, Klein G (2009) The arrestin fold: variations on a theme. Curr Genomics 10(2): 133-142
Cao T T, Deacon H W, Reczek D, Bretscher A, von Zastrow M (1999) A kinase-regulated PDZ-domain interaction controls endocytic sorting of the beta2-adrenergic receptor. Nature 401(6750): 286-290
DeWire S M, Ahn S, Lefkowitz R J, Shenoy S K (2007) Beta-arrestins and cell signaling. Annu Rev Physiol 69: 483-510
Gage R M, Kim K A, Cao T T, von Zastrow M (2001) A transplantable sorting signal that is sufficient to mediate rapid recycling of G protein-coupled receptors. J Biol Chem 276(48): 44712-44720
Gurevich V V, Gurevich E V (2006) The structural basis of arrestin-mediated regulation of G-protein-coupled receptors. Pharmacol Ther 110(3): 465-502
Hanyaloglu A C, von Zastrow M (2008) Regulation of GPCRs by endocytic membrane trafficking and its potential implications. Annu Rev Pharmacol Toxicol 48: 537-568
Hershko A, Ciechanover A (1998) The ubiquitin system. Annu Rev Biochem 67: 425-479
Lin C H, Macgurn J A, Chu T, Stefan C J, Emr S D (2008) Arrestin-related ubiquitin-ligase adaptors regulate endocytosis and protein turnover at the cell surface. Cell 135(4): 714-725
Marchese A, Paing M, Temple B, Trejo J (2008) G Protein-Coupled Receptor Sorting to Endosomes and Lysosomes. Annu Rev Pharmacol Toxicol 48: 601-629
Mittal R, Mcmahon H T (2009) Arrestins as adaptors for ubiquitination in endocytosis and sorting. EMBO Rep 10(1): 41-43
Morrison K J, Moore R H, Carsrud N D, Trial J, Millman E E, Tuvim M, Clark R B, Barber R, Dickey B F, Knoll B J (1996) Repetitive endocytosis and recycling of the beta 2-adrenergic receptor during agonist-induced steady state redistribution. Mol Pharmacol 50(3): 692-699
Oka S-i, Masutani H, Liu W, Horita H, Wang D, Kizaka-Kondoh S, Yodoi J (2006) Thioredoxin-binding protein-2-like inducible membrane protein is a novel vitamin D3 and peroxisome proliferator-activated receptor (PPAR)gamma ligand target protein that regulates PPARgamma signaling. Endocrinology 147(2): 733-743
Penn R B, Benovic J L (2008) Regulation of heterotrimeric G protein signaling in airway smooth muscle. Proc Am Thorac Soc 5(1): 47-57
Pierce K L, Premont R T, Lefkowitz R J (2002) Seven-transmembrane receptors. Nat Rev Mol Cell Biol 3(9): 639-650
Premont R T, Gainetdinov R R (2007) Physiological roles of G protein-coupled receptor kinases and arrestins. Annu Rev Physiol 69: 511-534
Rotin D, Kumar S (2009) Physiological functions of the HECT family of ubiquitin ligases. Nat Rev Mol Cell Biol 10(6): 398-409
Santini F, Gaidarov I, Keen J H (2002) G protein-coupled receptor/arrestin3 modulation of the endocytic machinery. J Cell Biol 156(4): 665-676
Shenoy S K, McDonald P H, Kohout T A, Lefkowitz R J (2001) Regulation of receptor fate by ubiquitination of activated beta 2-adrenergic receptor and beta-arrestin. Science 294(5545): 1307-1313
Shenoy S K, Xiao K, Venkataramanan V, Snyder P M, Freedman N J, Weissman A M (2008) Nedd4 mediates agonist-dependent ubiquitination, lysosomal targeting, and degradation of the beta2-adrenergic receptor. J Biol Chem 283(32): 22166-22176
Shore S A, Moore P E (2003) Regulation of beta-adrenergic responses in airway smooth muscle. Respiratory physiology & neurobiology 137(2-3): 179-195
1. Bateman, E. D. et al. Global strategy for asthma management and prevention: GINA executive summary. Eur Respir J 31, 143-78 (2008).
2. Tattersfield, A. E., Knox, A. J., Britton, J. R. & Hall, L P. Asthma. Lancet 360, 1313-22 (2002).
3. Donohue, J. F. et al. Long-term safety of nebulized formoterol: results of a twelve-month open-label clinical trial. Ther Adv Respir Dis 2, 199-208 (2008).
4. Mansfield, L. E. The future of the long-acting beta-adrenergic bronchodilators in the treatment of asthma. Allergy Asthma Proc 29, 103-8 (2008).
5. McGraw, D. W. & Liggett, S. B. Molecular mechanisms of beta2-adrenergic receptor function and regulation. Proc Am Thorac Soc 2, 292-6; discussion 311-2 (2005).
6. Shore, S. A. & Moore, P. E. Regulation of beta-adrenergic responses in airway smooth muscle. Respir Physiol Neurobiol 137, 179-95 (2003).
7. Johnson, M. The beta-adrenoceptor. Am JRespir Crit. Care Med 158, 5146-53 (1998).
8. Lipworth, B. J. Airway subsensitivity with long-acting beta 2-agonists. Is there cause for concern? Drug Saf 16, 295-308 (1997).
9. Nelson, H. S. & Dorinsky, P. M. Safety of long-acting beta-agonists. Ann Intern Med 145, 706; author reply 708-10 (2006).
10. Nelson, H. S., Weiss, S. T., Bleecker, E. R., Yancey, S. W. & Dorinsky, P. M. The Salmeterol Multicenter Asthma Research Trial: a comparison of usual pharmacotherapy for asthma or usual pharmacotherapy plus salmeterol. Chest 129, 15-26 (2006).
11. Shore, S. A. & Drazen, J. M. Beta-agonists and asthma: too much of a good thing? J Clin Invest 112, 495-7 (2003).
12. Liggett, S. B. Polymorphisms of beta-adrenergic receptors in heart failure. Am J Med 117, 525-7 (2004).
13. Liggett, S. B. Genetic variability of the beta2 adrenergic receptor and asthma exacerbations. Thorax 61, 925-7 (2006).
14. Tattersfield, A. E. & Hall, I. P. Are beta2-adrenoceptor polymorphisms important in asthma—an unravelling story. Lancet 364, 1464-6 (2004).
15. Hancox, R. J., Sears, M. R. & Taylor, D. R. Polymorphism of the beta2-adrenoceptor and the response to long-term beta2-agonist therapy in asthma. Eur Respir J 11, 589-93 (1998).
16. Israel, E. et al. The effect of polymorphisms of the beta(2)-adrenergic receptor on the response to regular use of albuterol in asthma. Am JRespir Crit. Care Med 162, 75-80 (2000).
17. Taylor, D. R. et al. Asthma exacerbations during long term beta agonist use: influence of beta(2) adrenoceptor polymorphism. Thorax 55, 762-7 (2000).
18. Lu, Q., Wei, W., Kowalski, P. E., Chang, A. C. & Cohen, S. N. EST-based genome-wide gene inactivation identifies ARAP3 as a host protein affecting cellular susceptibility to anthrax toxin. Proc Natl Acad Sci USA 101, 17246-51 (2004).
19. The Childhood Asthma Management Program (CAMP): design, rationale, and methods. Childhood Asthma Management Program Research Group. Control Clin Trials 20, 91-120 (1999).
20. Lange, C. et al. A family-based association test for repeatedly measured quantitative traits adjusting for unknown environmental and/or polygenic effects. Stat Appl Genet Mol Biol 3, Article 17 (2004).
21. de Bakker, P. I. et al. Efficiency and power in genetic association studies. Nat Genet. 37, 1217-23 (2005).
22. Barrett, J. C., Fry, B., Maller, J. & Daly, M. J. Haploview: analysis and visualization of LD and haplotype maps. Bioinformatics 21, 263-5 (2005).
23. Kohm, A. P., Mozaffarian, A. & Sanders, V. M. B cell receptor- and beta 2-adrenergic receptor-induced regulation of B7-2 (CD86) expression in B cells. J Immunol 168, 6314-22 (2002).
24. Silverman, E. S. et al. Transforming growth factor-beta1 promoter polymorphism C-509T is associated with asthma. Am J Respir Crit Care Med 169, 214-9 (2004).
25. Peters, S. P. et al. Randomized comparison of strategies for reducing treatment in mild persistent asthma. N Engl J Med 356, 2027-39 (2007).
26. Clinical trial of low-dose theophylline and montelukast in patients with poorly controlled asthma. Am J Respir Crit Care Med 175, 235-42 (2007).

1. (canceled)

2. A method for increasing β2 adrenergic receptor agonist function in a subject, comprising administering simultaneously, sequentially or separately, a composition comprising a β2 adrenergic receptor agonist and a composition comprising an inhibitor of farnesyl diphosphae synthase (FDPS).

3. (canceled)

4. (canceled)

5. (canceled)

6. (canceled)

7. (canceled)

8. The method of claim 2, wherein the inhibitor is a nucleic acid inhibitor.

9. (canceled)

10. The method of claim 8, wherein the nucleic acid inhibitor is a gene silencing RNAi agent.

11. (canceled)

12. (canceled)

13. (canceled)

14. The method of claim 2, wherein the inhibitor is a bisphosphonate or bisphosphate compound.

15. The method of claim 14, wherein the bisphosphonate or biosphosphate compound is alendronate or a salt thereof.

16. The method of claim 2, wherein the inhibitor is selected from the group consisting of: methyl-beta-cyclodextrin (MbCD), NE58025, risedronate, eetidronate, zoledronate, clodronate, ibandronate, incadronate, medronate, neridronate, oxidronate, pamidronate or tiludronate, ACETONEL™ or isedronate.

17. (canceled)

18. (canceled)

19. (canceled)

20. The method of claim 2, wherein the subject is a human.

21. The method of claim 2, wherein the subject is identified to have a respiratory disease prior to administration of the composition comprising a β2 adrenergic receptor agonist and a composition comprising an inhibitor of farnesyl diphosphae synthase (FDPS).

22. (canceled)

23. The method of claim 2, further comprising a first step of selecting a subject in need of β2-AR agonist therapy prior to the simultaneous, sequential or separate administration of a composition comprising a β2 adrenergic receptor agonist and a composition comprising an inhibitor of farnesyl diphosphae synthase (FDPS).

24. A composition comprising, in admixture, a β2 adrenergic receptor agonist and an inhibitor of farnesyl diphosphate synthase (FDPS).

25. (canceled)

26. (canceled)

27. (canceled)

28. (canceled)

29. (canceled)

30. (canceled)

31. The composition of claim 24, wherein the inhibitor is a nucleic acid inhibitor.

32. (canceled)

33. The composition of claim 31, wherein the nucleic acid inhibitor is a gene silencing RNAi agent.

34. The composition of claim 33, wherein the RNAi inhibitor agent is selected from any of: SEQ ID NO: 20, SEQ ID NO: 21, SEQ ID NO: 22 and SEQ ID NO: 23.

35. (canceled)

36. (canceled)

37. (canceled)

38. The composition of claim 24, wherein the modulator is a bisphosphonate or bisphosphate compound.

39. The composition of claim 38, wherein the bisphosphonate or biosphosphate compound is alendronate or a salt thereof.

40. The composition of claim 24, wherein the inhibitor is selected from the group consisting of: methyl-beta-cyclodextrin (MbCD)), NE58025, risedronate, eetidronate, zoledronate, clodronate, ibandronate, incadronate, medronate, neridronate, oxidronate, pamidronate or tiludronate, ACETONEL™ or isedronate.

41. (canceled)

42. (canceled)

43. (canceled)

44. The composition of claim 24, wherein the composition is an oral solution or in a form suitable for administration by inhalation.

45. (canceled)

46. A kit comprising a preparation of a beta-2 adrenergic receptor agonist, an inhibitor of farnesyl diphosphate synthase (FDPS) and optionally, instructions for the simultaneous, sequential or separate administration of the preparation to a subject in need thereof.

47. (canceled)

48. The method of claim 10, wherein the RNAi agent is selected from any of SEQ ID NO: 20, SEQ ID NO: 21, SEQ ID NO: 22 and SEQ ID NO: 23.

49. The method of claim 15, wherein the salt of alendronate is alendronate sodium triphosphate or alendronate monosodium salt trihydrate.

50. The composition of claim 39, wherein the salt of alendronate is alendronate sodium triphosphate or alendronate monosodium salt trihydrate.

51. The method of claim 2, wherein the subject has respiratory disease.

52. The method of claim 51, where the respiratory disease is asthma or chronic obstructive pulmonary disease (COPD).
